(19) United States
(12) Patent Application Publication
(10) Pub. No.: US 2003/0219427 A1
(43) Pub. Date: Nov. 27, 2003

(54) TPL-2/COT kinase and methods of use

(76) Inventors: Hamish J. Allen, Boylston, MA (US); Richard W. Dixon, Jefferson, MA (US); Joanne S. Kamens, Newton Centre, MA (US); Dineli Wickramasinghe, San Francisco, CA (US); Yajun Xu, Westborough, MA (US); Nancy Bump, Lowell, MA (US); Andres Salmeron, Westminster, MA (US); Monica P. Belich, London (GB); Leland H. Johnston, London (GB); Steven C. Ley, Hertfordshire (GB); Steven J. Gamblin, Hertfordshire (GB); Stephen J. Smerdon, London (GB); Soeren Beinke, Wienhausen (DE); Juergen Deka, Lausanne (CH)

Correspondence Address: ABBOTT BIORESEARCH, 100 RESEARCH DRIVE, WORCESTER, MA, 01605-4314, US

(21) Appl. No.: 10/374,414

(22) Filed: Feb. 26, 2003

Related U.S. Application Data
(63) Continuation-in-part of application No. 09/374,579, filed on Aug. 13, 1999, now abandoned.
(63) Continuation-in-part of application No. PCT/US99/18543, filed on Aug. 13, 1999.

(30) Foreign Application Priority Data
Dec. 16, 1998 (GB) GB9827712.2
Aug. 18, 1998 (GB) GB9817930.2

(51) Int. Cl. A61K 38/52

(52) U.S. Cl. 424/94.5

(57) ABSTRACT

It is shown that TPL-2 is responsible for phosphorylation of p105 and its resultant proteolysis, which leads to p50 Rel translocation to the nucleus. Accordingly, the invention provides TPL-2 as a specific regulator of the activation of NFκB, and thus as a modulator of inflammatory responses in which p1O5 is involved, and as a target for the development of compounds capable of influencing NFκB activation.

### RELATED INFORMATION

This is a continuation-in-part application of Ser. No.: 09/374579 and International Application No. PCT/US99/18543 filed on Aug. 13,1999, both of which are continuation-in-part applications of Ser. No.: GB9827712.2 filed on Dec. 16, 1998 which claims the benefit of priority to provisional application Ser. No.: GB9817930.2, filed on Aug. 18, 1998. The contents of the aforementioned applications and all other patents, patent applications, and references cited throughout this specification are hereby incorporated by reference in their entireties.

### BACKGROUND OF THE INVENTION

Nuclear Factor κ B (NFκB) was first discovered in 1986 as a nuclear factor involved in kappa light chain transcription in B cells (Sen and Baltimore, (1986) Cell 46:705-716) and has since been shown to be a ubiquitous transcription factor, existing in virtually all eukaryotic cell types (reviewed in Ghosh et al., (1998) Ann. Rev. Immunol. 16:225-260). In cells, NFκB exists in a cytoplasmic, inactive form complexed to an inhibitor protein, IκB. Upon stimulation with an appropriate inducer, IκB dissociates from NFκB and unmasks its nuclear localization signal, allowing transport into the nucleus, where its biological activity as a transcription factor is exerted. Thus, NFκB is a rapid modulator of gene expression, since its induction is independent of de novo protein synthesis.

Active NFκB is a dimer of proteins of the Rel family, which contain a conserved 300 amino acid N-terminal domain known as the Rel homology domain. This region is responsible for DNA binding, for dimerization with other Rel proteins, for nuclear localization and for binding to IκB. Each Rel protein contains one half of the required DNA binding site, thus permitting the appropriate Rel combination to be specified by slight variations in the consensus NFκf binding site, 5′-GGGGYNNCCY-3′.

As indicated above, Rel proteins are bound in the cytoplasm by IκB molecules. IκBs are ankyrin repeat containing molecules, of which a number have been characterized, including IκB-α, β, γ, ε, Bc1-3 and Cactus. Bc1-3 is a polypeptide of higher vertebrates, whilst Cactus is a Drosophila gene. The interaction between the ankyrin repeats and NFκB/Rel appears to be an evolutionarily conserved mechanism for the regulation of NFκB proteins.

The Rel family of proteins includes Relish, Dif, Dorsal, RelB, c-Rel, v-Rel (chicken oncogene), p65, p100/p52 and p105/p50. The first three listed are Drosophila proteins The latter two polypeptides are unusual in that the larger, precursor molecule (p100 or p105) encodes both a Rel protein and an IκB, which combines with its associated Rel protein to block its nuclear localization. As monomers, or homodimers, p50 and p52 do. not contain transcriptional activation domains. Hence, in order to activate gene transcription they associate in the form of heterodimers with another transactivating Rel protein. Homodimers of p50/p52 may repress gene transcription in certain cell types.

Activation of NFκB/Rel is triggered by phosphorylation of IκB. This tags IκB for degradation by the proteosome, but mechanisms for IκB phosphorylation have remained largely unclear to date. In the case of p100/p105, proteolytic cleavage of the C-terminal ankyrin repeat containing region from the Rel region is required, in order to unmask the nuclear localization signal of p52/p50.

In vivo, NFκB plays an important role in the regulation of genes involved in immune, acute phase and inflammatory responses. Although NFκB effects are highly pleiotropic, the effects of p105 have been investigated in knockout mice (p105^{-/-}). In these animals, the C-terminal region of p105 was deleted, such that the mice were capable of expressing p50 but in a form not complexed with the IκB-like inhibitory ankyrin repeats of p105. In other words, constitutively active p50 was produced (Ishikawa et al, (1998) J. Exp. Med. 187:985-996). These mice displayed an inflammatory phenotype, comprising lymphocytic infiltration in the lungs and liver, an increased susceptibility to infection, enlargement of multiple lymph nodes, splenomegaly and lymphoid hyperplasia. The cytokine producing ability of macrophages were impaired, whilst B-cell proliferation was increased.

Inappropriate or incorrect synthesis of NFκB is associated with a variety of diseases and dysfunctions in mammals. For example, as indicated by Schreck et al. (1991) EMBO J. 10:2247-2258, migration of NFκB to the nucleus is associated with transcription of the HIV genome and production of HIV virions in HIV infected cells, as well as HIV gene expression (Swingler et al., (1992) AIDS Res Hum Retroviruses 8:487-493). It is also involved in the replication of other retroviruses, such as EBV (Powell et al.., (1993) Clin Exp Immunol 91:473-481).

Moreover, NFκB is known to protect cells from apoptosis (see e.g. Sikora et al., (1993) BBRC 197:709-715), mediate the biological effects of TNF (Renier et al., (1994) J Lipid Res 35:271-278; WO 97/37016), the response to stress (Tacchini et al., (1995) Biochem J 309:453-459) and protect cells from, for example, ischemia (Mattson, (1997) Neurosci. Biobehav. rev. 21:193-206), and is associated with various cancers (Chang et al., (1994) Oncogene 9:923-933; Enwonwu and Meeks, (1995) Crit Rev Oral Biol Med 6:5-17; Denhardt, (1996) Crit Rev Oncog 7:261-291).

In general, however, NFκB is involved in the regulation of the expression of a large variety of cytokines and lymphokines. This suggests a role for modulators of NFκB activity in the treatment of conditions associated with or involving stress, infection or inflammation, or in the treatment of conditions by employing responses, such as inflammatory responses, which are controlled by NFκB in vivo.

TPL-2 was originally identified, in a C-terminally deleted form, as the product of an oncogene associated with Moloney murine leukemia virus-induced T cell lymphomas in rats (Patriotis, et al., (1993) Proc. Natl. Acad. Sci. USA 90:2251-2255). TPL-2 is a protein serine kinase which is homologous to MAP kinase kinase kinases (3K) in its catalytic domain (Salmeron, A., et al., (1996) EMBO J. 15:817-826) and is >90% identical to the proto-oncogene product of human COT (Aoki, M., (1993) et al. J. Biol. Chem. 268:22723-22732). TPL-2 is also highly homologous to the kinase NIK, which has been shown to regulate the inducible degradation of IκB-α (Malinin et al., (1997) Nature 385:540-544; WO 97/37016; May and Ghosh, (1998) Immunol. Today 19:80-88). However, the biological function of TPL-2/COT has hitherto not been known.

### SUMMARY OF THE INVENTION

The present invention relates to a novel pathway for the regulation of NFκB. In particular, the invention relates to the use of the kinase TPL-2/COT as a target for the development of agents capable of modulating NFκB and, in a preferred embodiment, agents capable of modulating the interaction of the IκB p105 with TPL-2. Throughout the specification, the term “TPL-2” will be understood to include rat TPL-2 and the human TPL-2 homolog COT unless otherwise stated. It is also understood that any TPL-2 homolog, preferably a mammalian TPL-2 homolog, is included within the scope of the invention. The term “NFκB”, unless otherwise defined, is intended to encompass any protein (or fragment thereof), or protein complex having NFκB-binding activity as recognized in the art. Such a protein or protein complex may comprise one or more proteins and take the form of a homodimer, heterodimer, or multimer. Typically, such a complex may comprise, e.g., rel A, rel B, p50, p52, p65, c-Rel, v-Rel, and/or dorsal.

It is shown below that TPL-2 is responsible for degradation of p105 and resultant release of Rel subunits. Accordingly, the invention provides TPL-2 as a specific regulator of the degradation of p105, and thus as a modulator of inflammatory responses in which p50 Rel is involved.

In a first aspect of the present invention, therefore, there is provided the use of TPL-2 in the modulation of NFκB activity such that modulation of NFκB occurs. In a preferred embodiment, modulation occurs via p105.

In a second aspect of the present invention, there is provided a method for identifying a compound or compounds capable, directly or indirectly, of modulating the proteolysis of p105 and thereby its inhibitory activity, comprising the steps of:

(a) incubating a TPL-2 molecule with the compound or compounds to be assessed; and

(b) identifying those compounds which influence the activity of the TPL-2 molecule. As demonstrated below, TPL-2 is found to be responsible for the direct or indirect phosphorylation of p105, which leads directly to its degradation and translocation to the nucleus of the associated Rel subunit, as a homodimer or as a heterodimer with a further Rel monomer. Accordingly, compounds which are capable of modulating the direct or indirect interaction between TPL-2 and p105, either by binding to TPL-2, modulating the activity of TPL-2 or influencing the interaction of TPL-2 with p105 or with other polypeptides involved in the phosphorylation of p105, are capable of modulating the activation of NFκB via p105.

Moreover, the invention provides methods for producing polypeptides capable of modulating TPL-2 activity, including expressing nucleic acid sequences encoding them, methods of modulating NFκB activity in cells in vivo, and methods of treating conditions associated with NFκB or in which it is desirable to induce or repress inflammation.

In a further aspect of the invention, there is provided the use of TPL-2 for the modulation of tumour necrosis factor activity in or on a cell. As set out below, TNF activation of gene transcription may be blocked by the use of a TPL-2 antagonist, TPL-2(A270).

TNF-α is known to be capable of stimulating p105 degradation and NFκB-induced activation of gene transcription. The invention therefore concerns a method for modulating the TNF activation pathway of p105. In a preferred embodiment, the invention provides a method for identifying a lead compound for a pharmaceutical, comprising:

incubating a compound or compounds to be tested with a TPL-2 molecule and tumour necrosis factor (TNF), under conditions in which, but for the presence of the compound or compounds to be tested, the interaction of TNF and TPL-2 induces a measurable chemical or biological effect;

determining the ability of TNF to interact, directly or indirectly, with TPL-2 to induce the measurable chemical or biological effect in the presence of the compound or compounds to be tested; and

selecting those compounds which modulate the interaction of TNF and TPL-2.

In a preferred embodiment, the invention comprises a method for identifying a lead compound for a pharmaceutical, comprising the steps of:

providing a purified TPL-2 molecule;

incubating the TPL-2 molecule with a substrate known to be phosphorylated by TPL-2 and a test compound or compounds; and

identifying the test compound or compounds capable of modulating the phosphorylation of the substrate.

Optionally, the test compound(s) identified may then be subjected to in vivo testing to determine their effects on a TNF/p105 originating signaling pathway.

In another aspect, the invention provides a method for identifying a compound which regulates an inflammatory response mediated by TPL-2 that includes, contacting a reaction mixture that includes a TPL-2 polypeptide, or fragment thereof, with a test compound and determining the effect of the test compound on an indicator of NFκB activity to thereby identify a compound that regulates NFκB activity mediated by TPL-2.

In a related aspect, the invention provides a method for identifying a compound which regulates TPL-2-mediated NFκB activity.

In another aspect, the invention provides a method for identifying a compound which regulates signal transduction by TPL-2 that includes, contacting a reaction mixture containing a TPL-2 polypeptide, or a fragment thereof, with a test compound, and determining the effect of the test compound on an indicator of signal transduction by the TPL-2 polypeptide in the reaction mixture in order to identify a compound which regulates signal transduction by TPL-2.

In even another aspect, the invention provides a method for identifying a compound which modulates the interaction of a TPL-2 polypeptide with a target component of TPL-2 modulation that includes, contacting a reaction mixture containing a TPL-2 polypeptide or fragment thereof, with a target component of the TPL-2 modulation, and a test compound, under conditions where, but for the presence of the test compound, the TPL-2 polypeptide, or fragment thereof, specifically interacts with the target component at a reference level. Accordingly, the method allows for measuring a change in the level of interaction in the presence of the test compound, where a difference indicates that the test compound modulates the interaction of a TPL-2 polypeptide, or fragment thereof, with a target component of TPL-2 modulation. In a preferred embodiment, the target component is p105, IκB-α, IκB-β, MEK-1, SEK-1, or NFκB and preferably, a purified polypeptide.

In a preferred embodiment of the foregoing aspects, the method encompasses the use of a TPL-2 polypeptide, preferably a recombinant polypeptide, that includes an amino acid sequence having at least 75% identity with the amino acid sequence provided in SEQ ID NO: 2 or SEQ ID NO: 4.

In another preferred embodiment of the foregoing aspects, the method encompasses a TPL-2 polypeptide, preferably a recombinant polypeptide, that is encoded by a nucleic acid molecule which hybridizes under highly stringent conditions with a nucleic acid molecule having a sequence provided in SEQ ID NO: 1 or SEQ ID NO: 3.

In another preferred embodiment of the foregoing aspects, the method involves the use of a cell-free mixture or a cell-based mixture and such a mixture may be derived from a recombinant cell, preferably a recombinant cell having a heterologous nucleic acid encoding a TPL-2 polypeptide. In a preferred embodiment, the cell-free mixture may employ a purified TPL-2 polypeptide. In another embodiment, the method includes a determination of signaling that includes TNF expression. In a related embodiment, the recombinant cell includes a reporter gene construct that is operably linked with a transcriptional regulatory sequence sensitive to intracellular signals transduced by TPL-2 or NFκB. In a preferred embodiment, the transcriptional regulatory sequence is a TNF transcriptional regulatory sequence.

In another preferred embodiment of the foregoing aspects, the method includes a determination of TPL-2 activity such as kinase activity, binding activity, and/or signaling activity.

In even another preferred embodiment of the foregoing aspects, the method includes a determination that includes measuring apoptosis of a cell, cell proliferation, or an immune response.

In even another preferred embodiment of the foregoing aspect, the method includes the use of a test compound that is protein based, carbohydrate based, lipid based, nucleic acid based, natural organic based, synthetically derived organic based, or antibody based.

In another preferred embodiment, the invention provides a compound identified according to the method of the foregoing aspects, and preferably, such a compound is suitable for treating a condition such as multiple sclerosis (MS), inflammatory bowel disease (IBD), insulin-dependent diabetes mellitus (IDDM), sepsis, psoriasis, graft rejection, misregulated TNF expression, or, preferably, rheumatoid arthritis.

In another aspect, the invention provides a method for treating an immune system condition in a subject in need thereof by modulating TPL-2 activity by, administering a pharmaceutical composition capable of modulating TPL-2 in an amount sufficient to modulate the immune system response in the patient.

In a related aspect, the invention provides a method for treating a TPL-2-mediated condition in a subject by, administering a composition capable of modulating TPL-2 and in a therapeutically-effective amount sufficient to modulate the TPL-2-mediated condition in the recipient subject.

In another related aspect, the invention provides a method for modulating TPL-2-mediated NFκB regulation in a subject in need thereof by, administering a therapeutically-effective amount of a pharmaceutical composition to the human such that modulation occurs.

In even another related aspect, the invention provides a method for modulating TPL-2-mediated NFκB regulation within a cell including, administering to a cell a composition capable of modulating TPL-2 in an amount sufficient such that a change in TPL-2-mediated NFκB regulation is achieved.

In a preferred embodiment of the foregoing aspects, the condition to be treated is multiple sclerosis (MS), inflammatory bowel disease (IBD), insulin-dependent diabetes mellitus (IDDM), sepsis, psoriasis, graft rejection, misregulated TNF expression, or, preferably, rheumatoid arthritis.

In another preferred embodiment of the foregoing aspects, the composition administered contains a compound selected from the group consisting of N1-[4-(4-amino-7-cyclopentyl-7H-pyrrolo[2,3-d]pyrimidin-5-yl)-2-chlorophenyl]-1-benzenesulfonamide, ethyl 5-oxo-4-[4-(phenylsulfanyl)anilino]-5,6,7,8-tetrahydro-3-quinolinecarboxylate, 3-(4-pyridyl)-4,5-dihydro-2H-benzo[g]indazole methanesulfonate, and sodium 2-chlorobenzo [1][1,9] phenanthroline-7-carboxylate.

In another aspect, the invention provides a method for treating TNF misregulation by, administering to a subject at risk for TNF misregulation a therapeutically-effective amount of a TPL-2 modulator such that treatment occurs.

In a related aspect, the invention provides a method for treating rheumatoid arthritis by, administering to a subject at risk for rheumatoid arthritis a therapeutically-effective amount of a TPL-2 modulator such that treatment occurs.

In a preferred embodiment of the two foregoing aspects, the TPL-2 modulator is N1-[4-(4-amino-7-cyclopentyl-7H-pyrrolo[2,3-d]pyrimidin-5-yl)-2-chlorophenyl]-1-benzenesulfonamide, ethyl 5-oxo-4-[4-(phenylsulfanyl)anilino]-5,6,7,8-tetrahydro-3-quinolinecarboxylate, 3-(4-pyridyl)-4,5-dihydro-2H-benzo[g]indazole methanesulfonate, or sodium 2-chlorobenzo[1][1,9]phenanthroline-7-carboxylate.

In even another embodiment, where the condition being treated is arthritis, e.g., rheumatoid arthritis, a TPL-2 modulator is employed that is not 3-(4-pyridyl)-4,5-dihydro-2H-benzo[g]indazole methanesulfonate.

The present invention further demonstrates a novel and unexpected link between NF-κB and the regulation of MEK activity. Thus, interaction with the death domain of NF-κB1 p105 profoundly inhibits the ability of TPL-2 to phosphorylate MEK when the TPL-2 C-terminus simultaneously binds to p105₄₉₈₋₅₄₁. NF-κB1 p105 is therefore a negative regulator of TPL-2 MEK kinase, in addition to its functions as precursor for p50 and an IκB protein (Karin, M., and Y. Ben-Neriah. (2000) Annu. Rev. Immunol. 18:621-663). Discovery that a C-terminal truncation renders TPL-2 relatively insensitive to p105 negative regulation, reveals why this mutation is oncogenic (Ceci, J. D., et al. (1997) Gene. Devel. 11:688-700).

One aspect of this invention is directed to a composition of matter comprising a first nucleic acid comprising a first nucleic acid sequence encoding a first polypeptide; and a second nucleic acid comprising a second nucleic acid sequence encoding a second polypeptide, wherein the first polypeptide comprises TPL-2 kinase, and the second polypeptide comprises p105. Preferably, the composition of matter further comprises a first expression vector; and a second expression vector, wherein the first expression vector comprises the first nucleic acid and the second expression vector comprises the second nucleic acid. More preferably, the composition of matter comprises an expression vector, wherein the expression vector comprises the first nucleic acid and the second nucleic acid.

In another embodiment the first nucleic acid is operably linked to a first promoter and the second nucleic acid is operably linked to a second promoter. More preferably, the first nucleic acid and the second nucleic acid are operably linked to one promoter. Preferably the promoters are selected from the group consisting of retroviral promoters, viral promoters, bacterial promoters, baculoviral promoters, and eukaryotic promoters. More preferably, the promoters are baculoviral polyhedrin promoter and a baculoviral p10 promoter.

In a preferred embodiment, the TPL-2 kinase encoded by the first polypeptide is any one of TPL-2 (30-467), TPL-2 (30-397), TPL-2 (104-397), TPL-2 (131-397), TPL-2 (131-411), TPL-2 (1-467), TPL-2 (1-467, M30V), TPL-2 (30-467, D253N), TPL-2 (30-467, S368A), TPL-2 (30-467, D253N, S368A), TPL-2 (1-467, M30V, D253N), and TPL-2 (1-467, M30V, D253N, S368A).

In another preferred embodiment the p105 encoded by the second polypeptide is selected from the group consisting of p105 (369-969), p105 (369-896), p105 (400-896), p105 (444-896), p105 (472-896), p105 (472-896, S894A), p105 (1-969, Δ803-893), p105 (1-969, Δ498-541), p105 (1-969, L842A), p105(498-806), p105(498-782), and p105(809-893).

In a further embodiment a nucleic acid sequence encoding a detectable tag is operably linked to at least one or both of the first nucleic acid and the second nucleic acid. Preferably the detectable tag is selected from the group consisting of GFP, HA, myc, FLAG, 6HIS, maltose binding protein and GST. More preferably the detectable tag operably linked to at least one or both of the first nucleic acid and the second nucleic acid is FLAG or HA. Most preferably, the nucleic acid sequence encoding the detectable tag FLAG is operably linked to the first nucleic acid and nucleic acid sequence encoding HA is operably linked to the second nucleic acid.

The most preferred embodiment of the invention is a composition of matter comprising an expression vector which comprises a first nucleic acid sequence encoding TPL-2(30-467) operably linked to a FLAG tag, and a second nucleic acid sequence encoding p105(369-896) operably linked to an HA tag, wherein said first nucleic acid is under the control of baculovirus polyhedrin promoter and said second nucleic acid is under the control of the baculoviral p10 promoter.

In another embodiment, a host cell is transformed with the composition of matter described above. Preferably the host cell is an eukaryotic cell selected from the group consisting of a mammalian cell, fungal cell, avian cell, plant cell, and insect cell.

Most preferably the host cell is the insect cell line SF9 or the mammalian cell line HEK 293. In another embodiment the host cell is a prokaryotic cell such as E.Coli.

The present invention also provides a method for producing TPL-2 kinase comprising the steps of (a) providing a host cell transformed with an expression vector of the invention; and (b) culturing the host cell under conditions sufficient to allow expression of TPL-2 kinase. In a further embodiment the method comprises additional steps of (c) lysing said host cell; and (d) isolating TPL-2 kinase.

The present invention also provides a method for producing p105 comprising the steps of (a) providing a host cell transformed with an expression vector of the invention; and (b) culturing the host cell under conditions sufficient to allow expression of p105. In a further embodiment the method comprises additional steps of (c) lysing said host cell; and (d) isolating p105.

The present invention also provides a method for producing TPL-2/p105 complex comprising the steps of (a) providing a host cell transformed with an expression vector of the invention; and (b) culturing the host cell under conditions sufficient to allow expression of TPL-2/p105 complex. In a further embodiment the method comprises additional steps of (c) lysing said host cells; and (d) isolating TPL-2/p105 complex.

Another embodiment provides a method for isolating TPL-2 kinase comprising the steps of (a) producing TPL-2/p105 complex as stated above; (b) adding 8M urea sufficient to disrupt TPL-2/p105 complex; and (c) isolating TPL-2 kinase. Yet another embodiment provides a method for isolating p105 kinase comprising the steps of (a) producing TPL-2/p105 complex as stated above; (b) adding 8M urea sufficient to disrupt TPL-2/p105 complex; and (c) isolating p105.

The most preferred embodiment pertains to TPL-2 kinase, p105 and TPL-2/p105 complex produced according to any one of the methods described above.

### BRIEF DESCRIPTION OF THE DRAWINGS

FIG. 1

TPL-2 C-terminus is required for interaction with NF-κB1 p105 in vitro.

A) TPL-2 deletion mutants. Positions of myc and TSP3 epitopes (Salmeron, A., et al., (1996) EMBO J. 15, 817-826) are indicated. M30 corresponds to the alternative initiation site of TPL-2 (Aoki, M., et al., (1993) J. Biol. Chem. 268, 22723-22732). B) TPL-2ΔC does not form a stable complex with p105. p105 (Blank, et al., (1991) EMBO J. 10, 41594167) is synthesized and labeled with [³⁵S]-Met by in vitro cell-free translation on its own or together with either TPL-2 or TPL-2ΔC (Salmeron, et al., 1996). The appropriate translation mixes are then immunoprecipitated with anti-TPL-2 antibody −/+ competing peptide. Isolated proteins are resolved by 10% SDS-PAGE and revealed by fluorography (right hand panel). Left panel, labeled lysates', shows TPL-2 and p105 expression in the entire rabbit reticulocyte lysate translation mix. p105 translated in vitro generated low levels of p50 (lane 3) which are only visible on over-exposure of the film (data not shown). C) The TPL-2 N-terminus is not required for binding to p105. The indicated TPL-2 proteins (all myc epitope-tagged at their N-terminus) are translated in vitro with p105 as in B and then immunoprecipitated with anti-myc MAb. [³⁵S]-Met-labeled proteins are revealed by fluorography after 10% SDS-PAGE. Lower panel shows p105 expression in Lysates.

FIG. 2

TPL-2 interacts with the C-terminus of NF-κB1 p105 in vitro.

A) p105 deletion mutants (Fan, et al., (1991) Nature 354, 395-398). Positions of Rel homology domain (RHD) (Ghosh, et al., (1998) Annu. Rev. Immunol. 16, 225-260), glycine rich region (GRR) (Lin, et al., (1996) Mol. Cell. Biol. 16, 2248-2254) and antibody epitopes, myc and NF-κB1(N), are shown. The open arrowhead shows the position of the p50 C-terminus. The closed arrowheads show N-terminal start sites of the various TPL-2-interacting NF-κB1 two-hybrid clones, which all continued to the C-terminal end of the protein. B) and C) TPL-2 interacts with the C-terminus of p105. TPL-2 is translated in vitro together with the indicated p105 mutants. Complex formation is analyzed by 8% SDS-PAGE of anti-TPL-2 immunoprecipitates (right panels) and fluorography. Left panels show expression of TPL-2 and p105 mutants in Lysates. The arrowhead in B) indicates the position of p48.

FIG. 3

TPL-2 is associated with NF-κB1 p105 in vivo and activates an NF-κB-dependent reporter gene after transient expression.

A) TPL-2 is associated with p105 in vivo. HeLa cell lysates are immunoprecipitated with the indicated antibodies −/+ competing peptide. Isolated proteins are resolved by 10% SDS-PAGE and then sequentially western blotted for the proteins shown. B) The majority of TPL-2 is complexed with p 105 in vivo. HeLa cell lysate is serially immunoprecipitated with anti-NF-κB1 antibody three times. Western blotting of cell Lysates confirmed depletion of p105/p50, but not of α-tubulin. TPL-2 content of NF-κB1-depleted Lysates is determined by probing western blots of Lysates, and of anti-TPL-2 immunoprecipitates from Lysates, with anti-TPL-2 antiserum. C) TPL-2 expression activates an NF-κB-dependent luciferase reporter gene. Jurkat T cells are transfected with 0.5 μg of the indicated expression vectors plus 2 μg of the reporter construct (total DNA is adjusted to 4 μg with empty pcDNA3 vector). Luciferase assays are done in duplicate and are expressed as a mean stimulation index relative to empty vector control (+/−SE). TPL-2ΔC data are normalized based on its expression level, determined by western blotting, relative to TPL-2, which is assigned an arbitrary value of 1. TPL-2(A270) is a kinase-inactive point mutant of TPL-2. D) Co-expression of a C-terminal p105 fragment blocks NF-κB activation by TPL-2. Jurkat T cells are transfected with 0.5 μg of the indicated expression vectors and either 2 μg of empty vector or the 3′NN construct plus 2 μg of NF-κB luciferase reporter construct. Duplicate luciferase assays are expressed as a mean stimulation index relative to empty vector control (+/−SE). Western blotting confirmed that expression of 3′NN did not affect the expression of co-transfected TPL-2 (data not shown).

FIG. 4

Co-expression of TPL-2 with myc-p105 induces nuclear translocation of active mycp50.

A) TPL-2 induces nuclear translocation of co-expressed NF-κB 1.3T3 cells are transiently transfected with 0.5 μg each of the indicated expression vectors and stained for indirect immunofluorescence using anti-myc MAb (green) to localize myc-p105/myc-p50 and anti-TPL-2 antiserum (red). Images shown are single confocal sections through representative transfected cells. Phase contrast images are also shown.

B) TPL-2 induces myc-p50 to translocate into the nucleus. Cytoplasmic and nuclear extracts are prepared from cells transfected with the indicated vectors. Myc-p105/myc-p50 are revealed by probing western blots of anti-myc immunoprecipitates with anti-NF-κB1(N) antiserum. Comparison with total cell Lysates suggested that myc-p50 is inefficiently extracted from the nuclear fraction and is, therefore, underrepresented.

C) Nuclear NF-κB 1 induced by TPL-2 is biologically active. NF-κB DNA-binding activity of nuclear extracts, prepared from 3T3 cells transfected with the indicated expression vectors (0.5 μg each; Watanabe, et al., (1997) EMBO J. 16, 3609-3620), is analyzed by EMSA (Alkalay, I., et al. (1995) Mol. Cell Biol. 15, 1294-1301). Closed arrowheads show the position of the two detected NF-κB complexes. Open arrowheads show the position of antibody-supershifted NF-κB complexes (lanes 6 and 7). In lane 8, competition with 100-fold unlabelled κB oligonucleotide demonstrated the specificity of detected NF-κB complexes.

FIG. 5

TPL-2 promotes nuclear translocation of p50 independently of p105 processing.

3T3 cells are transiently transfected with vectors encoding HA-p50 (0.4 μg), either TPL-2(A270) or TPL-2 (0.2 μg) and myc-p105AGRR or empty vector (0.4 μg). After 24 h in culture, cells are stained for indirect immunofluorescence using anti-HA MAb to localize HA-p50 (green) and anti-TPL-2 antiserum (red). Images shown are single confocal sections through representative transfected cells. Phase contrast images are also presented.

FIG. 6

TPL-2 stimulates proteolysis of co-expressed myc-p105.

A) Effect of TPL-2 co-expression on p105 proteolysis. 3T3 cells are transiently transfected with expression vectors encoding myc-p105 and TPL-2 (TPL-2) or with myc-p105 and empty vector (control). After 24 h in culture, cells are metabolically pulse-labeled with [³⁵S]-Met/[³⁵S]-Cys for 30 min and then chased for the times indicated. Labeled proteins are immunoprecipitated from cell lysates using anti-myc MAb, resolved by 8% SDS-PAGE and revealed by fluorography. Closed arrowheads show position of co-immunoprecipitating TPL-2. Open arrowheads indicate the shift in electrophoretic mobility of myc-p105 caused by TPL-2 co-expression. B) and C) Immunoprecipitated myc-p105 and myc-p50 in panel A are quantified by laser densitometry and data are presented graphically to show the turnover of myc-p105 (B) and the ratio of myc-p50/myc-p105 (C). D) 3T3 cells are transiently transfected with vectors encoding myc-p105AGRR and TPL-2 (TPL-2) or myc-p105AGRR and no insert (control). myc-p105 turnover is determined as in B. E) 3T3 cells are transfected with a vector encoding myc-p105 together with a vector encoding TPL-2(A270) or empty vector (control). Turnover of myc-p105 is determined as in B. F) TPL-2-induced p105 proteolysis is blocked by an inhibitor of the proteasome. 3T3 cells are transfected as in A. MG132 proteasome inhibitor (2011M) or DMSO vehicle (control) is added prior to pulse-labeling and maintained throughout the chase period. Labeled myc-p105 is isolated by immunoprecipitation as in A and quantified by laser densitometry. Data are presented graphically to show the effect of the drug on TPL-2-induced myc-p105 proteolysis. MG132 treatment completely blocked the production of myc-p50 during the chase in TPL-2 co-transfected cells (data not shown). G) 3T3 cells are transfected with the indicated vectors as in A, in duplicates. Steady state levels of myc-p50/myc-p105 are determined after 24 h by probing western blots of cell Lysates with anti-myc antiserum. TPL-2 co-transfection increased the absolute levels of myc-p50 compared to control. Thus myc-p50 may be more stable in TPL-2 co-expressing cells, perhaps due to its nuclear location, since the overall rate of myc-p50 production from myc-p105 is not increased (FIG. 6A).

FIG. 7

TPL-2 activity is required for TNF-α-induced degradation of p105.

A) Kinase-inactive TPL-2 blocks p105 degradation induced by TNF-α. Jurkat T cells are transfected to stably express kinase-inactive TPL-2(A270), as determined by western blotting. Vector control cells, which are stably transfected with empty vector, and two independently derived clones expressing TPL-2(A270), are metabolically pulse-labeled with [³⁵S]-Met/[³⁵S]-Cys for 30 min and then chased for the times indicated in the presence of TNF-a (20 ng/ml) or control medium, as indicated. Labeled p105 is immunoprecipitated from cell Lysates using anti-NF-κB1(N) antiserum, resolved by SDS-PAGE and revealed by fluorography. Immunoprecipitated p105 is quantified by laser densitometry and data are presented in graphical-form.

B) TPL-2 induces phosphorylation of co-expressed myc-p105. 3T3 cells are transiently transfected with vectors encoding myc-p105 and the indicated proteins or no insert control (O). Myc-p105 is isolated by immunoprecipitation with anti-myc MAb and then treated with control buffer (1), phosphatase (2) or phosphatase plus phosphatase inhibitors (3). Isolated protein is resolved by 8% SDS-PAGE and western blotted with anti-NF-κB1(N) antiserum. Arrowheads indicate the shift in electrophoretic mobility of myc-p105 caused by TPL-2 co-expression.

FIG. 8

Dominant negative TPL-2 modulates transcription of TNF-induced reporter gene.

Jurkat T cells are transformed according to the procedure of FIG. 7A, with a vector expressing a luciferase reporter gene under the control of a TNF-inducible NFκB responsive promoter system. Co-expression of TPL-2 KD (kinase dead) or TPL-2 Cter (C-terminal truncation) leads to a decrease in TNF-mediated activation.

FIG. 9

The chemical structure of the compound N-(6-phenoxy-4-quinolyl)-N-[4-(phenylsulfanyl) phenyl] amine is depicted which can inhibit TPL-2 kinase activity by 50% at a level of 50 μM.

FIG. 10

The chemical structure of the compound ethyl 5-oxo-4-[4-(phenylsulfanyl)anilino]-5,6,7,8-tetrahydro-3-quinolinecarboxylate is depicted which can inhibit TPL-2 kinase activity by 50% at a level of 10 μM.

FIG. 11

The chemical structure of the compound 3-(4-pyridyl)-4,5-dihydro-2H-benzo[g]indazol-2-ium methanesulfonate is depicted which can inhibit TPL-2 kinase activity by 50% at a level of 100 μM.

FIG. 12

The chemical structure of the compound sodium 2-chlorobenzo[l][1,9]phenanthroline-7-carboxylate is depicted which can inhibit TPL-2 kinase activity by 50% at a level of 100 μM.

FIG. 13

An autoradiograph is shown that demonstrates the inhibitory activity of several different compounds in reducing the level of TPL-2 autophosphorylation (FLAG-COT (30-397) and phosphorylation of a target polypeptide, i.e., GST-IκB-α (Lane 1, 3-(4-pyridyl)-4,5-dihydro-2H-benzo[g] indazole; Lane 2, ethyl 5-oxo-4-[4-(phenylsulfanyl)anilino]-5,6,7,8-tetrahydro-3-quinolinecarboxylate; Lane 3, N-(6-phenoxy-4-quinolyl)-N-[4-(phenylsulfanyl)phenyl]amine; Lane 4, staurosporin; Lane 5, SB 203580; Lane 6, PD 098059; Lane 7, FLAG-COT (30-397) and vehicle only (DMSO); and Lane 8, FLAG-COT (30-397), GST-IκB-α, and vehicle only (DMSO); see text for further details).

FIG. 14

The core structure of quinolinyl derivatives is depicted.

### DETAILED DESCRIPTION OF THE INVENTION

TPL-2 (tumour progression locus 2) is a MAP kinase kinase kinase first isolated in association with a Moloney murine leukemia virus. The gene (tpl-2) encodes a polypeptide which is associated with tumour progression and tumorigenesis in a variety of systems, and which appears to be activated in tumors by C-terminal truncation (Makris et al., (1993) J Virol 67:1286-1291; Patrotis et al., (1993) PNAS (USA) 90:2251-2255; Makris et al., (1993) J Virol 67:4283-4289; Patrotis et al., (1994) PNAS (USA) 91:97559759; Salmeron et al., (1996) EMBO J 15:817-826; Ceci et al., (1997) Genes Dev 11:688-700). The complete nucleic acid and amino acid sequences of rat TPL-2 are available in GenBank under accession number M94454. The nucleic acid and amino acid sequences of the human TPL-2 homolog termed COT, for cancer Osaka thyroid, are available in GenBank under accession numbers NM_{—}005204 and 729884 (see also, e.g., Miyoshi, et al., Mol. Cell. Biol. 11 (8), 4088-4096 (1991))

1. TPL-2 is a NFκB Regulator

In a first aspect, the invention relates to the use of a TPL-2 molecule for the modulation of NFκB activity.

la. Uses of the TPL-2 Molecule

The invention includes, for example, the use of TPL-2 molecules to modulate NFκB activity in in vitro and/or in vivo assays, and in particular to phosphorylate p105 in such assay systems; the use of a TPL-2 molecule to modulate NFκB activity in a cell in vivo, for example in order to induce or prevent an immune reaction or an inflammatory response. In an advantageous embodiment, the invention relates to the use of a TPL-2 molecule in the treatment of a disease associated with deregulated NFκB expression.

In a preferred embodiment, the TPL-2 molecule according to the invention is useful for modulating the transcription of genes under the control of the NFκB control element, either in vivo, or, for example in an assay method conducted in vitro or in cells, such as in cell culture.

A TPL-2 molecule for use in-an assay or method as defined above may be designed to induce or prevent p105 phosphorylation and proteolysis. Thus, for example, a TPL-2 molecule having the biological activity of wild-type TPL-2 and able to bind to and phosphorylate p105 may be used to induce p105 degradation and /or an inflammatory response. Moreover, a constitutively active mutant of TPL-2 may be used, thus divorcing the activity thereof from further cellular control pathways.

In a further aspect of the invention, a “kinase dead” dominant negative mutant of TPL-2 may be used to down regulate p105 phosphorylation, by competing with endogenous wild-type TPL-2 for p105 but failing to regulate phosphorylation of the target. A kinase dead mutant is preferably prepared by mutating TPL-2 in the kinase domain, for example at position 270. Mutations may be performed at random and selected by assessment of the ability to phosphorylate an artificial substrate or may be designed by modeling of the active site and site-specific mutagenesis to prevent or reduce kinase activity. Preferred kinase dead mutants are TPL-2(A270) and TPL-2(R167). Both of these known mutants were predicted from sequence homologies in the structure of TPL-2.

lb. The TPL-2 Molecule

As used herein, “a TPL-2 molecule” refers to a polypeptide having at least one biological activity of TPL-2. The term thus includes fragments of TPL-2 which retain at least one structural determinant of TPL-2.

The preferred TPL-2 molecule has the structure set forth in GenBank (Accession No. M94454). This polypeptide, rat TPL-2, is encoded by the nucleic acid sequence also set forth under accession no M94454. Alternative sequences encoding the polypeptide of M94454 may be designed, having regard to the degeneracy of the genetic code, by persons skilled in the art. Moreover, the invention includes TPL-2 polypeptides which are encoded by sequences which have substantial homology to the nucleic acid sequence set forth in M94454. “Substantial homology”, where homology indicates sequence identity, means more than 40% sequence identity, preferably more than 45% sequence identity, preferably more than 55% sequence identity, preferably more than 65% sequence identity, and most preferably a sequence identity of 75% or more, as judged by direct sequence alignment and comparison.

For example, the term “a TPL-2 molecule” refers to COT, the human homologue of TPL-2 (Accession No. NM 005204). COT is 90% identical to TPL-2.

Sequence homology (or identity) may moreover be determined using any suitable homology algorithm, using for example default parameters. Advantageously, the BLAST algorithm is employed, with parameters set to default values. The BLAST algorithm is described in detail at http:/Hwww.nchi.nih.gov/BLAST/blast_help.html, which is incorporated herein by reference. The search parameters are defined as follows, and are advantageously set to the defined default parameters.

Advantageously, “substantial homology” when assessed by BLAST equates to sequences which match with an EXPECT value of at least about 7, preferably at least about 9 and most preferably 10 or more. The default threshold for EXPECT in BLAST searching is usually 10.

BLAST (Basic Local Alignment Search Tool) is the heuristic search algorithm employed by the programs blastp, blastn, blastx, tblastn, and tblastx; these programs ascribe significance to their findings using the statistical methods of Karlin and Altschul (see http:H/www.ncbi.nih.gov/BLAST/blast_help.html) with a few enhancements. The BLAST programs were tailored for sequence similarity searching, for example to identify homologues to a query sequence. The programs are not generally useful for motif-style searching. For a discussion of basic issues in similarity searching of sequence databases, see Altschul et al. (1994) Nature Genetics 6:119-129.

The five BLAST programs available at http:H/www.ncbi.nlm.nih.gov perform the following tasks:

blastp compares an amino acid query sequence against a protein sequence database;

blastn compares a nucleotide query sequence against a nucleotide sequence database;

blastx compares the six-frame conceptual translation products of a nucleotide query sequence (both strands) against a protein sequence database;

tblastn compares a protein query sequence against a nucleotide sequence database dynamically translated in all six reading frames (both strands).

tblastx compares the six-frame translations of a nucleotide query sequence against the six-frame translations of a nucleotide sequence database.

BLAST uses the following search parameters:

HISTOGRAM Display a histogram of scores for each search; default is yes. (See parameter H in the BLAST Manual).

DESCRIPTIONS Restricts the number of short descriptions of matching sequences reported to the number specified; default limit is 100 descriptions. (See parameter V in the manual page). See also EXPECT and CUTOFF.

ALIGNMENTS Restricts database sequences to the number specified for which high-scoring segment pairs (HSPs) are reported; the default limit is 50. If more database sequences than this happen to satisfy the statistical significance threshold for reporting (see EXPECT and CUTOFF below), only the matches ascribed the greatest statistical significance are reported. (See parameter B in the BLAST Manual).

EXPECT The statistical significance threshold for reporting matches against database sequences; the default value is 10, such that 10 matches are expected to be found merely by chance, according to the stochastic model of Karlin and Altschul (1990). If the statistical significance ascribed to a match is greater than the EXPECT threshold, the match will not be reported. Lower EXPECT thresholds are more stringent, leading to fewer chance matches being reported. Fractional values are acceptable. (See parameter E in the BLAST Manual).

CUTOFF Cutoff score for reporting high-scoring segment pairs. The default value is calculated from the EXPECT value (see above). HSPs are reported for a database sequence only if the statistical significance ascribed to them is at least as high as would be ascribed to a lone HSP having a score equal to the CUTOFF value. Higher CUTOFF values are more stringent, leading to fewer chance matches being reported. (See parameter S in the BLAST Manual). Typically, significance thresholds can be more intuitively managed using EXPECT.

MATRIX Specify an alternate scoring matrix for BLASTP, BLASTX, TBLASTN and TBLASTX. The default matrix is BLOSUM62 (Henikoff & Henikoff, 1992). The valid alternative choices include: PAM40, PAM120, PAM250 and IDENTITY. No alternate scoring matrices are available for BLASTN; specifying the MATRIX directive in BLASTN requests returns an error response.

STRAND Restrict a TBLASTN search to just the top or bottom strand of the database sequences; or restrict a BLASTN, BLASTX or TBLASTX search to just reading frames on the top or bottom strand of the query sequence.

FILTER Mask off segments of the query sequence that have low compositional complexity, as determined by the SEG program of Wootton & Federhen (1993) Computers and Chemistry 17:149-163, or segments consisting of short-periodicity internal repeats, as determined by the XNU program of Claverie & States (1993)

Computers and Chemistry 17:191-201, or, for BLASTN, by the DUST program of Tatusov and Lipman (see http://www.nchi.nlm.nih.gov). Filtering can eliminate statistically significant but biologically uninteresting reports from the blast output (e.g., hits against common acidic-, basic- or proline-rich regions), leaving the more biologically interesting regions of the query sequence available for specific matching against database sequences.

Low complexity sequence found by a filter program is substituted using the letter “N” in nucleotide sequence (e.g., “NNNNNNNNNNNNN”) and the letter “X” in protein sequences (e.g., “XXXXXXXXX”).

Filtering is only applied to the query sequence (or its translation products), not to database sequences. Default filtering is DUST for BLASTN, SEG for other programs.

It is not unusual for nothing at all to be masked by SEG, XNU, or both, when applied to sequences in SWISS-PROT, so filtering should not be expected to always yield an effect. Furthermore, in some cases, sequences are masked in their entirety, indicating that the statistical significance of any matches reported against the unfiltered query sequence should be suspect.

NCBI-gi Causes NCBI gi identifiers to be shown in the output, in addition to the accession and/or locus name.

Most preferably, sequence comparisons are conducted using the simple BLAST search algorithm provided at http://www.ncbi.nlm.nih.gov/BLAST.

The invention moreover encompasses polypeptides encoded by nucleic acid sequences capable of hybridizing to the nucleic acid sequence set forth in GenBank M94454 at any one of low, medium or high stringency.

Stringency of hybridization refers to conditions under which polynucleic acids hybrids are stable. Such conditions are evident to those of ordinary skill in the field. As known to those of skill in the art, the stability of hybrids is reflected in the melting temperature (Tm) of the hybrid which decreases approximately 1 to 1.5° C. with every 1% decrease in sequence homology. In general, the stability of a hybrid is a function of sodium ion concentration and temperature. Typically, the hybridization reaction is performed under conditions of higher stringency, followed by washes of varying stringency.

As used herein, high stringency refers to conditions that permit hybridization of only those nucleic acid sequences that form stable hybrids in 1 M Na+ at 65-68° C. High stringency conditions can be provided, for example, by hybridization in an aqueous solution containing 6×SSC, 5×Denhardt's, 1% SDS (sodium dodecyl sulphate), 0.1 Na+pyrophosphate and 0.1 mg/ml denatured salmon sperm DNA as non specific competitor. Following hybridization, high stringency washing may be done in several steps, with a final wash (about 30 min) at the hybridization temperature in 0.2-0.1×SSC, 0.1% SDS.

Moderate stringency refers to conditions equivalent to hybridization in the above described solution but at about 60-62° C. In that case the final wash is performed at the hybridization temperature in 1×SSC, 0.1% SDS.

Low stringency refers to conditions equivalent to hybridization in the above described solution at about 50-52° C. In that case, the final wash is performed at the hybridization temperature in 2×SSC, 0.1% SDS.

It is understood that these conditions may be adapted and duplicated using a variety of buffers, e.g. formamide-based buffers, and temperatures. Denhardt's solution and SSC are well known to those of skill in the art as are other suitable hybridization buffers (see, e.g. Sambrook, et al., eds. (1989) Molecular Cloning: A Laboratory Manual, Cold Spring Harbor Laboratory Press, New York or Ausubel, et al., eds. (1990) Current Protocols in Molecular Biology, John Wiley & Sons, Inc.). Optimal hybridization conditions have to be determined empirically, as the length and the GC content of the probe also play a role.

Advantageously, the invention moreover provides nucleic acid sequence which are capable of hybridizing, under stringent conditions, to a fragment of the nucleic acid sequence set forth in GenBank M94454 or NM 005204 (see, respectively, SEQ ID NO: 1 and SEQ ID NO: 3). Preferably, the fragment is between 15 and 50 bases in length. Advantageously, it is about 25 bases in length.

Given the guidance provided herein, the nucleic acids of the invention are obtainable according to methods well known in the art. For example, a DNA of the invention is obtainable by chemical synthesis, using polymerase chain reaction (PCR) or by screening a genomic library or a suitable cDNA library prepared from a source believed to possess TPL-2 and to express it at a detectable level.

Chemical methods for synthesis of a nucleic acid of interest are known in the art and include triester, phosphite, phosphoramidite and H-phosphonate methods, PCR and other autoprimer methods as well as oligonucleotide synthesis on solid supports. These methods may be used if the entire nucleic acid sequence of the nucleic acid is known, or the sequence of the nucleic acid complementary to the coding strand is available. Alternatively, if the target amino acid sequence is known, one may infer potential nucleic acid sequences using known and preferred coding residues for each amino acid residue.

An alternative means to isolate the gene encoding TPL-2 is to use PCR technology as described e.g. in section 14 of Sambrook et al., 1989. This method requires the use of oligonucleotide probes that will hybridize to TPL-2 nucleic acid. Strategies for selection of oligonucleotides are described below.

Libraries are screened with probes or analytical tools designed to identify the gene of interest or the protein encoded by it. For cDNA expression libraries suitable means include monoclonal or polyclonal antibodies that recognize and specifically bind to TPL-2; oligonucleotides of about 20 to 80 bases in length that encode known or suspected TPL-2 cDNA from the same or different species; and/or complementary or homologous cDNAs or fragments thereof that encode the same or a hybridizing gene. Appropriate probes for screening genomic DNA libraries include, but are not limited to oligonucleotides, cDNAs or fragments thereof that encode the same or hybridizing DNA; and/or homologous genomic DNAs or fragments thereof.

A nucleic acid encoding TPL-2 may be isolated by screening suitable cDNA or genomic libraries under suitable hybridization conditions with a probe, i.e. a nucleic acid disclosed herein including oligonucleotides derivable from the sequences set forth in GenBank accession No. M94454 or NM 005204 (see, respectively, SEQ ID NO: 1 and SEQ ID NO: 3). Suitable libraries are commercially available or can be prepared e.g. from cell lines, tissue samples, and the like.

As used herein, a probe is e.g. a single-stranded DNA or RNA that has a sequence of nucleotides that includes between 10 and 50, preferably between 15 and 30 and most preferably at least about 20 contiguous bases that are the same as (or the complement of) an equivalent or greater number of contiguous bases set forth in M94454. The 20 nucleic acid sequences selected as probes should be of sufficient length and sufficiently unambiguous so that false positive results are minimized. The nucleotide sequences are usually based on conserved or highly homologous nucleotide sequences or regions of TPL-2. The nucleic acids used as probes may be degenerate at one or more positions. The use of degenerate oligonucleotides may be of particular importance where a library is screened from a species in which preferential codon usage in that species is not known.

Preferred regions from which to construct probes include 5′ and/or 3′ coding sequences, sequences predicted to encode ligand binding sites, and the like. For example, either the full-length cDNA clone disclosed herein or fragments thereof can be used as probes. Preferably, nucleic acid probes of the invention are labeled with suitable label means for ready detection upon hybridization. For example, a suitable label means is a radiolabel. The preferred method of labeling a DNA fragment is by incorporating α-32P dATP with the Klenow fragment of DNA polymerase in a random priming reaction, as is well ′Known in the art. Oligonucleotides are usually end-labeled with α-32P-labelled ATP and polynucleotide kinase. However, other methods (e.g. non-radioactive) may also be used to label the fragment or oligonucleotide, including e.g. enzyme labeling, fluorescent labeling with suitable fluorophores and biotinylation.

After screening the library, e.g. with a portion of DNA including substantially the entire TPL-2-encoding sequence or a suitable oligonucleotide based on a portion of said DNA, positive clones are identified by detecting a hybridization signal; the identified clones are characterized by restriction enzyme mapping and/or DNA sequence analysis, and then examined, e.g. by comparison with the sequences set forth herein, to ascertain whether they include DNA encoding a complete TPL-2 (i.e., if they include translation initiation and termination codons). If the selected clones are incomplete, they may be used to rescreen the same or a different library to obtain overlapping clones. If the library is genomic, then the overlapping clones may include exons and introns. If the library is a cDNA library, then the overlapping clones will include an open reading frame. In both instances, complete clones may be identified by comparison with the DNAs and deduced amino acid sequences provided herein.

“Structural determinant” means that the derivative in question retains at least one structural feature of TPL-2. Structural features include possession of a structural motif that is capable of replicating at least one biological activity of naturally occurring TPL-2 polypeptide. Thus TPL-2 as provided by the present invention includes splice variants encoded by mRNA generated by alternative splicing of a primary transcript, amino acid mutants, glycosylation variants and other covalent derivatives of TPL-2 which retain at least one physiological and/or physical property of TPL-2. Exemplary derivatives include molecules wherein the protein of the invention is covalently modified by substitution, chemical, enzymatic, or other appropriate means with a moiety other than a naturally occurring amino acid. Such a moiety may be a detectable moiety such as an enzyme or a radioisotope. Further included are naturally occurring variants of TPL-2 found with a particular species, preferably a mammal. Such a variant may be encoded by a related gene of the same gene family, by an allelic variant of a particular gene, or represent an alternative splicing variant of the TPL-2 gene.

It has been observed that the C-terminus of TPL-2 is necessary for interaction with p105. Thus, the TPL-2 molecule according to the invention preferably retains the C-terminal portion of naturally occurring TPL-2. Preferably, the TPL-2 molecule according to the present invention retains at least amino acids 398-468 of naturally occurring TPL-2, for example TPL-2 as represented in M94454.

Advantageously, the TPL-2 molecule according to the invention comprises amino acids 350-468 of TPL-2; preferably amino acids 300-468 of TPL-2; preferably amino acids 250468 of TPL-2; preferably amino acids 200-468 of TPL-2; and most preferably amino acids 131-468 of TPL-2.

Alternatively, the TPL-2 molecule according to the invention comprises at least one of the seven exons of TPL-2 as shown in M94454. Preferably, therefore, the TPL-2 molecule includes amino acids 425 to 468 (Exon 7); advantageously it includes amino acids 343-424 (Exon 6); preferably, in includes amino acids 256-342 (Exons 5); preferably, it includes amino acids 169 to 255 (Exon 4); preferably, it includes amino acids 113 to 168 (Exon 3); preferably, it includes amino acids 1 to 112 (Exon 2); or any combination of the above.

Moreover, the invention extends to homologues of such fragments as defined above.

Derivatives which retain common structural determinants can, as indicated above, be fragments of TPL-2. Fragments of TPL-2 comprise individual domains thereof, as well as smaller polypeptides derived from the domains. Preferably, smaller polypeptides derived from TPL-2 according to the invention define a single functional domain which is characteristic of TPL-2. Fragments may in theory be almost any size, as long as they retain one characteristic of TPL-2. Preferably, fragments will be between 4 and 300 amino acids in length. Longer fragments are regarded as truncations of the full-length TPL-2 and generally encompassed by the term “TPL-2”.

Derivatives of TPL-2 also comprise mutants thereof, which may contain amino acid deletions, additions or substitutions, subject to the requirement to maintain at least one feature characteristic of TPL-2. Thus, conservative amino acid substitutions may be made substantially without altering the nature of TPL-2, as may truncations from the N terminus. Deletions and substitutions may moreover be made to the fragments of TPL-2 comprised by the invention. TPL-2 mutants may be produced from a DNA encoding TPL-2 which has been subjected to in vitro mutagenesis resulting e.g. in an addition, exchange and/or deletion of one or more amino acids. For example, substitutional, deletional or insertional variants of TPL-2 can be prepared by recombinant methods and screened for immuno-crossreactivity with the native forms of TPL-2.

The fragments, mutants and other derivatives of TPL-2 preferably retain substantial homology with TPL-2. As used herein, “homology” means that the two entities share sufficient characteristics for the skilled person to determine that they are similar in origin and function. Preferably, homology is used to refer to sequence identity, and is determined as defined above.

In one embodiment, different forms of a TPL-2 protein include, e.g., various amino acid regions of human TPL-2 homolog termed COT and in particular include, e.g., a human TPL-2 polypeptide representing amino acid residues 30 through 397 (i.e., COT (30-397)), a human TPL-2 polypeptide representing amino acid residues 30 through 467 (i.e., COT (30-467)), a human TPL-2 polypeptide representing amino acid residues 1 through 397 (i.e., COT(1-397)) and a human TPL-2 polypeptide representing amino acid residues 1 through 467 (i.e., COT(1-467)). These different forms of TPL-2 polypeptide may be fused to various immuno- or affinity tags known in the art to aid in purification of a given polypeptide. Tags include, but are not restricted to, FLAG tag, GST (glutathione-S-transferase), and poly-histidine residues, e.g., His₆. In addition, the invention also encompasses polypeptides engineered to have, e.g., desirable protease cleavage sites that can be inserted adjacent to the above-mentioned tags to facilitate their removal after protein purification.

Accordingly, the TPL-2 polypeptides of the invention may be expressed and purified by immunoprecipitation from e.g., transfected human 293A cells or from, e.g., baculovirus-infected insect cells as described herein. Typically, baculovirus infected insect cells allow for the purification of large amounts of recombinantly expressed protein suitable for mass-screening of chemical libraries. Further methods for the preparation of a TPL-2 molecule are described below.

1c. Preparation of a TPL-2 Molecule

The invention encompasses the production of TPL-2 molecules for use in the modulation of p105 activity as described above. Preferably, TPL-2 molecules are produced by recombinant DNA technology, by means of which a nucleic acid encoding a TPL-2 molecule can be incorporated into a vector for further manipulation. As used herein, vector (or plasmid) refers to discrete elements that are used to introduce heterologous DNA into cells for either expression or replication thereof. Selection and use of such vehicles are well within the skill of the artisan. Many vectors are available, and selection of appropriate vector will depend on the intended use of the vector, i.e. whether it is to be used for DNA amplification or for DNA expression, the size of the DNA to be inserted into the vector, and the host cell to be transformed with the vector. Each vector contains various components depending on its function (amplification of DNA or expression of DNA) and the host cell for which it is compatible. The vector components generally include, but are not limited to, one or more of the following: an origin of replication, one or more marker genes, an enhancer element, a promoter, a transcription termination sequence and a signal sequence.

Both expression and cloning vectors generally contain nucleic acid sequence that enable the vector to replicate in one or more selected host cells. Typically in cloning vectors, this sequence is one that enables the vector to replicate independently of the host chromosomal DNA, and includes origins of replication or autonomously replicating sequences. Such sequences are well known for a variety of bacteria, yeast and viruses. The origin of replication from the plasmid pBR322 is suitable for most Gram-negative bacteria, the 2p plasmid origin is suitable for yeast, and various viral origins (e.g. SV 40, polyoma, adenovirus) are useful for cloning vectors in mammalian cells. Generally, the origin of replication component is not needed for mammalian expression vectors unless these are used in mammalian cells competent for high level DNA replication, such as COS cells.

Most expression vectors are shuttle vectors, i.e. they are capable of replication in at least one class of organisms but can be transfected into another class of organisms for expression. For example, a vector is cloned in E. coli and then the same vector is transfected into yeast or mammlalian cells even though it is not capable of replicating independently of the host cell chromosome. DNA may also be replicated by insertion into the host genome. However, the recovery of genomic DNA encoding TPL-2 is more complex than that of exogenously replicated vector because restriction enzyme digestion is required to excise TPL-2 DNA. DNA can be amplified by PCR and be directly. transfected into the host cells without any replication component.

Advantageously, an expression and cloning vector may contain a selection gene also referred to as selectable marker. This gene encodes a protein necessary for the survival or growth of transformed host cells grown in a selective culture medium. Host cells not transformed with the vector containing the selection gene will not survive in the culture medium. Typical selection genes encode proteins that confer resistance to antibiotics and other toxins, e.g. ampicillin, neomycin, methotrexate or tetracycline, complement auxotrophic deficiencies, or supply critical nutrients not available from complex media.

As to a selective gene marker appropriate for yeast, any marker gene can be used which facilitates the selection for transformants due to the phenotypic expression of the marker gene. Suitable markers for yeast are, for example, those conferring resistance to antibiotics G418, hygromycin or bleomycin, or provide for prototrophy in an auxotrophic yeast mutant, for example the URA3, LEU2, LYS2, TRP1, or HIS3 gene.

Since the replication of vectors is conveniently done in E. coli, an E. coli genetic marker and an E. coli origin of replication are advantageously included. These can be obtained from E. coli plasmids, such as pBR322, Bluescript© vector or a pUC plasmid, e.g. pUC18 or pUC19, which contain both E. coli replication origin and E. coli genetic marker conferring resistance to antibiotics, such as ampicillin.

Suitable selectable markers for mammalian cells are those that enable the identification of cells competent to take up TPL-2 nucleic acid, such as dihydrofolate reductase (DHFR, methotrexate resistance), thymidine kinase, or genes conferring resistance to G418 or hygromycin. The mammalian cell transformants are placed under selection pressure which only those transformants which have taken up and are expressing the marker are uniquely adapted to survive. In the case of a DHFR or glutamine synthase (GS) marker, selection pressure can be imposed by culturing the transformants under conditions in which the pressure is progressively increased, thereby leading to amplification (at its chromosomal integration site) of both the selection gene and the linked DNA that encodes TPL-2. Amplification is the process by which genes in greater demand for the production of a protein critical for growth, together with closely associated genes which may encode a desired protein, are reiterated in tandem within the chromosomes of recombinant cells. Increased quantities of desired protein are usually synthesized from thus amplified DNA.

Expression and cloning vectors usually contain a promoter that is recognized by the host organism and is operably linked to TPL-2 nucleic acid. Such a promoter may be inducible or constitutive. The promoters are operably linked to DNA encoding TPL-2 by removing the promoter from the source DNA by restriction enzyme digestion and inserting the isolated promoter sequence into the vector. Both the native TPL-2 promoter sequence and many heterologous promoters may be used to direct amplification and/or expression of TPL-2 DNA. The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences.

Promoters suitable for use with prokaryotic hosts include, for example, the, β-lactamase and lactose promoter systems, alkaline phosphatase, the tryptophan (trp) promoter system and hybrid promoters such as the tac promoter. Their nucleotide sequences have been published, thereby enabling the skilled worker operably to ligate them to DNA encoding TPL-2, using linkers or adaptors to supply any required restriction sites. Promoters for use in bacterial systems will also generally contain a Shine-Delgarno sequence operably linked to the DNA encoding TPL-2.

Preferred expression vectors are bacterial expression vectors which comprise a promoter of a bacteriophage such as phagex or T7 which is capable of functioning in the bacteria. In one of the most widely used expression systems, the nucleic acid encoding the fusion protein may be transcribed from the vector by T7 RNA polymerase (Studier et al, Methods in Enzymol. 185; 60-89, 1990). In the E. coli BL21(DE3) host strain, used in conjunction with pET vectors, the T7 RNA polymerase is produced from the \-lysogen DE3 in the host bacterium, and its expression is under the control of the IPTG inducible lac UV5 promoter. This system has been employed successfully for over-production of many proteins. Alternatively the polymerase gene may be introduced on a lambda phage by infection with an int-phage such as the CE6 phage which is commercially available (Novagen, Madison, USA). other vectors include vectors containing the lambda PL promoter such as PLEX (Invitrogen, NL), vectors containing the trc promoters such as pTrcHisXpressTm (Invitrogen) or pTrc99 (Pharmacia Biotech, SE), or vectors containing the tac promoter such as pKK223-3 (Pharmacia Biotech) or PMAL (new England Biolabs, Mass., USA).

Moreover, the TPL-2 gene according to the invention preferably includes a secretion sequence in order to facilitate secretion of the polypeptide from bacterial hosts, such that it will be produced as a soluble native peptide rather than in an inclusion body. The peptide may be recovered from the bacterial perntplasmic space, or the culture medium, as appropriate.

Suitable promoting sequences for use with yeast hosts may be regulated or constitutive and are preferably derived from a highly expressed yeast gene, especially a Saccharomyces cerevisiae gene. Thus, the promoter of the TRP1 gene, the ADHI or ADHII gene, the acid phosphatase (PH05) gene, a promoter of the yeast mating pheromone genes coding for the a- or a-factor or a promoter derived from a gene encoding a glycolytic enzyme such as the promoter of the enolase, glyceraldeLyde-3-phosphate dehydrogenase (GAP), 3-phospho glycerate kinase (PGK), hexokinase, pyruvate decarboxylase, phosphofructokinase, glucose-6-phosphate isomerase, 3-phosphoglycerate mutase, pyruvate kinase, triose phosphate isomerase, phosphoglucose isomerase or glucokinase genes, the S. cerevisiae GAL 4 gene, the S. pombe nmt 1 gene or a promoter from the TATA binding protein (TBP) gene can be used. Furthermore, it is possible to use hybrid promoters comprising upstream activation sequences (UAS) of one yeast gene and downstream promoter elements including a functional TATA box of another yeast gene, for example a hybrid promoter including the UAS(s) of the yeast PH05 gene and downstream promoter elements including a functional TATA box of the yeast GAP gene (PH05-GAP hybrid promoter). A suitable constitutive PH05 promoter is e.g. a shortened acid phosphatase PH05 promoter devoid of the upstream regulatory elements (UAS) such as the PH05 (−173) promoter element starting at nucleotide −173 and ending at nucleotide −9 of the PH05 gene.

TPL-2 gene transcription from vectors in mammalian hosts may be controlled by promoters derived from the genomes of viruses such as polyoma virus, adenovirus, fowlpox virus, bovine papilloma virus, avian sarcoma virus, cytomegalovirus (CMV), a retrovirus and Simian Virus 40 (SV40), from heterologous mammalian promoters such as the actin promoter or a very strong promoter, e.g. a ribosomal protein promoter, and from the promoter normally associated with TPL-2 sequence, provided such promoters are compatible with the host cell systems.

Transcription of a DNA encoding TPL-2 by higher eukaryotes may be increased by inserting an enhancer sequence into the vector. Enhancers are relatively orientation and position independent. Many enhancer sequences are known from mammalian genes (e.g. elastase and globin). However, typically one will employ an enhancer from a eukaryotic cell virus. Examples include the SV40 enhancer on the late side of the replication origin (bp 100-270) and the CMV early promoter enhancer. The enhancer may be spliced into the vector at a position 5′ or 3′ to TPL-2 DNA, but is preferably located at a site 5′ from the promoter.

Advantageously, a eukaryotic expression vector encoding TPL-2 may comprise a locus control region (LCR). LCRs are capable of directing high-level integration site independent expression of transgenes integrated into host cell chromatin, which is of importance especially where the TPL-2 gene is to be expressed in the context of a perranently-transfected eukaryotic cell line in which chromosomal integration of the vector has occurred, in vectors designed for gene therapy applications or in transgenic animals.

Eukaryotic expression vectors will also contain sequences necessary for the termination of transcription and for stabilizing the mRNA. Such sequences are commonly available from the 5′ and 3′ untranslated regions of eukaryotic or viral DNAs or cDNAs. These regions contain nucleotide segments transcribed as polyadenylated fragments in the untranslated portion of the mRNA encoding TPL-2.

An expression vector includes any vector capable of expressing TPL-2 nucleic acids that are operatively linked with regulatory sequences, such as promoter regions, that are capable of expression of such DNAs. Thus, an expression vector refers to a recombinant DNA or RNA construct, such as a plasmid, a phage, recombinant virus or other vector, that upon introduction into an appropriate host cell, results in expression of the cloned DNA. Appropriate expression vectors are well known to those with ordinary skill in the art and include those that are replicable in eukaryotic and/or prokaryotic cells and those that remain episomal or those which integrate into the host cell genome. For example, DNAs encoding TPL-2 may be inserted into a vector suitable for expression of cDNAs in mammalian cells, e.g. a CMV enhancer-based vector such as pEVRF (Matthias, et al., (1989) NAR 17, 6418).

Particularly useful for practicing the present invention are expression vectors that provide for the transient expression of DNA encoding TPL-2 in mammalian cells. Transient expression usually involves the use of an expression vector that is able to replicate efficiently in a host cell, such that the host cell accumulates many copies of the expression vector, and, in turn, synthesizes high levels of TPL-2. For the purposes of the present invention, transient expression systems are useful e.g. for identifying TPL-2 mutants, to identify potential phosphorylation sites, or to characterize functional domains of the protein.

Construction of vectors according to the invention employs conventional ligation techniques. Isolated plasmids or DNA fragments are cleaved, tailored, and religated in the form desired to generate the plasmids required. If desired, analysis to confirm correct sequences in the constructed plasmids is performed in a known fashion. Suitable methods for constructing expression vectors, preparing in vitro transcripts, introducing DNA into host cells, and performing analyses for assessing TPL-2 expression and function are known to those skilled in the art. Gene presence, amplification and/or expression may be measured in a sample directly, for example, by conventional Southern blotting, Northern blotting to quantitate the transcription of mRNA, dot blotting (DNA or RNA analysis), or in situ hybridization, using an appropriately labeled probe which may be based on a sequence provided herein. Those skilled in the art will readily envisage how these methods may be modified, if desired.

Thus, the invention comprises host cells transformed with vectors encoding a heterologous TPL-2 molecule. As used herein, a heterologous TPL-2 molecule may be a mutated form of the endogenous TPL-2, or a mutated or wild-type form of an exogenous TPL-2.

TPL-2 may advantageously be expressed in insect cell systems. Insect cells suitable for use in the method of the invention include, in principle, any lepidopteran cell which is capable of being transformed with an expression vector and expressing heterologous proteins encoded thereby. In particular, use of the Sf cell lines, such as the Spodoptera frugiperda cell line IPBL-SF-21 AE (Vaughn et al., (1977) In vitro, 13, 213-217) is preferred. The derivative cell line Sf9 is particularly preferred. However, other cell lines, such as Tricoplusia ni 368 (Kurstack and Marmorosch, (1976) Invertebrate Tissue Culture Applications in Medicine, Biology and Agriculture. Academic Press, New York, USA) may be employed. These cell lines, as well as other insect cell lines suitable for use in the invention, are commercially available (e.g. from Stratagene, La Jolla, Calif., USA).

As well as expression in insect cells in culture, the invention also comprises the expression of TPL-2 proteins in whole insect organisms. The use of virus vectors such as baculovirus allows infection of entire insects, which are in some ways easier to grow than cultured cells as they have fewer requirements for special growth conditions. Large insects, such as silk moths, provide a high yield of heterologous protein. The protein can be extracted from the insects according to conventional extraction techniques.

Expression vectors suitable for use in the invention include all vectors which are capable of expressing foreign proteins in insect cell lines. In general, vectors which are useful in mammalian and other eukaryotic cells are also applicable to insect cell culture. Baculovirus vectors, specifically intended for insect cell culture, are especially preferred and are widely obtainable commercially (e.g. from Invitrogen and Clontech). Other virus vectors capable of infecting insect cells are known, such as Sindbis virus (Hahn et al., (1992) PNAS (USA) 89, 2679-2683). The baculovirus vector of choice (reviewed by Miller (1988) Ann. Rev. Microbiol. 42, 177-199) is Autographa californica multiple nuclear polyhedrosis virus, AcMNPV.

Typically, the heterologous gene replaces at least in part the polyhedrin gene of AcMNPV, since polyhedrin is not required for virus production. In order to insert the heterologous gene, a transfer vector is advantageously used. Transfer vectors are prepared in E. coli hosts and the DNA insert is then transferred to AcMNPV by a process of homologous recombination.

2. TPL-2 Interaction with p105

The present invention demonstrates that TPL-2 stability in vivo exhibits its high affinity, stoichiometric association with NF-κB1 p105 via two distinct interactions. The TPL-2 C-terminus binds to a region N-terrrinal to the p105 ankyrin repeats (residues 498-541), while the TPL-2 kinase domain binds to the p105 “death domain” (p105 DD). Surprisingly, the MEK kinase activity of TPL2 was found to be profoundly inhibited by its interaction with the p105 DD. The simultaneous interaction of the C-terminus of TPL-2 with residues 498-541 of p105 significantly increased the inhibitory effect of the p105 DD (by increasing the overall affinity of interaction between TPL-2 and p105). These data demonstrate that p105 functions upstream of TPL2 as a negative regulator.

Our invention reveals a mechanism by which deletion of the C-terminus could lead to aberrant TPL-2 activity. Although TPL-2ΔC binds to p105 when expressed in cells, its kinase activity is largely unaffected by this association, in contrast to the full length protein which is dramatically inhibited. The C-termninus of TPL-2 is therefore useful for negative regulation of its kinase activity by the p105 DD and its removal generates an oncogenic form that has much higher specific activity than wild type in cells which co-express p105. Persistent phosphorylation of MEK by TPL-2ΔC is useful for TPL-2□C to transform cells, as constitutively active MEK mutants will oncogenically transform tissue culture cells (Cowley, S., et al. (1994) Cell 77:841-852.7).

In the present invention, in which both kinases were isolated in an effectively p105-free form from lysates of transfected 293 cells, Myc-TPL-2ΔC was found to have higher MEK kinase activity than Myc-TPL-2 (2-3 fold). GST fusion protein encoding the C-terminus of TPL-2 binds to TPL-2ΔC when the two proteins are expressed in baculovirus-infected Sf9 cells and inhibits TPL-2ΔC kinase activity in vitro. Surprisingly, although TPL-2₃₉₈₋₄₆₇ peptide bound to TPL-2ΔC, it was not found to affect its in vitro MEK kinase activity. It is apparent from our invention that the physiologically relevant forms of TPL-2 and TPL-2ΔC exist in a complex with p105. Thus, p105 is an obligate partner for TPL-2 in vivo, that is required to maintain its metabolic stability.

The present invention reveals a novel mechanism by which LPS stimulation triggers phosphorylation of MEK 1 and 2 in macrophages via TPL-2 . Since, essentially all of TPL-2 is complexed with p105 in macrophages, TPL-2 is subject to p105 negative regulation. Presumably, LPS stimulation activates TPL-2 to induce MEK phosphorylation. This occurs as a consequence of LPS stimulation inducing proteolysis of p105 (Delhase, M., et al. (1999) Science 284: 309-313), thereby releasing TPL-2 from its inhibitor.

In addition, Kinetic experiments with bone marrow-derived macrophages demonstrate that LPS induction of MEK phosphorylation precedes p105 proteolysis. Indeed, LPS stimulation actually induces proteolysis of TPL-2 itself by the proteasome, which occurs coincidentally with p105 proteolysis. TPL-2 proteolysis after LPS stimulation therefore constitutes a down-regulatory mechanism. The present invention demonstrates that LPS induces the disruption of one or both of the interactions between TPL-2 and p105, thus relieving p105 negative regulation occurring as a consequence of either TPL-2 or p105 phosphorylation.

3. TPL-2 is a Drug Development Target

According to the present invention, a TPL-2 molecule is used as a target to identify compounds, for example lead compounds for pharmaceuticals, which are capable of modulating the activity of NFκB via p105 proteolysis and Rel subunit release. Accordingly, the invention relates to an assay and provides a method for identifying a compound or compounds capable, directly or indirectly, of modulating the activity of p105, comprising the steps of:

(a) incubating a TPL-2 molecule with the compound or compounds to be assessed; and

(b) identifying those compounds which influence the activity of the 1 PL-2 molecule.

3a. TPL-2 Binding Compounds

According to a first embodiment of this aspect invention, the assay is configured to detect polypeptides which bind directly to the TPL-2 molecule.

The invention therefore provides a method for identifying a modulator of NFκB activity, comprising the steps of:

(a) incubating a TPL-2 molecule with the compound or compounds to be assessed; and

(b) identifying those compounds which bind to the TPL-2 molecule. Preferably, the method further comprises the step of:

(c) assessing the compounds which bind to TPL-2 for the ability to modulate NFκB activation in a cell-based assay.

Binding to TPL-2 may be assessed by any technique known to those skilled in the art. Examples of suitable assays include the two hybrid assay system, which measures interactions in vivo, affinity chromatography assays, for example involving binding to polypeptides immobilized on a column, fluorescence assays in which binding of the compound(s) and TPL-2 is associated with a change in fluorescence of one or both partners in a binding pair, and the like. Preferred are assays performed in vivo in cells, such as the two-hybrid assay.

In a preferred aspect of this embodiment, the invention provides a method for identifying a lead compound for a pharmaceutical useful in the treatment of disease involving or using an inflammatory response, comprising incubating a compound or compounds to be tested with a TPL-2 molecule and p105, under conditions in which, but for the presence of the compound or compounds to be tested, TPL-2 associates with p105 with a reference affinity;

determining the binding affinity of TPL-2 for p105 in the presence of the compound or compounds to be tested; and

selecting those compounds which modulate the binding affinity of TPL-2 for p105 with respect to the reference binding affinity.

Preferably, therefore, the assay according to the invention is calibrated in absence of the compound or compounds to be tested, or in the presence of a reference compound whose activity in binding to TPL-2 is known or is otherwise desirable as a reference value. For example, in a two-hybrid system, a reference value may be obtained in the absence of any compound. Addition of a compound or compounds which increase the binding affinity of TPL-2 for p105 increases the readout from the assay above the reference level, whilst addition of a compound or compounds which decrease this affinity results in a decrease of the assay readout below the reference level.

3b. Compounds which Modulate the Functional p105/TPL-2 Interaction

In a second embodiment, the invention may be configured to detect functional interactions between a compound or compounds and TPL-2. Such interactions will occur either at the level of the regulation of TPL-2, such that this kinase is itself activated or inactivated in response to the compound or compounds to be tested, or at the level of the modulation of the biological effect of TPL-2 on p105. As used herein, “activation”, and “inactivation” include modulation of the activity, enzymatic or otherwise, of a compound, as well as the modulation of the rate of production thereof, for example by the activation or repression of expression of a polypeptide in a cell. The terms include direct action on gene transcription in order to modulate the expression of a gene product.

Assays which detect modulation of the functional interaction between TPL-2 and p105 are preferably cell-based assays. For example, they may be based on an assessment of the degree of phosphorylation of p105, which is indicative of the degree of NFκB activation, resulting from the TPL-2-p105 interaction.

In preferred embodiments, a nucleic acid encoding a TPL-2 molecule is ligated into a vector, and introduced into suitable host cells to produce transformed cell lines that express the TPL-2 molecule. The resulting cell lines can then be produced for reproducible qualitative and/or quantitative analysis of the effect(s) of potential compounds affecting TPL-2 function. Thus TPL-2 expressing cells may be employed for the identification of compounds, particularly low molecular weight compounds, which modulate the function of TPL-2. Thus host cells expressing TPL-2 are useful for drug screening and it is a further object of the present invention to provide a method for identifying compounds which modulate the activity of TPL-2, said method comprising exposing cells containing heterologous DNA encoding TPL-2, wherein said cells produce functional TPL-2, to at least one compound or mixture of compounds or signal whose ability to modulate the activity of said TPL-2 is sought to be determined, and thereafter monitoring said cells for changes caused by said modulation. Such an assay enables the identification of modulators, such as agonists, antagonists and allosteric modulators, of TPL-2. As used herein, a compound or signal that modulates the activity of TPL-2 refers to a compound that alters the activity of TPL-2 in such a way that the activity of TPL-2 in p105 activation is different in the presence of the compound or signal (as compared to the absence of said compound or signal).

Cell-based screening assays can be designed by constructing cell lines in which the expression of a reporter protein, i.e. an easily assayable protein, such as β-galactosidase, chloramphenicol acetyltransferase (CAT) or luciferase, is dependent on 34 the activation of p105 by TPL-2. For example, a reporter gene encoding one of the above polypeptides may be placed under the control of an NFκB-response element which is specifically activated p50. Where the element is activated by p50 heterodimers, provision must be made for expression of alternative Ret monomers at a predictable level. Such an assay enables the detection of compounds that directly modulate TPL-2 function, such as compounds that antagonize phosphorylation of p105 by TPL-2, or compounds that inhibit or potentiate other cellular functions required for the activity of TPL-2.

Alternative assay formats include assays which directly assess inflammatory responses in a biological system. It is known that constitutive expression of unregulated p50 results in an inflammatory phenotype in animals. Cell-based systems, such as those dependent on cytokine release or cell proliferation, may be used to assess the activity of p50.

In a preferred aspect of this embodiment of the invention, there is provided a method for identifying a lead compound for a pharmaceutical useful in the treatment of disease involving or using an inflammatory response, comprising:

incubating a compound or compounds to be tested with a TPL-2 molecule and p105, under conditions in which, but for the presence of the compound or compounds to be tested, TPL-2 directly or indirectly causes the phosphorylation of p105 with a reference phosphorylation efficiency;

determining the ability of TPL-2 to cause the phosphorylation, directly or indirectly, of p105 in the presence of the compound or compounds to be tested; and selecting those compounds which modulate the ability of TPL-2 to phosphorylate p105 with respect to the reference phosphorylation efficiency.

In the case where TPL-2 indirectly phosphorylates a target polypeptide, e.g., p105, a further kinase or kinases may be involved and thus, the assays according to the present embodiment of the invention may be advantageously configured to detect indirect target polypeptide or p105 phosphorylation by TPL-2.

In a further preferred aspect, the invention relates to a method for identifying a lead compound for a pharmaceutical, comprising the steps of:

providing a purified TPL-2 molecule;

incubating the TPL-2 molecule with a substrate known to be phosphorylated by TPL-2 and a test compound or compounds; and

identifying the test compound or compounds capable of modulating the phosphorylation of the substrate.

A substrate for TPL-2 phosphorylation is MEK(EMBOJ.15:817-826,1996). Preferably, therefore, MEK is used as a substrate to monitor compounds capable of modulating TPL2 kinase activity. In another embodiment, the test substrate may be any suitable TPL-2 target polypeptide, such as, e.g., MEK-1, SEK-1, IκB-α, IκB-β, NF-κB1 p105, NFκB and TPL-2/COT itself. In particular, the invention provides recombinant, fusion protein constructs for making these substrates e.g., as convenient model fusion proteins. In a preferred embodiment, model fusion proteins include, e.g., GST-IκB-α (1-50), i.e., amino acid residues 1 through 50 of IκB-α fused to GST, and GST-p105Ndel.498 (comprising residues 498-969 of p105). Other peptide substrates for TPL-2/COT may be derived from these protein substrates, and include for example the IκB-α-derived peptide NH₂-DDRHDSGLDSMKDKKK-COOH (where the serine residue in bold corresponds to serine residue 32 of IκB-α) and the MEK-derived peptide NH₂-QLIDSMANSFVGTKKK-COOH (where the serine residue in bold corresponds to serine residue 217 of MEK-1). These and other TPL-2 target polypeptides described herein allows for a person skilled in the art to screen directly for kinase modulators. Preferably, kinase modulators are kinase (TPL-2) inhibitors.

Optionally, the test compound(s) identified may then be subjected to in vivo testing to determine their effects on a TNF/p105 originating signaling pathway, for example as set forth in the foregoing embodiment.

3c. Compounds which Modulate TPL-2 Activity.

As used herein, “TPL-2 activity” may refer to any activity of TPL-2, including its binding activity, but in particular refers to the phosphorylating activity of TPL-2.

Accordingly, the invention may be configured to detect the phosphorylation of target compounds by TPL-2, and the modulation of this activity by potential therapeutic agents.

Examples of compounds which modulate the phosphorylating activity of TPL-2 include dominant negative mutants of TPL-2 itself. Such compounds are able to compete for the target of TPL-2, thus reducing the activity of TPL-2 in a biological or artificial system. Thus, the invention moreover relates to compounds capable of modulating the phosphorylating activity of TPL-2.

4. Compounds

In a still further aspect, the invention relates to a compound or compounds identifiable by an assay method as defined in the previous aspect of the invention. Accordingly, there is provided the use of a compound identifiable by an assay as described herein, for the modulation of the activity of NFκB.

Compounds which influence the TPL-2/NFκB interaction may be of almost any general description, including low molecular weight compounds, including organic compounds which may be linear, cyclic, polycyclic or a combination thereof, peptides, polypeptides including antibodies, or proteins. In general, as used herein, “peptides”, “polypeptides” and “proteins” are considered equivalent.

4a. Antibodies

Antibodies, as used herein, refers to complete antibodies or antibody fragments capable of binding to a selected target, and including Fv, ScFv, Fab′ and F(ab′)2, monoclonal and polyclonal antibodies, engineered antibodies including chimeric, CDR-grafted and humanized antibodies, and artificially selected antibodies produced using phage display or alternative techniques. Small fragments, such Fv and ScFv, possess advantageous properties for diagnostic and therapeutic applications on account of their small size and consequent superior tissue distribution.

The antibodies according to the invention are especially indicated for diagnostic and therapeutic applications. Accordingly, they may be altered antibodies comprising an effector protein such as a toxin or a label. Especially preferred are labels which allow the imaging of the distribution of the antibody in vivo. Such labels may be radioactive labels or radioopaque labels, such as metal particles, which are readily visualizable within the body of a patient. Moreover, the may be fluorescent labels or other labels which are visualizable on tissue samples removed from patients.

Recombinant DNA technology may be used to improve the antibodies of the invention. Thus, chimeric antibodies may be constructed in order to decrease the immunogenicity thereof in diagnostic or therapeutic applications. Moreover, immunogenicity may be minimized by humanizing the antibodies by CDR grafting [see European Patent Application 0 239 400 (Winter)] and, optionally, framework modification [see international patent application WO 90/07861 (Protein Design Labs)].

Antibodies according to the invention may be obtained from animal serum, or, in the case of monoclonal antibodies or fragments thereof, produced in cell culture. Recombinant DNA technology may be used to produce the antibodies according to established procedure, in bacterial or preferably mammalian cell culture. The selected cell culture system preferably secretes the antibody product.

Therefore, the present invention includes a process for the production of an antibody according to the invention comprising culturing a host, e.g. E. coli or a mammalian cell, which has been transformed with a hybrid vector comprising an expression cassette comprising a promoter operably linked to a first DNA sequence encoding a signal peptide linked in the proper reading frame to a second DNA sequence encoding said protein, and isolating said protein.

Multiplication of hybridoma cells or mammalian host cells in vitro is carried out in suitable culture media, which are the customary standard culture media, for example Dulbecco's Modified Eagle Medium (DMEM) or RPMI 1640 medium, optionally replenished by a mammalian serum, e.g. fetal calf serum, or trace elements and growth sustaining supplements, e.g. feeder cells such as normal mouse peritoneal exudate cells, spleen cells, bone marrow macrophages, 2-aminoethanol, insulin, transferrin, low density lipoprotein, oleic acid, or the like. Multiplication of host cells which are bacterial cells or yeast cells is likewise carried out in suitable culture media known in the art, for example for bacteria in medium LB, NZCYM, NZYM, NZM, Terrific Broth, SOB, SOC, 2×YT, or M9 Minimal Medium, and for yeast in medium YPD, YEPD, Minimal Medium, or Complete Minimal Dropout Medium.

In vitro production provides relatively pure antibody preparations and allows scale-up to give large amounts of the desired antibodies. Techniques for bacterial cell, yeast or mammalian cell cultivation are known in the art and include homogeneous suspension culture, e.g. in an airlift reactor or in a continuous stirrer reactor, or immobilized or entrapped cell culture, e.g. in hollow fibers, microcapsules, on agarose microbeads or ceramic cartridges.

Large quantities of the desired antibodies can also be obtained by multiplying mammalian cells in vivo. For this purpose, hybridoma cells producing the desired antibodies are injected into histocompatible mammals to cause growth of antibody-producing tumors. Optionally, the animals are primed with a hydrocarbon, especially mineral oils such as pristane (tetramethyl-pentadecane), prior to the injection. After one to three weeks, the antibodies are isolated from the body fluids of those mammals. For example, hybridoma cells obtained by fusion of suitable myeloma cells with antibody-producing spleen cells from Balb/c mice, or transfected cells derived from hybridoma cell line Sp2/0 that produce the desired antibodies are injected intraperitoneally into Balb/c mice optionally pre-treated with pristane, and, after one to two weeks, ascitic fluid is taken from the animals.

The foregoing, and other, techniques are discussed in, for example, Kohler and Milstein, (1975) Nature 256:495-497; U.S. Pat. No. 4,376,110; Harlow and Lane, Antibodies: a Laboratory Manual, (1988) Cold Spring Harbor, incorporated herein by reference. Techniques for the preparation of recombinant antibody molecules is described in the above references and also in, for example, EP 0623679; EP 0368684 and EP 0436597, which are incorporated herein by reference.

The cell culture supernatants are screened for the desired antibodies, preferentially by immunofluorescent staining of cells expressing TPL-2 by immunoblotting, by an enzyme immunoassay, e.g. a sandwich assay or a dot-assay, or a radioimmunoassay.

For isolation of the antibodies, the immunoglobulins in the culture supernatants or in the ascitic fluid may be concentrated, e.g. by precipitation with ammonium sulphate, dialysis against hygroscopic material such as polyethylene glycol, filtration through selective membranes, or the like. If necessary and/or desired, the antibodies are purified by the customary chromatography methods, for example gel filtration, ion-exchange chromatography, chromatography over DEAE-cellulose and/or (immuno-) affinity chromatography, e.g. affinity chromatography with a TPL-2 molecule or with Protein-A. The invention further concerns hybridoma cells secreting the monoclonal antibodies of the invention. The preferred hybridoma cells of the invention are genetically stable, secrete monoclonal antibodies of the invention of the desired specificity and can be activated from deep-frozen cultures by thawing and recloning.

The invention also concerns a process for the preparation of a hybridoma cell line secreting monoclonal antibodies directed to a TPL-2 molecule, characterised in that a suitable mammal, for example a Balb/c mouse, is immunized with a purified TPL-2 molecule, an antigenic carrier containing a purified TPL-2 molecule or with cells bearing TPL-2, antibody-producing cells of the immunized mammal are fused with cells of a suitable myeloma cell line, the hybrid cells obtained in the fusion are cloned, and cell clones secreting the desired antibodies are selected. For example spleen cells of Balb/c mice immunized with cells bearing TPL-2 are fused with cells of the myeloma cell line PAI or the myeloma cell line Sp2/0-Ag14, the obtained hybrid cells are screened for secretion of the desired antibodies, and positive hybridoma cells are cloned.

Preferred is a process for the preparation of a hybridoma cell line, characterized in that Balb/c mice are immunized by injecting subcutaneously and/or intraperitoneally between 10⁷ and 10⁸ cells of human tumour origin which express TPL-2 containing a suitable adjuvant several times, e.g. four to six times, over several months, e.g. between two and four months, and spleen cells from the immunized mice 40 are taken two to four days after the last injection and fused with cells of the myeloma cell line PAI in the presence of a fusion promoter, preferably polyethylene glycol. Preferably the myeloma cells are fused with a three- to twentyfold excess of spleen cells from the immunized mice in a solution containing about 30% to about 50% polyethylene glycol of a molecular weight around 4000. After the fusion the cells are expanded in suitable culture media as described hereinbefore, supplemented with a selection medium, for example HAT medium, at regular intervals in order to prevent normal myeloma cells from overgrowing the desired hybridoma cells.

The invention also concerns recombinant DNAs comprising an insert coding for a heavy chain variable domain and/or for a light chain variable domain of antibodies directed to a TPL-2 molecule as described hereinbefore. By definition such DNAs comprise coding single stranded DNAs, double stranded DNAs consisting of said coding DNAs and of complementary DNAs thereto, or these complementary (single stranded) DNAs themselves.

Furthermore, DNA encoding a heavy chain variable domain and/or for a light chain variable domain of antibodies directed to a TPL-2 molecule can be enzymatically or chemically synthesized DNA having the authentic DNA sequence coding for a heavy chain variable domain and/or for the light chain variable domain, or a mutant thereof. A mutant of the authentic DNA is a DNA encoding a heavy chain variable domain and/or a light chain variable domain of the above-mentioned antibodies in which one or more amino acids are deleted or exchanged with one or more other amino acids. Preferably said modification(s) are outside the CDRs of the heavy chain variable domain and/or of the light chain variable domain of the antibody. Such a mutant DNA is also intended to be a silent mutant wherein one or more nucleotides are replaced by other nucleotides with the new codons coding for the same amino acid(s). Such a mutant sequence is also a degenerated sequence. Degenerated sequences are degenerated within the meaning of the genetic code in that an unlimited number of nucleotides are replaced by other nucleotides without resulting in a change of the amino acid sequence originally encoded. Such degenerated sequences may be useful due to their different restriction sites and/or frequency of particular codons which are preferred by the specific host, particularly E. coli, to obtain an optimal expression of the heavy chain murine variable domain and/or a light chain murine variable domain.

The term mutant is intended to include a DNA mutant obtained by in vitro mutagenesis of the authentic DNA according to methods known in the art.

For the assembly of complete tetrameric immunoglobulin molecules and the expression of chimeric antibodies, the recombinant DNA inserts coding for heavy and light chain variable domains are fused with the corresponding DNAs coding for heavy and light chain constant domains, then transferred into appropriate host cells, for example after incorporation into hybrid vectors.

The invention therefore also concerns recombinant DNAs comprising an insert coding for a heavy chain murine variable domain of an antibody directed TPL-2 fused to a human constant domain gamma, for example γ1, γ2, γ3 or y4, preferably γ1 or γ4. Likewise the invention concerns recombinant DNAs comprising an insert coding for a light chain murine variable domain of an antibody directed to TPL-2 fused to a human constant domain κ or λ, preferably κ.

In another embodiment the invention pertains to recombinant DNAs coding for a recombinant polypeptide wherein the heavy chain variable domain and the light chain variable domain are linked by way of a spacer group, optionally comprising a signal sequence facilitating the processing of the antibody in the host cell and/or a DNA coding for a peptide facilitating the purification of the antibody and/or a cleavage site and/or a peptide spacer and/or an effector molecule.

The DNA coding for an effector molecule is intended to be a DNA coding for the effector molecules useful in diagnostic or therapeutic applications. Thus, effector molecules which are toxins or enzymes, especially enzymes capable of catalyzing the activation of prodrugs, are particularly indicated. The DNA encoding such an effector molecule has the sequence of a naturally occurring enzyme or toxin encoding DNA, or a mutant thereof, and can be prepared by methods well known in the art.

Antibodies and antibody fragments according to the invention are useful in diagnosis and therapy. Accordingly, the invention provides a composition for therapy or diagnosis comprising an antibody according to the invention.

In the case of a diagnostic composition, the antibody is preferably provided together with means for detecting the antibody, which may be enzymatic, fluorescent, radioisotopic or other means. The antibody and the detection means may be provided for simultaneous, simultaneous separate or sequential use, in a diagnostic kit intended for diagnosis.

4b. Peptides

Peptides according to the present invention are usefully derived from TPL-2, p105 or another polypeptide involved in the functional TPL-2/p105 interaction. Preferably, the peptides are derived from the domains in TPL-2 or p105 which are responsible for p105/TPL-2 interaction. For example, Thoinberry et al., (1994) Biochemistry 33:39343940 and Milligan et al., (1995) Neuron 15:385-393 describe the use of modified tetrapeptides to inhibit ICE protease. In an analogous fashion, peptides derived from TPL-2, p105 or an interacting protein may be modified, for example with an aldehyde group, chloromethylketone, (acyloxy) methyl ketone or CH2OC(0)-DCB group to inhibit the TPL-2/p105 interaction.

In order to facilitate delivery of peptide compounds to cells, peptides may be modified in order to improve their ability to cross a cell membrane. For example, U.S. Pat. No. 5,149,782 discloses the use of fusogenic peptides, ion-channel forming peptides, membrane peptides, long-chain fatty acids and other membrane blending agents to increase protein transport across the cell membrane. These and other methods are also described in WO 97/37016 and U.S. Pat. No. 5,108,921, incorporated herein by reference.

Many compounds according to the present invention may be lead compounds useful for drug development. Useful lead compounds are especially antibodies and peptides, and particularly intracellular antibodies expressed within the cell in a gene therapy context, which may be used as models for the development of peptide or low molecular weight therapeutics. In a preferred aspect of the invention, lead compounds and TPL-2/p105 or other target peptide may be co-crystallized in order to facilitate the design of suitable low molecular weight compounds which mimic the interaction observed with the lead compound.

Crystallization involves the preparation of a crystallization buffer, for example by mixing a solution of the peptide or peptide complex with a “reservoir buffer”, preferably in a 1:1 ratio, with a lower concentration of the precipitating agent necessary for crystal formation. For crystal formation, the concentration of the precipitating agent is increased, for example by addition of precipitating agent, for example by titration, or by allowing the concentration of precipitating agent to balance by diffusion between the crystallization buffer and a reservoir buffer. Under suitable conditions such diffusion of precipitating agent occurs along the gradient of precipitating agent, for example from the reservoir buffer having a higher concentration of precipitating agent into the crystallization buffer having a lower concentration of precipitating agent. Diffusion may be achieved for example by vapor diffusion techniques allowing diffusion in the common gas phase. Known techniques are, for example, vapor diffusion methods, such as the “hanging drop” or the “sitting drop” method. In the vapor diffusion method a drop of crystallization buffer containing the protein is hanging above or sitting beside a much larger pool of reservoir buffer. Alternatively, the balancing of the precipitating agent can be achieved through a semipermeable membrane that separates the crystallization buffer from the reservoir buffer and prevents dilution of the protein into the reservoir buffer.

In the crystallization buffer the peptide or peptide/binding partner complex preferably has a concentration of up to 30 mg/ml, preferably from about 2 mg/ml to about 4 mg/ml.

Formation of crystals can be achieved under various conditions which are essentially determined by the following parameters: pH, presence of salts and additives, precipitating agent, protein concentration and temperature. The pH may range from about 4.0 to 9.0. The concentration and type of buffer is rather unimportant, and therefore variable, e.g. in dependence with the desired pH. Suitable buffer systems include phosphate, acetate, citrate, Tris, MES and HEPES buffers. Useful salts and additives include e.g. chlorides, sulphates and other salts known to those skilled in the art. The buffer contains a precipitating agent selected from the group consisting of a water miscible organic solvent, preferably polyethylene glycol having a molecular weight of between 100 and 20000, preferentially between 4000 and 10000, or a suitable salt, such as a sulphates, particularly ammonium sulphate, a chloride, a citrate or a tartarate.

A crystal of a peptide or peptide/binding partner complex according to the invention may be chemically modified, e.g. by heavy atom derivatization. Briefly, such derivatization is achievable by soaking a crystal in a solution containing heavy metal atom salts, or a organometallic compounds, e.g. lead chloride, gold thiomalate, thimerosal or uranyl acetate, which is capable of diffusing through the crystal and binding to the surface of the protein. The location(s) of the bound heavy metal atom(s) can be determined by X-ray diffraction analysis of the soaked crystal, which information may be used e.g. to construct a three-dimensional model of the peptide.

A three-dimensional model is obtainable, for example, from a heavy atom derivative of a crystal and/or from all or part of the structural data provided by the crystallization. Preferably building of such model involves homology modeling and/or molecular replacement.

The preliminary homology model can be created by a combination of sequence alignment with any MAPKK kinase or NFκB the structure of which is known (including IκBα, Bauerle et a., (1998) Cell 95:729-731), secondary structure prediction and screening of structural libraries. For example, the sequences of TPL-2 and a candidate peptide can be aligned using a suitable software program.

Computational software may also be used to predict the secondary structure of the peptide or peptide complex. The peptide sequence may be incorporated into the TPL-2 structure. Structural incoherences, e.g. structural fragments around insertions/deletions can be modeled by screening a structural library for peptides of the desired length and with a suitable conformation. For prediction of the side chain conformation, a side chain rotamer library may be employed.

The final homology model is used to solve the crystal structure of the peptide by molecular replacement using suitable computer software. The homology model is positioned according to the results of molecular replacement, and subjected to further refinement comprising molecular dynamics calculations and modeling of the inhibitor used for crystallization into the electron density.

4c. Other Compounds

In a preferred embodiment, the above assay is used to identify peptide but also non-peptide-based test compounds that can modulate TPL-2 activity, e.g., kinase activity, target polypeptide interactions, or signaling activity. The test compounds of the present invention can be obtained using any of the numerous approaches involving combinatorial library methods known in the art, including: biological libraries, spatially addressable parallel solid phase or solution phase libraries; synthetic library methods requiring deconvolution; the ‘one-bead one-compound’ library method; and synthetic library methods using affinity chromatography selection. These approaches are applicable to peptide, non-peptide oligomer, or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner USP 5,223,409), spores (Ladner USP '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

If desired, any of the compound libraries described herein may be divided into pre-selected libraries comprising compounds having, e.g., a given chemical structure, or a given activity, e.g., kinase inhibitory activity. Pre-selecting a compound library may further involve performing any art recognized molecular modeling in order to identify particular compounds or groups or combinations of compounds as likely to have a given activity, reactive site, or other desired chemical functionality. In one embodiment, modulators of TPL-2 are pre-selected using molecular modeling designed to identify compounds having, or likely to have, kinase inhibitory activity.

Suitable methods, as are known in the art, can be used to select particular moieties for interacting with a particular domain of TPL-2 or target component, e.g., p105. For example, visual inspection, particularly utilizing three-dimensional models, can be employed. Preferably, a computer modeling program, or software, is used to select one or more moieties which can interact with a particular domain. Suitable computer modeling programs include QUANTA (Molecular Simulations, Inc., Burlington, Mass. (1992)), SYBYL (Tripos Associates, Inc., St. Louis, Mo. (1992)), AMBER (Weiner et al., J. Am. Chem. Soc. 106: 765-784 (1984)) and CHARMM (Brooks et al., J. Comp. Chem. 4: 187-217 (1983)). Other programs which can be used to select interacting moieties include GRID (Oxford University, U.K.; Goodford et al., J. Mod. Chem. 28: 849-857 (1985)); MCSS (Molecular Simulations, Inc., Burlington, Mass.; Miranker, A. and M. Karplus, Proteins: Structure, Function and Genetics 11: 29-34 (1991)); AUTODOCK (Scripps Research Institute, La Jolla, Calif.; Goodsell et al., Proteins: Structure, Function and Genetics: 195-202 (1990)); and DOCK (University of California, San Francisco, Calif.; Kuntz et al., J. Mol. Biol. 161: 269-288 (1982).

After potential interacting moieties have been selected, they can be attached to a scaffold which can present them in a suitable manner for interaction with the selected domains. Suitable scaffolds and the spatial distribution of interacting moieties thereon can be determined visually, for example, using a physical or computer-generated three-dimensional model, or by using a suitable computer program, such as CAVEAT (University of California, Berkeley, Calif.; Bartlett et al., in “Molecular Recognition of in Chemical and Biological Problems”, Special Pub., Royal Chemical Society 78: 182-196 (1989)); three-dimensional database systems, such as MACCS-3D (MDL Information Systems, San Leandro, Calif. (Martin, Y. C., J. Mod. Chem. 35: 2145-2154 (1992)); and HOOK (Molecular Simulations, Inc.). Other computer programs which can be used in the design and/or evaluation of potential TPL-2 inhibitors include LUDI (Biosym Technologies, San Diego, Calif.; Bohm, H. J., J. Comp. Aid. Molec. Design: 61-78 (1992)), LEGEND (Molecular Simulations, Inc.; Nishibata et al., Tetrahedron 47: 8985-8990 (1991)), and LeapFrog (Tripos Associates, Inc.).

In addition, a variety of techniques for modeling protein-drug interactions are known in the art and can be used in the present method (Cohen et al., J. Med. Chem. 33: 883-894 (1994); Navia et al. Current Opinions in Structural Biology 2: 202-210 (1992); Baldwin et al., J. Mod. Chem. 32: 2510-2513 (1989); Appelt et al.; J. Mod. Chem. 34: 1925-1934 (1991); Ealick et al., Proc. Nat. Acad. Sci. USA 88: 11540-11544 (1991)).

Thus, a library of compounds, e.g., compounds that are protein based, carbohydrate based, lipid based, nucleic acid based, natural organic based, synthetically derived organic based, or antibody based compounds can be assembled and subjected, if desired, to a further preselection step involving any of the aforementioned modeling techniques. Suitable candidate compounds determined to be TPL-2 modulators using these modeling techniques may then be selected from art recognized sources, e.g., commercial sources, or, alternatively, synthesized using art recognized techniques to contain the desired moiety predicted by the molecular modeling to have an activity, e.g., TPL-2 inhibitor activity. These compounds may then be used to form e.g., a smaller or more targeted test library of compounds for screening using the assays described herein.

Accordingly, a desired test library of TPL-2 kinase inhibitors may include, e.g., the compound N-(6-phenoxy-4-quinolyl)-N[4-(phenylsulfanyl)phenyl]amine. The general synthesis of 4-(4-phenylthio-anilino)-quinolinyl derivatives is performed as follows. To a 0.1M solution of ethyl 4-hydroxy-5-oxo-5,6,7,8-tetrahydro-3-quinolinecarboxylate in a 1:1 mixture (v/v) of 1,2-dimethoxyethane and dichloroethane is added carbontetrachloride (10 mol equiv.) and polymer-bound triphenylphosphine (3 to 6 equiv.; Fluka). The mixture is then heated with shaking in a sealed vial at 80° C. for 36 h. 4-(Phenylthio)aniline (2-6 mol equiv.; 0.5M in tert-butanol) is added and the mixture is heated with shaking in a sealed vial to 90° C. for 24 h. Next, the polymer resin is filtered off and washed with methanol. The pooled filtrate and washes are concentrated under high vacuum and the residue was chromatographed by RP-HPLC. By analytical RP-HPLC/MS (0-100% acetonitrile/pH4.5, 50 mM NH₄OAc, at 3.5 mL/min on a Perkin Elmer Pecosphere column (4.6 mm×3 cm)) the ethyl 5-oxo-4-[4-(phenylsulfanyl)anilino]-5,6,7,8-tetrahydro-3-quinolinecarboxylate had a retention time of 3.85 min and MH⁺ at m/z 419.

To prepare N-(6-phenoxy-4-quinolyl)-N-[4-(phenylsulfanyl)phenyl]amine (anal. RP-HPLC RT: 4.32 min.; MS: MH⁺421) in particular, the above procedure is followed using 4-hydroxy-6-phenoxy-quinoline and 4-(phenylthio)aniline.

A related compound, ethyl 5-oxo-4-[4-(phenylsulfanyl)anilino]-5,6,7,8-tetrahydro-3-quinolinecarboxylate, and/or variants thereof, may also be selected for the library and this compound can be produced using standard techniques and the following methodology. Briefly, 10 equivalents of carbontetrachloride and 3-6 equivalents of polymer bound triphenyl phosphine are added to a 0.1 M solution of ethyl 4-hydroxy-5-5,6,7,8-tetrahydro-3-quinolinecarboxylate in a 1:1 mixture of ethylene glycol dimethyl ether and dichloroethane. The mixture is then heated to 80° C. for 36 h. Excess 4-thiophenyaniline (2-6 equivalents) in 250 μl of tert-butanol is added and the mixture is heated to 90° C. for 24 h. The polymer resin is then filtered off, washed with methanol, and remaining solvents are removed under high vacuum to yield the desired test compound.

It is predicted that 4-(4-phenylthio-anilino)-quinolinyl, and derivatives thereof, represent a chemical class that contains compounds suitable for inhibiting a kinase, e.g., a serine/threonine kinase, such as, e.g., COT. Accordingly, any compound comprising the core structure depicted in FIG. 14 is encompassed by the invention. In one embodiment, the quinolinyl ring system may be, e.g., a dihydroquinolinyl or tetrahydroquinolinyl ring system (see FIG. 14, e.g., dotted lines). In addition, the R and R′ groups may be independently selected from: hydroxy, halo, —NHC(O) alkyl, —COOH, —C(O)O-alkyl, —C(O)NH-alkyl, C₁-C₆ alkenyl, C₁-C₆-alkynyl, C₁-C₆-alkyl, C₁-C₆-alkoxy, aryloxy, substituted aryloxy, C₁-C₆-alkylthio, C₁-C₆-alkylamino, cyano, perhalomethyl, perhalomethoxy, amino, mono- or dialkylamino, aryl, substituted aryl, ara-alkyl, and ara-alkoxy. In addition, it is understood that R′ may also represent (R′)n where n=0, 1, 2, etc. such that, e.g., multiple R′ substitutions are allowed. It is also understood that alkyl, alkenyl, and/or alkyonyl groups may be straight or branched chains. Further, it is intended that any salt, or, e.g., where appropriate, analog, free base form, tautomer, enantiomer racemate, or combination thereof, comprising or derived from the generic structure depicted in FIG. 14, is encompassed by the invention.

Another suitable compound for the test library is 3-(4-pyridyl)-4,5-dihydro-2H-benzo[g]indazole methanesulfonate, and/or variants thereof, and this compound is commercially available from Aldrich Chemical Co., Inc. (Registry No. 80997-85-9).

The library may also include the compound sodium 2-chlorobenzo[l][1,9] phenanthroline-7-carboxylate, and/or variants thereof, and this compound can be produced using standard art recognized techniques and using the structure depicted in FIG. 12.

It will be appreciated by one skilled in the art that desired standard modifications of the foregoing compounds may be made using various art recognized techniques and these modified compounds are encompassed by the invention.

In one embodiment, an assay is a cell-based or cell-free assay in which either a cell that expresses, e.g., a TPL-2 polypeptide or cell lysate/or purified protein comprising TPL-2 is contacted with a test compound and the ability of the test compound to alter TPL-2 activity, e.g., kinase activity, target polypeptide interactions, or signaling activity is measured.

Any of the cell-based assays can employ, for example, a cell of eukaryotic or prokaryotic origin. Determining the ability of the test compound to bind to TPL-2 or a TPL-2 target polypeptide can be accomplished, for example, by coupling the test compound with a radioisotope or enzymatic label such that binding of the test compound to the polypeptide can be determined by detecting the labeled compound in a complex. For example, test compounds can be labeled with ¹²⁵I, ³⁵S, ¹⁴C, ³³P, ³²P, or ³H, either directly or indirectly, and the radioisotope detected by direct counting of radioemmission or by scintillation counting. Alternatively, test compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a test compound to interact with a target polypeptide without the labeling of any of the interactants. For example, a microphysiometer can be used to detect the interaction of a test compound with TPL-2 or a target polypeptide without the labeling of either the test compound, TPL-2, or the target polypeptide (McConnell, H. M. et al. (1992) Science 257:1906-1912). In yet another embodiment, an assay of the present invention is a cell-free assay in which, e.g., TPL-2 and a target polypeptide are contacted with a test compound and the ability of the test compound to alter the interaction is deteimiined. This interaction may or may not further include the phosphorylation of TPL-2 and/or the TPL-2 target polypeptide. Binding of the test compound to the target polypeptide can be determined either directly or indirectly. Determining the ability of the candidate compound to bind to either polypeptide can also be accomplished using a technology such as real-time Biomolecular Interaction Analysis (BIA) (Sjolander, S. and Urbaniczky, C. (1991) Anal. Chem. 63:2338-2345 and Szabo et al. (1995) Curr. Opin. Struct. Biol. 5:699-705). As used herein, “BIA” is a technology for studying bispecific interactions in real time, without labeling any of the interactants (e.g., BIAcore™). Changes in the optical phenomenon surface plasmon resonance (SPR) can be used as an indication of real-time reactions between biological molecules.

In many drug screening programs which test libraries of compounds and natural extracts, high throughput assays are desirable in order to maximize the number of compounds surveyed in a given period of time. Assays which are performed in cell-free systems, such as may be performed using purified or semi-purified proteins, are often preferred as “primary” screens in that they can be generated to permit rapid development and relatively easy detection of an alteration in a molecular target which is mediated by a test compound. Moreover, the effects of cellular toxicity and/or bioavailability of the test compound can be generally ignored in the in vitro system, the assay instead being focused primarily on the effect of the drug on the molecular target as may be manifest in an alteration of binding affinity with upstream or downstream elements. Accordingly, in an exemplary screening assay of the present invention, the compound of interest is contacted with the TPL-2 polypeptide with or without a TPL-2 target polypeptide, e.g., p105 (Kieran et al, 1990, Cell 62:1007-1018, see also Ace. No. M37492) or IκB-α (Zabel et al., 1990, Cell 61:255-265) and detection and quantification of phosphorylation of TPL-2 and/or the target polypeptide is determined by assessing a compound's efficacy at inhibiting the formation of phosphorylated TPL-2 and/or a TPL-2 target polypeptide using, for example, a radioisotope. The efficacy of the test compound can be assessed by generating dose response curves from data obtained using various concentrations of the test compound. Moreover, a control assay can also be performed to provide a baseline for comparison. In another embodiment, various candidate compounds are tested and compared to a control compound with a known activity, e.g., an inhibitor having a known generic activity, or, alternatively, a specific activity, such that the specificity of the test compound may be determined. Accordingly, if desired, a general kinase inhibitor, e.g., staurosporin (see, e.g., Tamaoki et al., 1986, Biochem. Biophys. Res. Comm. 135:397-402; Meggio et al., 1995, Eur. J. Biochem. 234:317-322) or, e.g., specific kinase inhibitors such as, e.g., the commercially available inhibitors PD 98059 (a potent inhibitor of MEK, see, e.g., Dudley et al., 1995, P.N.A.S. 92:7686-7689) and SB 203580 (a potent inhibitor of p38 MAP kinase, see, e.g., Cuenda et al., 1995, FEBS Lett. 364:229-233) may be used.

In more than one embodiment of the above assay methods of the present invention, it may be desirable to immobilize the target polypeptide to facilitate separation of complexed from uncomplexed forms or accommodate automation of the assay (see, e.g., Example 4). Phosphorylation or binding of TPL-2 and a target polypeptide in the presence or absence of a test compound can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/target polypeptide fusion proteins can be adsorbed onto glutathione sepharose beads (Sigma Chemical, St. Louis, Mo.) or glutathione derivatized microtitre plates, which are then combined with the test compound and incubated under conditions conducive to phosphorylation or complex formation (e.g., at physiological conditions for salt and pH). Following incubation, the beads or microtitre plate wells are washed to remove any unbound components, the matrix immobilized in the case of beads, and the complex is measured either directly or indirectly, for example, as described above. Alternatively, the complexes can be dissociated from the matrix, and the level of target polypeptide binding or phosphorylation activity can be determined using standard techniques. Other techniques for immobilizing proteins on matrices can also be used in the screening assays of the invention

In yet another aspect of the invention, TPL-2 and a target polypeptide can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins or compounds, which bind to or interact with TPL-2 and/or a TPL-2 target polypeptide.

This invention further pertains to novel agents identified by the above-described screening assays and to processes for producing such agents by use of these assays. Accordingly, in one embodiment, the present invention includes a compound or agent obtainable by a method comprising the steps of any one of the aforementioned screening assays (e.g., cell-based assays or cell-free assays). For example, in one embodiment, the invention includes a compound or agent obtainable by any of the methods described herein.

Accordingly, it is within the scope of this invention to further use an agent, e.g., a TPL-2 molecule or compound identified as described herein in an appropriate animal model. For example, an agent identified as described herein can be used in an animal model to determine the efficacy, toxicity, or side effects of treatment with such an agent. Alternatively, an agent identified as described herein can be used in an animal model to determine the mechanism of action of such an agent. In addition, such an agent if deemed appropriate, may be administered to a human subject, preferably a subject at risk for a inflammatory disorder.

The present invention also pertains to uses of novel agents identified by the above-described screening assays for diagnoses, prognoses, and treatments of any of the disorders described herein. Accordingly, it is within the scope of the present invention to use such agents in the design, formulation, synthesis, manufacture, and/or production of a drug or pharmaceutical composition for use in diagnosis, prognosis, or treatment of any of the disorders described herein.

5. Pharmaceutical Compositions

In a preferred embodiment, there is provided a pharmaceutical composition comprising a compound or compounds identifiable by an assay method as defined in the previous aspect of the invention.

A pharmaceutical composition according to the invention is a composition of matter comprising a compound or compounds capable of modulating the p105-phosphorylating activity of TPL-2 as an active ingredient. Typically, the compound is in the form of any pharmaceutically acceptable salt, or e.g., where appropriate, an analog, free base form, tautomer, enantiomer racemate, or combination thereof. The active ingredients of a pharmaceutical composition comprising the active ingredient according to the invention are contemplated to exhibit excellent therapeutic activity, for example, in the treatment of tumors or other diseases associated with cell proliferation, infections and inflammatory conditions, when administered in amount which depends on the particular case. For example, the invention encompasses any compound that can alter TPL-2 signaling. In one embodiment, the compound can inhibit TPL-2 activity which results in the misregulation of genes involved in inflammation. For example, a compound which inhibits TPL-2 activity and thereby reduces TNF gene expression is a preferred compound for treating, e.g., inflammatory disease. In one preferred embodiment, the compounds identified according to the methods of the invention can be used to treat inflammatory disease such as, e.g., rheumatoid arthritis, multiple sclerosis (MS), inflammatory bowel disease (IBD), insulin-dependent diabetes mellitus (IDDM), sepsis, psoriasis, TNF-mediated disease, and graft rejection. In another embodiment, one or more compounds of the invention may be used in combination with any art recognized compound known to be suitable for treating the particular indication in treating any of the aforementioned conditions. Accordingly, one or more compounds of the invention may be combined with one or more art recognized compounds known to be suitable for treating the foregoing indications such that a convenient, single composition can be administered to the subject.

Dosage regima may be adjusted to provide the optimum therapeutic response. For example, several divided doses may be administered daily or the dose may be proportionally reduced as indicated by the exigencies of the therapeutic situation.

The active ingredient may be administered in a convenient manner such as by the oral, intravenous (where water soluble), intramuscular, subcutaneous, intranasal, intradermal or suppository routes or implanting (e.g. using slow release molecules). Depending on the route of administration, the active ingredient may be required to be coated in a material to protect said ingredients from the action of enzymes, acids and other natural conditions which may inactivate said ingredient.

In order to administer the active ingredient by other than parenteral administration, it will be coated by, or administered with, a material to prevent its inactivation. For example, the active ingredient may be administered in an adjuvant, co-administered with enzyme inhibitors or in liposomes. Adjuvant is used in its broadest sense and includes any immune stimulating compound such as interferon. Adjuvants contemplated herein include resorcinols, non-ionic surfactants such as polyoxyethylene oleyl ether and n-hexadecyl polyethylene ether. Enzyme inhibitors include pancreatic trypsin.

Liposomes include water-in-oil-in-water CGF emulsions as well as conventional liposomes.

The active ingredient may also be administered parenterally or intraperitoneally. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions (where water soluble) or dispersions and sterile powders for the extemporaneous preparation of sterile injectable solutions or dispersion. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants.

The prevention of the action of microorganisms can be brought about by various antibacterial and antifungal agents, for example, parabens, chlorobutanol, phenol, sorbic acid, thirmerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active ingredient in the required amount in the appropriate solvent with various of the other ingredients enumerated above, as required, followed by filtered sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active ingredient is suitably protected as described above, it may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the active ingredient may be incorporated with excipients and used in the form of ingestible tablets, buccal tablets, troches, capsules, elixirs, suspensions, syrups, wafers, and the like. The amount of active ingredient in such therapeutically useful compositions in such that a suitable dosage will be obtained.

The tablets, troches, pills, capsules and the like may also contain the following: a binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier.

Various other materials may be present as coatings or to otherwise modify the physical form of the dosage unit. For instance, tablets, pills, or capsules may be coated with shellac, sugar or both. A syrup or elixir may contain the active ingredient, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active ingredient may be incorporated into sustained-release preparations and formulations.

As used herein “pharmaceutically acceptable carrier and/or diluent” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the novel dosage unit forms of the invention are dictated by and directly dependent on (a) the unique characteristics of the active material and the particular therapeutic effect to be achieved, and (b) the limitations inherent in the art of compounding such as active material for the treatment of disease in living subjects having a diseased condition in which bodily health is impaired.

The principal active ingredients are compounded for convenient and effective administration in effective amounts with a suitable pharmaceutically acceptable carrier in dosage unit form. In the case of compositions containing supplementary active ingredients, the dosages are determined by reference to the usual dose and manner of administration of the said ingredients.

In a further aspect there is provided the active ingredient of the invention as hereinbefore defined for use in the treatment of disease either alone or in combination with art recognized compounds known to be suitable for treating the particular indication. Consequently there is provided the use of an active ingredient of the invention for the manufacture of a medicament for the treatment of disease associated with NFκB induction or repression.

Moreover, there is provided a method for treating a condition associated with NFκB induction or repression, comprising administering to a subject a therapeutically effective amount of a compound or compounds identifiable using an assay method as described above.

The invention is further described, for the purpose of illustration only, in the following examples.

### EXAMPLE 1

Identification of an Interaction Between TPL-2 and p105

In order to identify possible targets for TPL-2, a yeast two-hybrid screen is performed using an improved mating strategy (Fromont-Racine, et al., (1997) Nature Gene. 16:277-282). TPL-2 cDNA, subcloned in to the pAS2ΔΔ vector, is used as a bait to screen a human liver cDNA library (provided by Dr. Legrain, Pasteur Institute, Paris).

68 clones are obtained, positive for HIS3 selection and LacZ expression, from 22×10⁶ diploid yeast colonies plated. Interacting proteins are identified by DNA sequencing and confirmed by re-transformation into yeast.

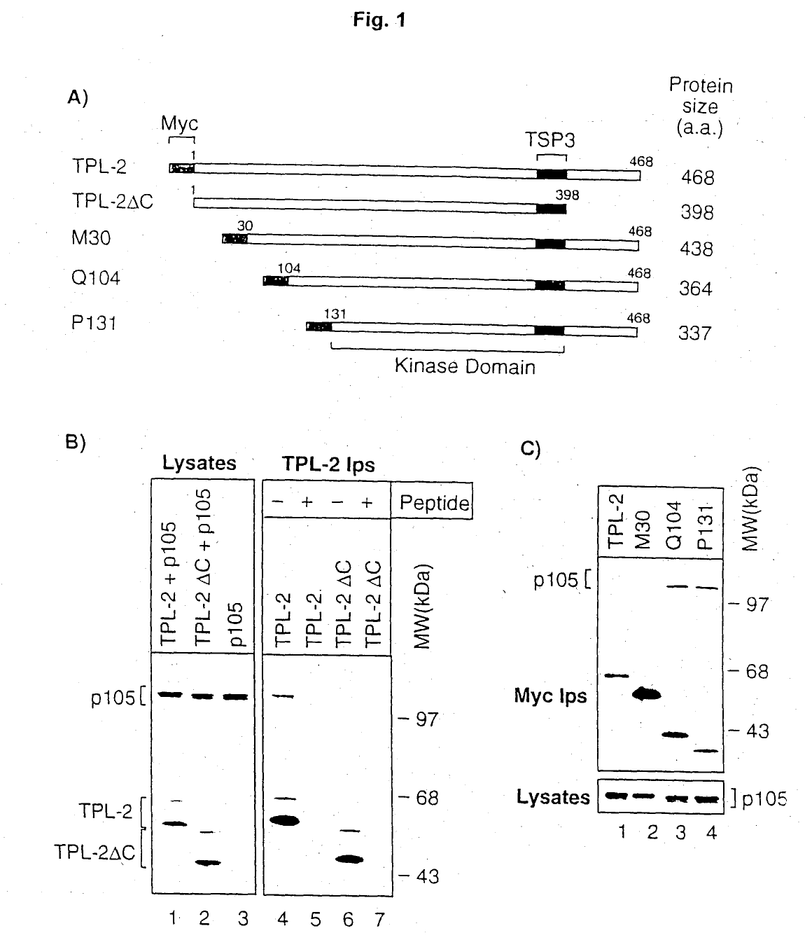

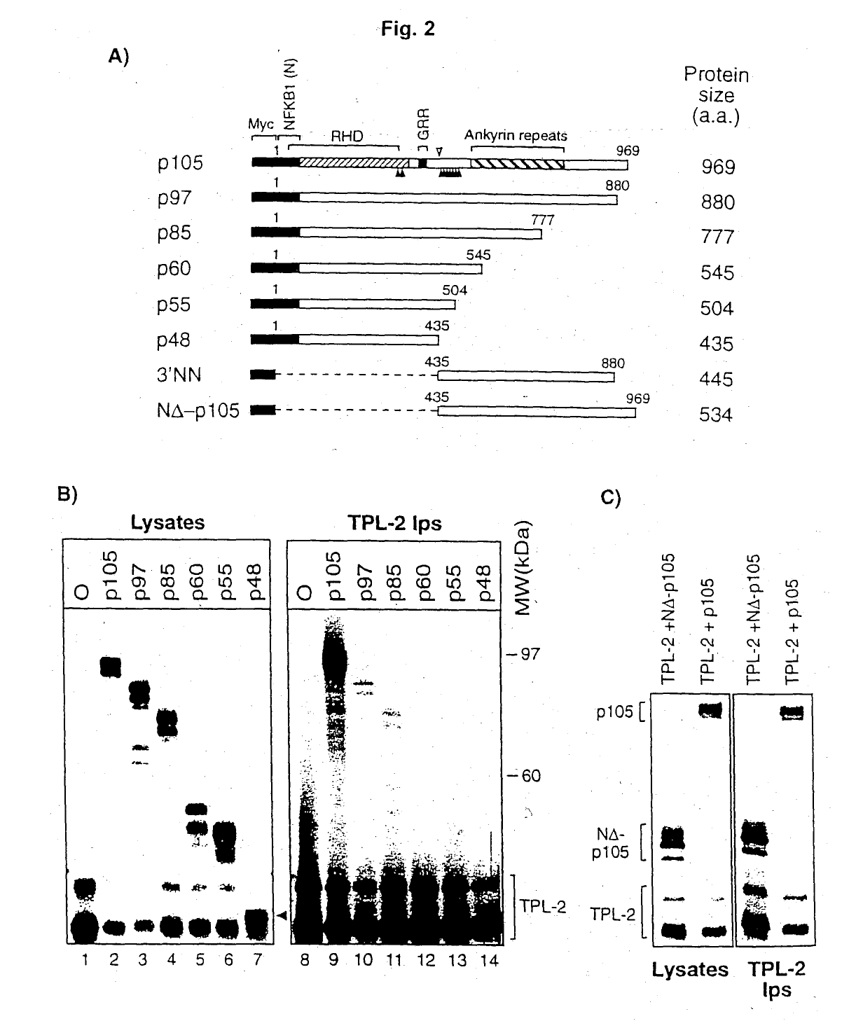

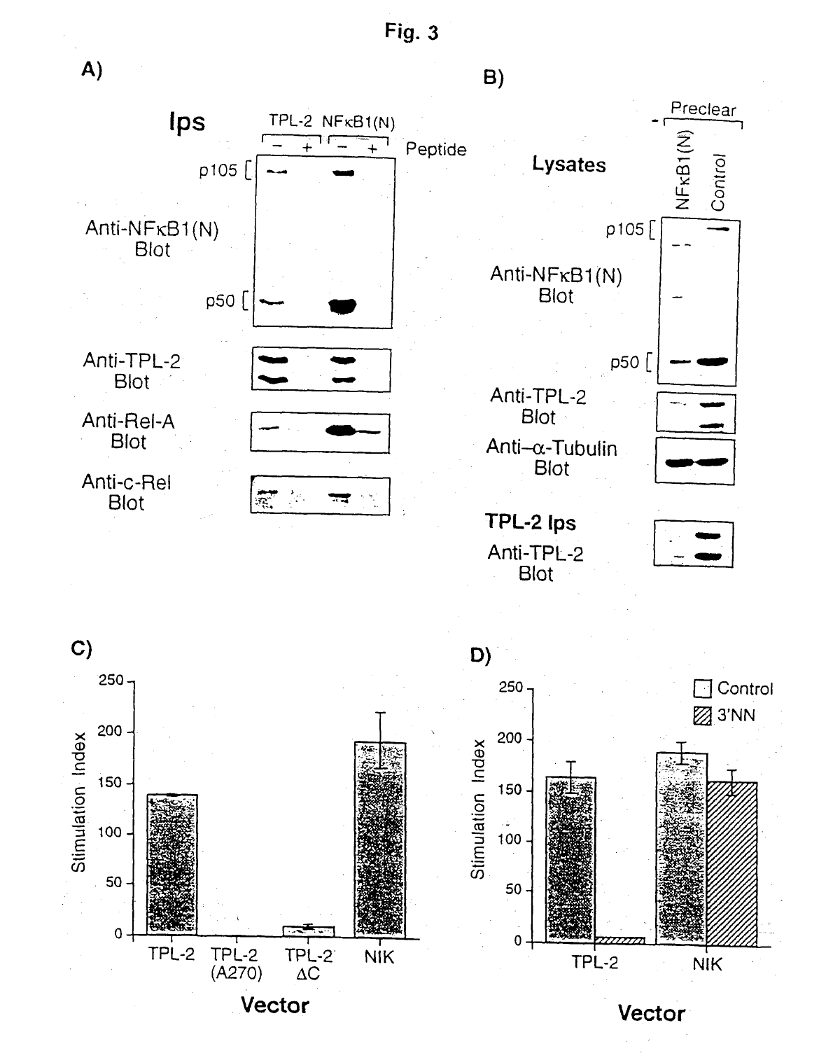

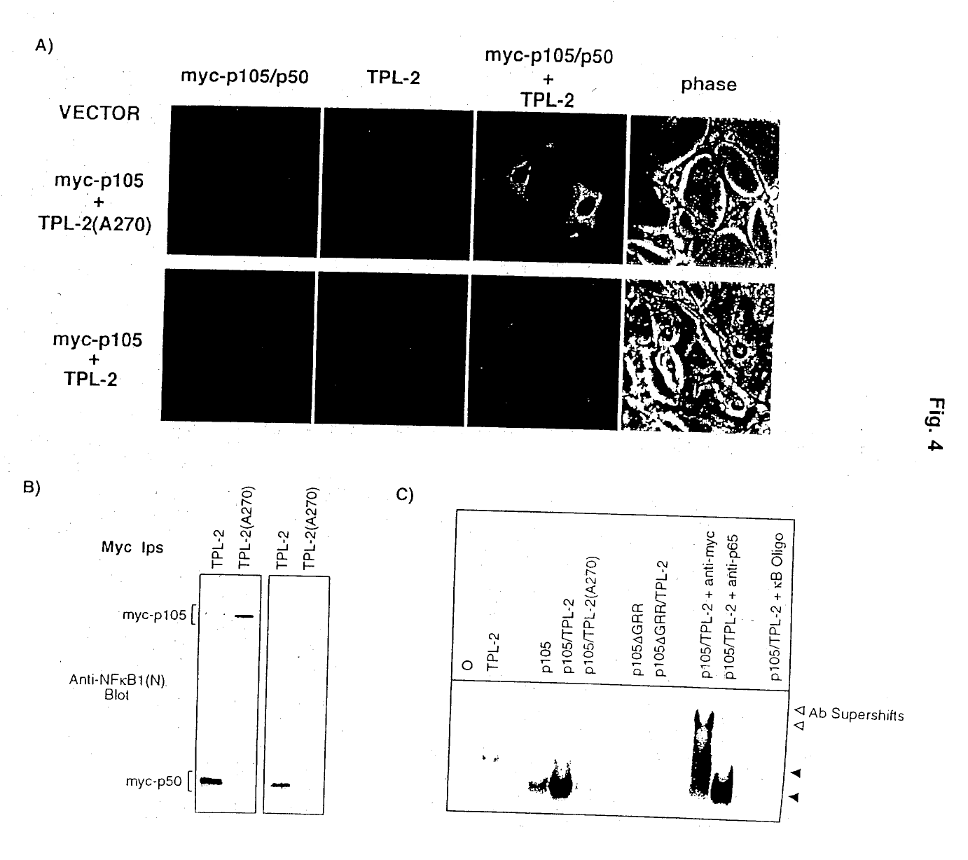

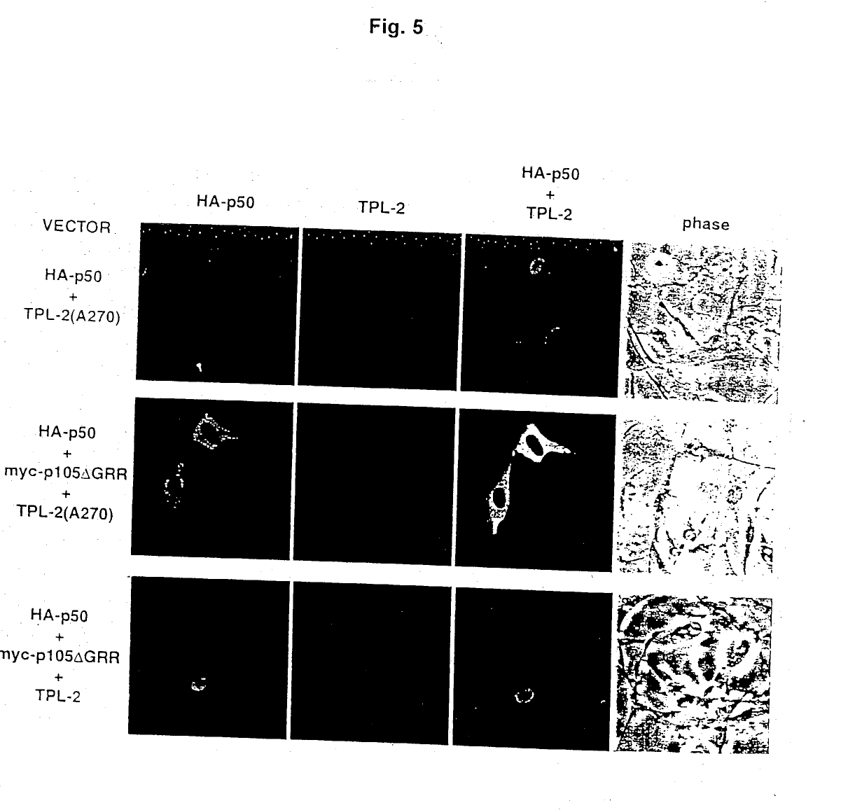

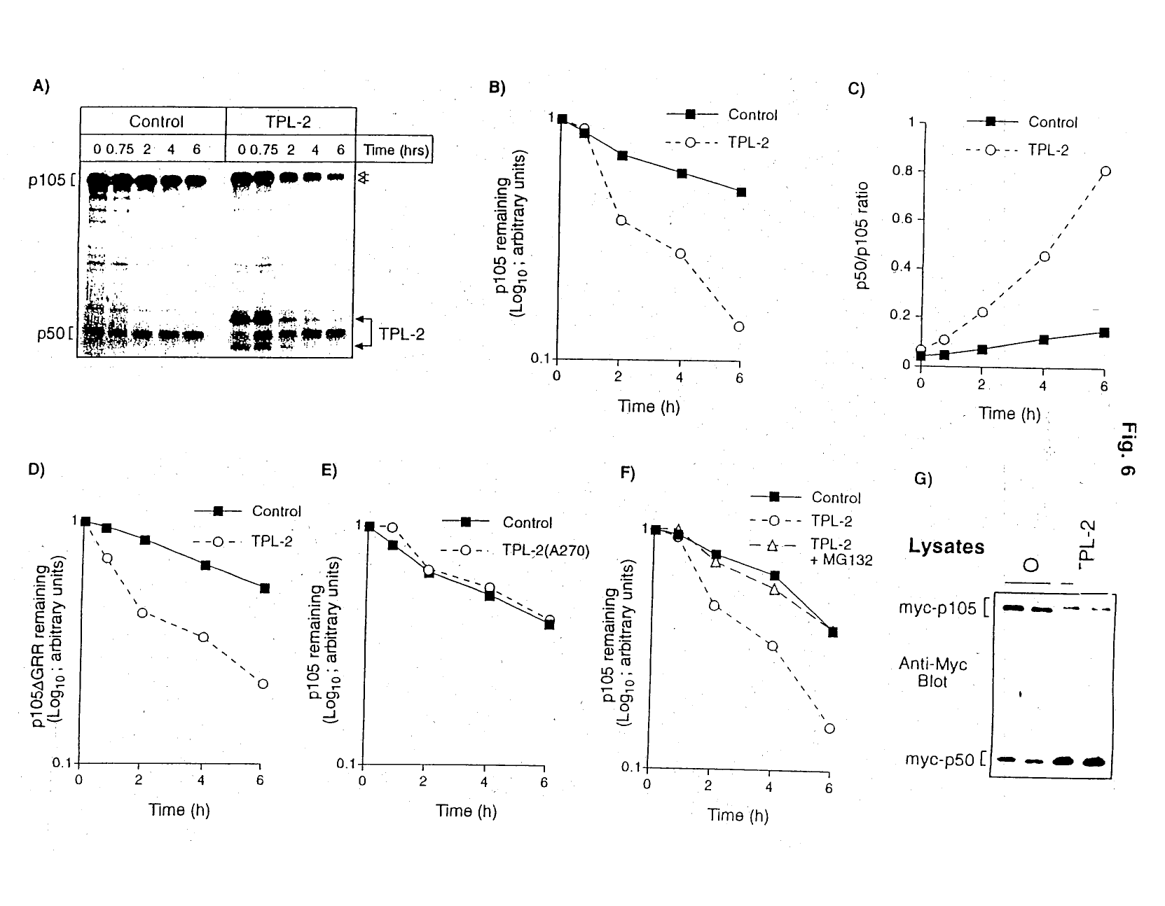

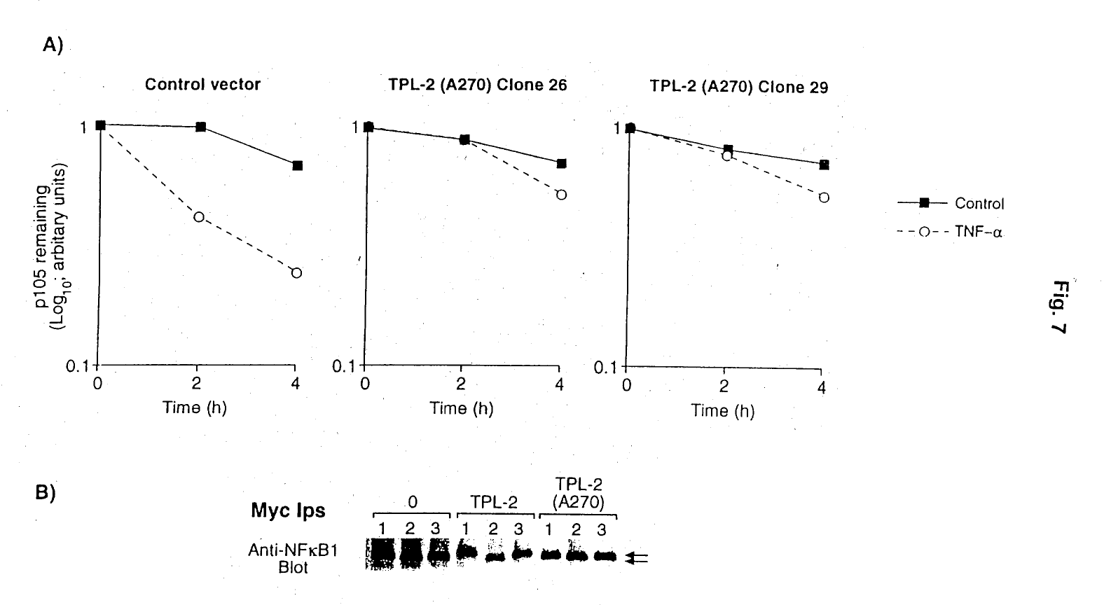

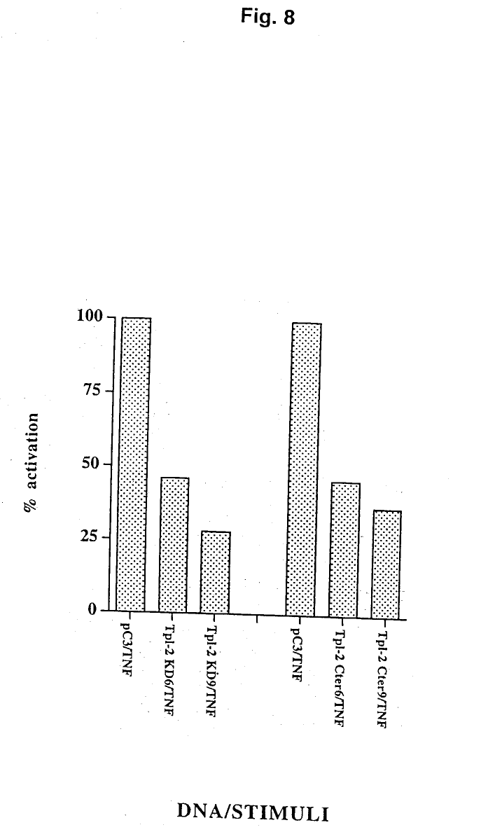

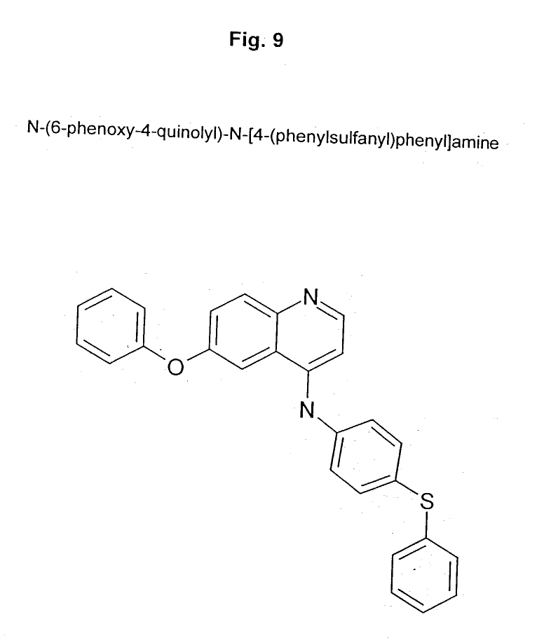

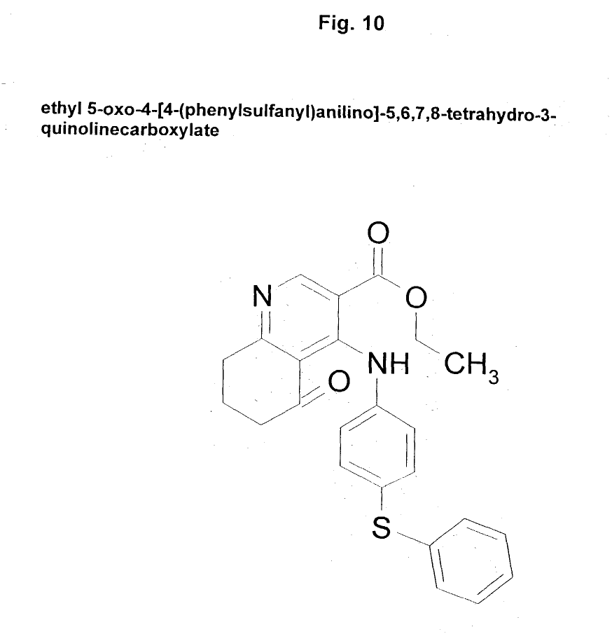

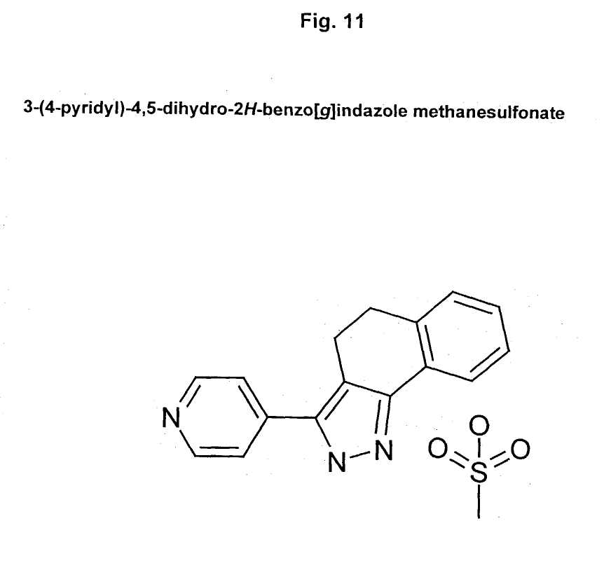

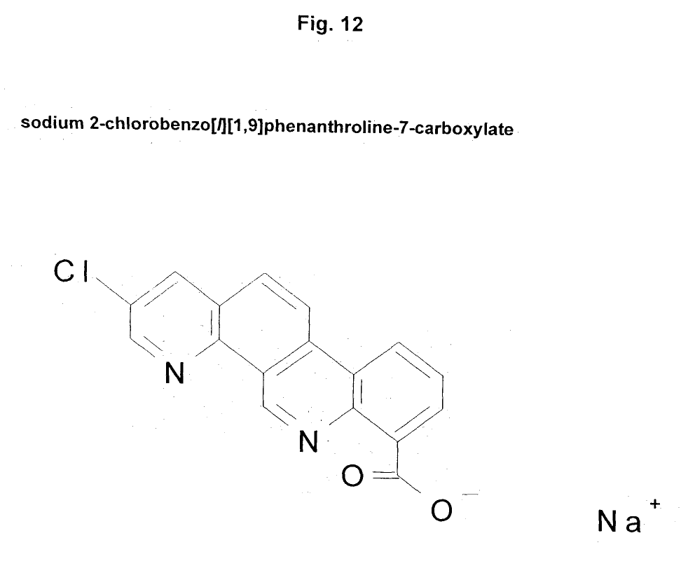

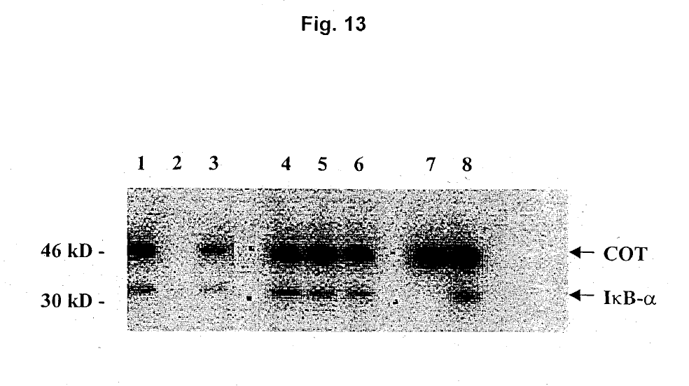

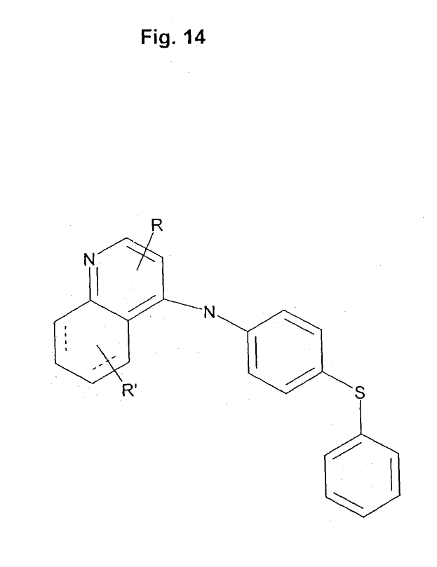

32 out of 68 positive clones obtained encode the IκB-like C-terminus of NF-κB1 p105 (FIG. 2a). Co-immunoprecipitation of p105 with TPL-2, synthesized together by cellfree translation, confirms that the two proteins interact at high stoichiometry (FIG. 1b). TPL-2 and p105 are synthesized together, and labeled with [³⁵S]-Met (Amersham-Pharmacia Biotech), by cell-free translation using the Promega TNT coupled rabbit reticulocyte system. Translated proteins are diluted in lysis buffer A (Salmeron, A., et al. (1996) EMBO J 15: 817-826) plus 0.1 mg/ml BSA and immunoprecipitated as described in the above-cited reference. Isolated proteins are resolved by SDS-PAGE and revealed by fluorography.

In experiments in which p105 is translated in excess of TPL-2, the stoichiometry of the TPL-2/p105 complex, isolated with anti-TPL-2 antibody, is estimated to be approximately 1:1. A kinase inactive mutant of TPL-2 associates with p105 at a similar stoichiometry.

To confirm the TPL-2/p105 interaction in vivo, the endogenous proteins are immunoprecipitated from HeLa cells. hnmunoprecipitation and western blotting of endogenous proteins from cell Lysates of confluent HeLa cells (90 mm dishes; Gibco-BRL), are carried out as described (Kabouridis, et al., (1997) EMBO J. 16:4983-4998), following extraction in buffer A and centrifugation at 100,000 g for 15 min. The anti-TPL-2 antibody, TSP3, has already been described (Salmeron, A., et al. (1996) EMBO J. 15: 817-826). Antibodies to NF-κB1(N) (Biomol Research labs), Rel-A (Santa Cruz) and c-Rel (Santa Cruz) are obtained from the indicated commercial suppliers. The anti-myc MAb, 9E10 (Dr. G. Evan, ICRF, London), is used for immunoprecipitation and immunofluorescence of myc-p105/myc-p50, whereas anti-myc antiserum (Santa Cruz) is used for immunoblotting. The anti-HA MAb, 12CA5, is used for immunofluorescent staining of HA-p50.

Western blotting clearly demonstrates specific co-immunoprecipitation of p105 with TPL-2 (FIG. 3a). p50, Rel-A and c-Rel also specifically co-immunoprecipitated with TPL-2. However, in vitro experiments failed to detect any direct association between TPL-2 and p50 (generated from the p48 mutant; FIG. 2b, lane 14), Rel-A or c-Rel. Thus, the p105 associated with TPL-2 in vivo is probably complexed with Rel subunits via the N-terminal Rel Homology Domain (RHD) of p105 (Ghosh, et al., (1998) Annu. Rev. Immunol. 16:225-260).

The stoichiometry of interaction of TPL-2 with p105 in vivo is investigated by immunodepletion of HeLa cell Lysates with anti-NFκB1(N) antiserum. Western blotting of anti-TPL-2 immunoprecipitates demonstrates that virtually all detectable TPL-2 is removed in NF-κB1-depleted cell Lysates (FIG. 3b, bottom panel). Immunodepletion of TPL-2 removes approximately 50% of total cellular p105. Thus, in HeLa cells, essentially all TPL-2 is complexed with a large fraction of total p105, consistent with in vitro data indicating a high stoichiometry interaction (FIG. 1, b and c).

### EXAMPLE

Analysis of TPL-2 and p105 Mutants

A. Deletion Mutants

TPL-2 deletion constructs are subcloned into the pcDNA3 expression vector (ivitrogen). Addition of an N-terminal myc epitope-tag to TPL-2 cDNA, generation of TPL-2 deletion mutants (FIG. 1a) and the TPL-2(A270) kinase-inactive mutant (untagged) are performed using PCR with the appropriate oligonucleotides and verified by DNA sequencing. Full length TPL-2 is used without a myc-epitope tag unless otherwise indicated in the figure legend. Myc-p105 deletion mutants and HA-p50, subcloned into either the pcDNA1 (Invitrogen) or pEF-BOS expression vectors, have been described previously (Watanabe, et al., (1997) EMBO J. 16:3609-3620; Fan, et al., (1991) Nature 354:395-398) with the exception of myc-NΔ-p105 which is generated by PCR and subcloned into pcDNA3. In the experiments shown in FIG. 1, untagged p105 cDNA, subcloned in the pRc-CMV expression vector (Invitrogen), is used for translation of p105 (Blank, et al., (1991) EMBO J. 10:4159-4167).

Immunoprecipitation experiments performed as described in Example 1 with deletion mutants of TPL-2 and p105 reveal that the two proteins interact through their C-ternuni (FIGS. 1 and 2). Of particular interest, an oncogenic mutant of TPL-2, TPL-2AC (Salmeron, A., et al. (1996) EMBO J. 15: 817-826), which lacks the C-terminus, does not efficiently co-immunoprecipitate with p105 (FIG. 1b, lanes 5 and 6). In addition, a GAL4 fusion of just the C-terminal 92 amino acids of TPL-2 interacts with the p105 C-terminus (residues 459 to 969) in a yeast two-hybrid assay. In vitro, TPL-2 appears to interact with two regions in the C-terminus of p105 (FIG. 2b, right panel), one in the last 89 amino acids and the other between residues 545 and 777. The isolated p105 C-terminus is sufficient to form a stable complex with TPL-2 (FIG. 2c).

B. Dominant Negative TPL-2

It is important to establish that the effects of TPL-2 expression on p105 proteolysis (see below) reflect its normal physiological function. To this end, kinase-inactive TPL-2(A270) is tested for its ability to block agonist-induced p105 degradation. 1×10⁷ Jurkat T cells are co-transfected, by electroporation, with TPL-2(A270) cDNA subcloned in the PMT2 vector (5 pg), together with the selection vector, J6-Hygro (0.5 pg). Control cells are co-transfected with PMT2 control vector and J6Hygro. Transfected cells are cloned by limiting dilution and selected for hygromycin resistance (0.5 mg/ml). Expression of TPL-2(A270) in clones generated is determined by western blotting. Pulse-chase metabolic labeling of Jurkat clones is carried out as for 3T3 cells, using 8×10⁶ cells per point.

In Jurkat T cells stably expressing control empty vector or in untransfected parental cells, TNF-a stimulates p105 degradation (FIG. 7a), consistent with an earlier study (Mellits, et al., (1993) Nuc. Acid. Res. 21, 5059-5066). However, TNF-a stimulation of Jurkat T cells which are transfected to express TPL-2(A270) has little effect on p105 turnover (FIG. 7a). Thus TPL-2 activity is required for TNF-a to induce p105 degradation, and the activity of TPL-2 may be blocked by expression of a dominant negative mutant thereof.

This result is confirmed in a further experiment, demonstrating inhibition of the transcription-activation potential of p105/TNF by dominant negative TPL-2. Jurkat T cells are transfected as above, using a TNF-induced reporter construct driving a luciferase gene. Co-expression of kinase-dead TPL-2, or the truncated C-terminus of TPL-2, which has no kinase domain, decreases luciferase gene expression markedly (see FIG. 8).

### EXAMPLE 3

Functional Interaction of p105 and TPL-2

(A) NκB Activation

To investigate whether TPL-2 activates NF-κB via p105, transiently transfected TPL-2 is initially tested for its ability to activate an NF-κB reporter gene. For NF-κB reporter gene assays, Jurkat T cells are co-transfected (Kabouridis, et al., (1997) EMBO J. 16:4983-4998) with 2,ug of a plasmid containing five tandem repeats of a consensus NF-κB enhancer element upstream of a luciferase gene (Invitrogen) together with the indicated amounts of the appropriate expression vectors. TPL-2 and NIK cDNAs are all subcloned in the pcDNA3 vector (Salmeron et al., (1996); Malintn, et al., (1997) Nature 385:540-544). The amount of transfected DNA is kept constant by supplementation with empty pcDNA3 vector. Luciferase experiments (Kabouridis, et al., (1997) are performed at least three times yielding similar results.

Expression of TPL-2 activates the reporter gene over 140-fold (FIG; 3c), a similar level to that induced by NIK, a related MAP 3K enzyme which activates NF-κB by stimulating the degradation of IκB-a (Malinin, et al., (1997); May, M. J. & Ghosh, S. (1998) Immunol. Today 19, 80-88. A kinase inactive point mutant, TPL-2(A270), has no effect on NF-κB induction. Expression of TPL-2AC, which does not form a stable complex with p105 either in vitro (FIG. 1b) or in vivo, results in only very modest activation (12-fold) of the NF-κB reporter (FIG. 3c). To confirm that TPL-2 must be complexed with p105 to efficiently activate NF-κB, a C-terminal fragment of p105, 3'NN (FIG. 2a), is co-expressed with TPL-2. This C-terminal fragment interacts with co-transfected TPL-2 in vivo, competing for binding to endogenous p105. Co-expression of 3'NN dramatically inhibits activation of the NF-κB reporter by TPL-2 but not by NIK (FIG. 3d). Together, these data indicate that transfected TPL-2 potently activates NF-κB and this appears to require direct interaction with endogenous p105. This implies that TPL-2 might directly activate p105.

(B) Nuclear Translocation of NFκB

If TPL-2 expression does indeed activate p105, nuclear translocation of NF-κB1 should result. To investigate this, an immunofluorescence assay is used in 3T3 fibroblasts, in which distinction between cytoplasm and nucleus is facile. Briefly, NIH-3T3 cells are transiently transfected with the indicated vectors and cultured on cover-slips for 24 h. Cells are then fixed, permeabilised and stained with the indicated antibodies and appropriate fluorescently-labelled second stage antibodies, as described previously (Huby, et al., (1997) J. Cell. Biol. 137, 1639-1649). A Leica TCS NT confocal microscope is used to visualize single optical sections of stained transfected cells.

In cells transfected with myc-p105 on its own or together with kinase-inactive TPL-2(A270), anti-myc staining is restricted to the cytoplasm (FIG. 4a, upper panels), consistent with the function of p105 as an IκB. Co-expression with TPL-2, however, induces an essentially quantitative shift of anti-myc staining to the nucleus (FIG. 4a, lower panels). Cell fractionation and western blotting confirm that the nuclear NF-κB signal in cells transfected with TPL-2 is myc-p50 rather than myc-p105, which is restricted to the cytoplasm (FIG. 4b). These data suggest that TPL-2 expression induces nuclear translocation of myc-p50 as a consequence either of increased processing of co-transfected myc-p105 to myc-p50, or of its degradation to release associated myc-p50.

To determine whether TPL-2 must induce p105 proteolytic processing to promote p50 nuclear translocation, 3T3 cells are transfected with a vector encoding myc-p105AGRR, which cannot be processed to myc-p50, together with HA-p50 on a separate plasmid. HA-p50 localizes in the nucleus when co-expressed with TPL-2(A270) (FIG. 5, top panels) or empty vector. Myc-p105ΔGRR retains HA-p50 in the cytoplasm of cells co-transfected with TPL-2(A270) (FIG. 5, middle panels). However, co-expression of TPL-2 with myc-p105ΔGRR induces an essentially quantitative shift of HA-p50 staining to the nucleus (FIG. 5, lower panels). Thus, TPL-2 activation of p50 nuclear translocation does not require stimulation of p105 processing to p50. These data support the position that TPL-2 induces degradation of p105 to release associated p50, or other associated Rel subunits, to translocate into the nucleus and thereby generate active NF-κB.

(C) Biological Activity of NFκB

An electrophoretic mobility shift assay (EMSA) is carried out as described (Alkalay, L, et al., (1995) Mol. Cell. Biol. 15, 1294-1301), using a radiolabelled double-stranded oligonucleotide (Promega), corresponding to the NF-κB binding site in the mouse Igic enhancer (Lenardo, M. J. & Baltimore, D., (1989) Cell 58, 227-229), to confirm that nuclear myc-p50 produced from myc-p105 in cells co-expressing TPL-2 is biologically active.

Expression of TPL-2 results in a clear increase in two kb-binding complexes (FIG. 4c, lane 2), consistent with TPL-2 activation of an NF-κB reporter gene in Jurkat T cells (FIG. 3c). Myc-p105 expression alone modestly increases binding activity of the lower κB complex (FIG. 4c, lane 3). However, co-expression of myc-p105 with TPL-2 results in a synergistic increase in binding activity of the lower κB complex (FIG. 4c, lane 4). Kinase-inactive TPL-2(A270) has no effect on κB binding activity (FIG. 4c, lane 5). A processing deficient mutant of p105, myc-p105AGRR (Watanabe, et al., (1997) EMBO J. 16:3609-3620), also fails to generate κB binding activity in the presence or absence of co-expressed TPL-2 (FIG. 4c, lane 6 and 7).

Anti-myc MAb strongly reacts with the induced lower κB complex in TPL-2 plus myc-p105 co-transfected cells, causing a supershift (FIG. 4c, lane 8). This confirms the presence of processed myc-p50 in this complex. The induced lower complex does not react with antibodies to Rel-A (FIG. 4c, lane 9) or c-Rel. Thus, co-expression of TPL-2 with myc-p105 stimulates production of active NF-κB complexes, primarily comprising dimers of myc-p50, which is overproduced in myc-p105 transfected cells. Supershift analyses of nuclear extracts from cells transfected with TPL-2 alone (FIG. 4c, lane 2) reveals that the major induced endogenous NF-κB complex is composed of p50/Rel-A dimers (FIG. 4c, lane 9).

(D) Biological Effect of TPL-2 on p105

Pulse-chase metabolic labeling is performed to determine whether TPL-2 regulates the proteolysis of myc-p105 in 3T3 fibroblasts. For pulse-chase metabolic labeling, NIH-3T3 fibroblasts are transiently transfected using LipofectAMINE (Gibco-BRL) (Huby, et al., (1997) J. Cell. Biol. 137, 1639-1649). Preparation of cytoplasmic and nuclear fractions is performed as described (Watanabe, et al., (1997)

EMBO J. 16, 3609-3620). For pulse-chase metabolic labeling, 2.7×105 3T3 cells per 60 mm dish (Nunc) are transfected with the indicated expression vectors. After 24 h, cells are washed and cultured in Met/Cys-free medium for 1 h. Cells are then labeled with 145 MBq of [³⁵S]-Met/[³⁵S]-Cys (Pro-Mix, Amersham-Pharmacia Biotech) per dish for 30 min and after washing, chased in complete medium for the indicated times. Cells are lysed in Buffer A (Salmeron et al.) supplemented with 0.1% SDS and 0.5% deoxycholate (RIPA buffer) and immunoprecipitated proteins are revealed by fluorography. MG132 proteasome inhibitor (Biomol Research labs) is added at 2O′1M during the last 15 min of the Met/Cys starvation period and is maintain throughout the chase. Labeled bands are quantified by laser densitometry using a Molecular Dynamics Personal Densitometer. All pulse-chase experiments are performed on at least two occasions with similar results.

Co-expression with TPL-2 decreases the half-life of myc-p105 from approximately 5.51 to 1.8 h (FIG. 5 a and b). Comparison of the rate of decrease of myc-p105 with that of myc-p50 production suggests that the majority of myc-p105 is simply degraded, rather than being converted to myc-p50 (FIG. 6a), as previously suggested (Lin, et al., (1998) Cell 92, 819-828). However, TPL-2 co-expression does not alter the overall rate of production of myc-p50, which is predominantly generated post-translationally from myc-p105 in these cells (FIG. 6a), rather than by the recently described co-translational mechanism (Lin et al., 1998). Since myc-p50 is generated at a similar rate in TPL-2 co-transfected cells as in control cells, but from progressively decreasing amounts of myc-p105 (FIG. 6c), this suggests that TPL-2 dramatically increases the efficiency of myc-p105 processing. TPL-2 co-expression promotes the degradation of myc-p105AGRR (FIG. 6d), similarly to wild type p105 (FIG. 6b). Kinase-inactive TPL-2(A270), however, have no detectable effect on either degradation (FIG. 6e) or processing of co-expressed myc-p105.

The effect of the peptide aldehyde MG132, a potent inhibitor of the proteasome, is determined to investigate whether myc-p105 proteolysis induced by TPL-2 is mediated by the proteasome. MG132 treatment blocks increased turnover of myc-p105 (FIG. 6f) and completely prevents production of myc-p50 in TPL-2 co-expressing cells. In conclusion, the pulse-chase metabolic labeling experiments indicate that the predominant effect of TPL-2 expression is to increase the rate of myc-p105 degradation by the proteasome. However, at the same time, the overall rate of production of myc-p50 from myc-p105 by the proteasome is not altered.

To determine the effect of TPL-2 expression on steady state levels of myc-p105/myc-p50, 3T3 cells are transiently co-transfected with the indicated vectors and lysed in RIPA buffer after 24 h. Western blots of cell Lysates are then probed with anti-myc antiserum. Bands are quantified by laser densitometry. Western blotting of lysates from the transiently-transfected 3T3 cells demonstrates that the steady-state ratio of myc-p50/myc-p105 is increased significantly by TPL-2 co-expression compared to control (FIG. 6g).

Thus, in TPL-2 transfected cells, myc-p50 is expressed in large molar excess over myc-p105 (myc-p50/myc-p105 mean=10.3+/−SE1.3; n=2), whereas in control cells myc-p105 and myc-p50 are almost equimolar (myc-p50/myc-p105 mean=0.93+/SEO.07; n=2). Myc-p50 translocates into the nucleus of TPL-2 co-transfected cells, therefore, as there is insufficient myc-p105 to retain it in the cytoplasm.

NIK phosphorylates and activates two related kinases, termed IKK-a (IKK-1) and IKK-α (IKK-2) which, in turn, phosphorylate regulatory serines in the N-terminus of IκB-α. This triggers IκB-α ubiquitination and degradation by the proteasome. To investigate whether phosphorylation causes the mobility shift in myc-p105 co-expressed with TPL-2, washed anti-myc immunoprecipitates are resuspended in buffer containing 50 mM Tris-pH7.5, 0.03% Brij-96, 0.1 mM EGTA, 1 mM DTT, 0.1 mg/ml BSA. Calf intestinal phosphatase (CIP; Boehringer-Mannheim) is added to the appropriate samples at 400 U/ml with and without the phosphatase inhibitors sodium orthovanadate (1 mM), sodium fluoride (5 mM) and okadaic acid (0.1 μm). After incubation at 37° C. for 1 h, immunoprecipitated protein is western blotted and probed with anti-NF-κB1(N) antiserum.

TPL-2 stimulation of myc-p105 degradation requires its kinase activity (FIG. 6 b and e) indicating that phosphorylation is similarly necessary for this effect. Myc-p105 co-expressed with TPL-2 is consistently found to migrate more slowly in SDS-PAGE (FIG. 6a). This TPL-2-induced mobility shift is due to myc-p105 phosphorylation, as revealed by sensitivity to in vitro treatment with phosphatases (FIG. 7b). In contrast, kinase-inactive TPL-2(A270) does not induce a mobility shift in co-expressed myc-p105 (FIG. 7b). Thus, TPL-2 stimulation of myc-p105 proteolysis correlates with its induced phosphorylation. By analogy with IκB-α, it is likely that TPL-2-induced p105 phosphorylation promotes its ubiquitination and thereby stimulates p105 proteolysis by the proteasome.

TPL-2, therefore, is a component of a novel signaling pathway which activates NF-κB by stimulating proteasome-mediated proteolysis of the NF-κB inhibitory protein, p105. TPL-2 increases the degradation of p105 whilst maintaining the overall rate of p50 production (FIG. 6a). Thus, associated Rel subunits either move into the nucleus on their own (probably as dimers) or complexed with p50 product. Since TPL-2 specifically co-immunoprecipitates with p50, Rel-A and c-Rel (FIG. 3a), it may regulate proteolysis of all the major p105 complexes present in cells (Rice, et al., (1992) Cell 71, 243-253; Mercurio, et al., (1993) Genes. Devel. 7, 705-718) Interestingly, TPL-2 is the most closely homologous kinase to NIK (Malinin, 1997), which regulates the inducible degradation of IκB-α. Therefore, two signaling pathways leading to NF-κB activation are regulated by related MAP 3K-family enzymes.

Finally, these data suggest a potential mechanism for the oncogenic activation of TPL-2, which requires deletion of its C-terminus (Ceci, et al., (1997) Gene. Devel. 11, 688700). Thus, C-terminal deletion both increases the expression of TPL-2 and releases it from stoichiometric interaction with p105 (FIG. 1b), which together may promote phosphorylation of inappropriate target proteins. These may include MEK, which is oncogenic when activated by mutation (Cowley, et al., (1994) Cell 77, 841-852), and is strongly activated by TPL-2 (Salmeron, A., et al., (1996) EMBO J. 15, 817-826).

### EXAMPLE 4

Screening Assays for Identifying Modulators of TPL-2/COT

(A) TPL-2/COT Kinase Assay Using COT Protein Immunoprecipitated From Transfected Mammalian Cells

Throughout the example, the following materials and methods are used unless otherwise stated.

Materials and Methods

Expression of COT Polypeptide in Mammalian Cells

FLAG-tagged COT protein was expressed in 293A cells by transfection. Typically, 24 h before transfection, human 293A cells (Quantum) were plated at 2×10⁶ cells per 10 cm plate. A transfection mixture was prepared comprising 60 μl Lipofectamine (Gibco) and 800 μl Optimem (Gibco) in 15 ml tube. In. a separate tube, 8 μg of DNA encoding a FLAG-tagged COT(30-397) gene in a pcDNA vector was added to 800 μl Optimem. The contents of each tube were then mixed gently with a pipette, and allowed to incubate at room temperature for 25 min. Cells were washed once with Optimem and incubated with the transfection mixture and 6.4 ml of Optimem and allowed to incubate 5 h at 37° C. and 5% CO₂. Cells were then incubated with 8 ml DMEM+10% FBS+L-glutamine on day 1, DMEM+5% FBS+L-glutamine on day 2 and harvested 48 h post-transfection.

Immunoprecipitation of FLAG-tagged COT Protein

Transfected 293A cells expressing FLAG-COT (30-397) were lysed on ice for 15 min in lysis buffer (1% Triton X-100, 50 mM Tris-HCl pH 7.5, 150 mM NaCl, 1 mM EDTA, 1 mM EGTA, 20 mM NaF, 10 mM Na₄P₂O₇, 50 mM Na₃VO₄ plus Complete protease inhibitors (Boehringer)) and lysates were centrifuged (14,000 rpm for 10 min at 4° C.) and supernatants were collected. Immunoprecipitations were performed using FLAG Ab gel (Sigma) at 50 μg Ab per ml of lysate for 3 h at 4° C. with mixing. Gel beads were washed at 4° C. twice with lysis buffer and then twice with wash buffer (50 mM Tris-HCl pH 7.5, 100 mM NaCl, 0.1 mM EGTA and 1 mM DTT). Gel beads were then resuspended in wash buffer and aliquoted into tubes for various kinase reactions.

TPL-2/COT Kinase Assay and Inhibitor Screening

A TPL-2/COT kinase assay was used to screen various candidate TPL-2/COT kinase inhibitors. The kinase assay was performed a follows. A TPL-2 kinase (i.e., FLAG-COT (30-397)) bound on gel beads was incubated with 2 μg of a target polypeptide substrate (i.e., GST IκB-α (1-50) (Boston Biologicals) in kinase buffer (50 mM Tris-HCl pH 7.5, 10 mM MgCl₂, 1 mM EGTA, 2 mM DTT and 0.01% Brij 35) in the presence of an appropriate radiolabel (30 μM ATP and 5 μCi γ-³²P-ATP (Amersham)) for 10 min at 25° C. Reactions were performed in the presence or absence of candidate compounds for inhibitor activity that were prepared as 10 mM stock solutions in 100% DMSO. The test compounds were added to the kinase reaction mixture immediately before addition of γ-⁼P-ATP. Reactions were stopped by the addition of 5×SDS sample buffer, heating at 100° C. for 3 min and supernatants were collected using centrifugation. The autophosphorylation of COT and phosphorylation of the target polypeptide, i.e., GST-IκB-α were analyzed by gel electrophoresis (10% SDS-PAGE) followed by transfer to nitrocellulose membranes and autoradiography. As a control to confirm equivalent levels of FLAG-COT(30-397) and GST-IκB-α proteins were used in the different kinase reactions and also equivalent gel loading, immunoblots were performed with anti-FLAG and anti-GST antibodies, respectively, on the same membranes used for autoradiography. Inhibition of COT kinase activity, either autophosphorylation activity or phosphorylation of the target polypeptide GST-IκB-α was quantitated by scanning of autoradiographs (FIG. 13). Compounds that altered the level of these activities were further analyzed as described below.

TPL-2/COT Kinase Assay Using Baculovirus-expressed Recombinant COT Protein

As similarly described above, a TPL-2 polypeptide expressed in insect cells was tested for kinase activity using a target polypeptide in the presence or absence of a candidate modulator compound. In this assay, the TPL-2 kinase, i.e., COT (30-397) was prepared from insect cells infected with a baculovirus expressing the COT kinase using standard techniques. The TPL-2 kinase (100 ng at 5 μg/ml in 50 mM Tris-HCl pH 8.0) was incubated with a target polypeptide comprising a model p105 protein (i.e., 1 μg of GST-P105_{Δ1-497} at 1.4 mg/ml in PBS) in the presence or absence of a test compound and in the presence of a radiolabel ([³³P]-γ-ATP 3×stock: 60 μM cold ATP with 50 μCi/ml [³³P]-γ-ATP) in kinase assay buffer (50 mM Tris-HCl pH 7.5, 10 mM MgCl₂, 1 mM EGTA, 2 mM DTT, 0.01% Brij 35, 5 mM β-phosphoglycerol). In addition, this assay was performed in the absence of a target polypeptide (i.e., the model p105 polypeptide) to determine if any of the test compounds altered TPL-2 autophosphorylation activity.

The assay was performed in 96-well plates to allow for the efficient screening of a large number of compounds. For example, typically 10 μl of kinase and substrate were incubated per well in the 96 well plate in the presence of 10 μl of compound, 10 μl of [³³P]-γ-ATP, and incubated at 25° C. for 30 min. The reaction was stopped with 100 μl of 5 mM ATP in 75 mM H₃PO₄. A transfer of 120 μl of each reaction mixture to a 96-well phosphocellulose membrane filter plate was then conducted, incubated at 25° C. for 30 min, washed (6× with 100 μl of 75 mM H₃PO₄ per well), and assayed (using 25 μl of scintillation cocktail) for resultant kinase activity as a function of recovered labeled protein measured in scintillation counter.

Using the above assays, several compounds able to modulate TPL-2 kinase activity were identified from a chemical library selected by molecular modeling as containing potential ATP-competitive TPL-2 kinase inhibitors. The identified compounds showed an effect on COT-mediated phosphorylation of the IκB-α target polypeptide as represented by GST-IκB-α.

TPL-2/COT Kinase Modulators

Compounds showing an effect on TPL-2 were initially screened for inhibition of kinase activity at 100 μM concentration in duplicate. An example of TPL-2 kinase inhibitor screening data for selected compounds is shown in FIG. 13. To determine if the compounds being tested were specific inhibitors of TPL-2 or general kinase inhibitors, kinase inhibitors with known specificity were also tested in parallel. The general kinase inhibitor, staurosporine, the MEK inhibitor PD98059, and the p38 MAP kinase inhibitor SB 203580 showed little or no inhibitory activity on COT autophosphorylation and phosphorylation of a COT target (i.e., IκB-α). In contrast, each of the test compounds showed varying levels of specific inhibitory activity (see FIG. 13). Active compounds that inhibited TPL-2 activity >50% at 100 μM, as compared to control kinase reaction containing DMSO vehicle only (5% final concentration), were retested at three concentrations, 100 μM, 10 μM and 1 μM, to determine IC50 values for TPL-2 inhibition. TPL-2 inhibitors that were identified include N-(6-phenoxy-4-quinolyl)-N-[4-(phenylsulfanyl)phenyl]amine] with IC50=50 μM, ethyl 5-oxo-4-[4-(phenylsulfanyl)anilino]-5,6,7,8-tetrahydro-3-quinolinecarboxylate with IC50=10 μM, 3-(4-pyridyl)-4,5-dihydro-2H-benzo[g]indazole methanesulfonate with IC50=100 μM and sodium 2-chlorobenzo[l][1,9] phenanthroline-7-carboxylate with IC50=100 μM. The chemical structure for each of these compounds is shown in FIGS. 9-12.

### EXAMPLE 5

Characterization of TPL-2 Interaction with p105′

Throughout example 5, the following materials and methods are used unless otherwise stated.

(5.1) Materials and Methods

(5.1.a) Generation of p105 and TPL-2 cDNA constructs

All HA epitope-tagged p105 (HA-p105) cDNAs were cloned into the pcDNA3 vector (Invitrogen). Wild type HA-p105, HA-p105₁₋₈₀₂, HA-p105_{ΔDD}, containing an internal deletion of the death domain (residues 803-893) and HA-p105_{L842A}, containing a point mutation in the p105 DD, have been described previously (Beinke, S., et al. (2002) J. Biol. Chem. 227: 24162-24168, Salmeron et al., (2001) J. Biol. Chem. 276: 22215), as have Myc-tagged and untagged versions of TPL-2 and TPL-2ΔC(3). HA-p100, HA-p105_{Δ498-541}, HA-p105_{Δ498-541; ΔDD}, HA-p105_{Δ498-541; L842A} and the panel of N-term deletion mutants of Myc-TPL-2ΔC were all generated using PCR and verified by DNA sequencing. Myc-Raf1(CAAX) in the pEXV expression vector was kindly provided by Chris Marshall (Cancer Research UK, London) (Leevers, S. J., et al (1994) Nature 369: 411-414).

(5.1.b) Expression and Purification of Recombinant p105 Protein

To generate recombinant p105 protein, p105 cDNA sequences were cloned into pGEX-2T or pGEX-6P-1 (Amersham Pharmacia Biotech). GST fusion proteins were expressed at 30° C. in the BL21DE3 E. coli strain and purified by affinity chromatography on GSH Sepharose 4B (Amersham Pharmacia Biotech). The p105 fragments were then cleaved from GST with either thrombin (Calbiochem) or PreScission Protease (Amersham Pharmacia Biotech) and further purified by gel filtration on a Superdex column (Amersham Pharmacia Biotech). Resulting proteins, which were over 95% pure as judged by Coomassie Brilliant blue (Novex) staining of SDS-PAGE gels, were concentrated by ultrafiltration and stored at −80° C. Expected masses of all proteins were confirmed by mass spectrometry.

(5.1.c) Antibodies used for Western analysis and Immunoprecipitation of p105 and TPL2

Antibodies used for immunoprecipitation and Western blotting of HA and Myc epitope-tagged proteins have been described previously (Beinke, S, et al. (2002) J. Biol. Chem. 277: 24162-24168). Endogenous p105 was immunoprecipitated with a rabbit antibody raised to a synthetic peptide corresponding to the C-terminal 17 amino acids of murine NF-κB1 p105 and Western blotted with p105C antibody (Salmeron et al., (2001) J. Biol. Chem. 276: 22215). Endogenous TPL-2 was immunoprecipitated with 70 mer anti-TPL-2 antibody, which was raised in rabbits to a synthetic peptide corresponding to the C-terminal 70 amino acids of TPL-2, and Western blotted with TSP3 anti-TPL-2 antibody (Salmeron, A., et al (1996) EMBO J. 15: 817-826). Anti-MEK-½ and anti-phospho-MEK-½ antibodies were purchased from Cell Signaling Technology (USA). Tubulin and actin were used as loading controls for cell lysates and were detected on Western blots with TAT-1 anti-tubulin mAb (kindly provided by Keith Gull, University of Manchester, UK) and a commercial anti-actin mAb (Sigma-Aldrich), respectively.

(5.1.d) Protein Analyses

HEK 293 and 3T3 cells (3-5×10⁵ cells per 60 mm diameter Nunc dish) were transiently transfected using LipofectAMINE (Life Technologies, Inc.) and cultured for a total of 24-48 h, as described previously (Beinke, S, et al. (2002) J. Biol. Chem. 277: 24162-24168). Cell lysates were prepared using Buffer A (Beinke, S, et al. (2002) J. Biol. Chem. 277: 24162-24168). Immunoprecipitation and Western blotting of transfected proteins were carried out as described (Kabouridis, P. S., et al. 1997. EMBO J. 16:4983-4998), using the indicated antibodies.

To determine the turnover of Myc-TPL-2, 293 cells were transfected as above. After 24 h culture, cells were washed in phosphate-buffered saline and incubated for 1 h in methionine/cysteine-free minimal essential Eagle's medium (Sigma). Cells were pulse-labeled with 2.65 MBq of [³⁵S]methionine/[³⁵S]cysteine (Pro-Mix; Amersham Pharmacia Biotech) for 30 min and chased for the times indicated in DMEM+2%FCS. Cells were lysed n Buffer A supplemented with 0.5% deoxycholate and 0.1% SDS (RIPA buffer). Myc-TPL-2 was isolated by immunoprecipitation with anti-Myc mAb and labeled bands revealed by autoradiography after 10%-acrylamide SDS-PAGE.

TPL-2 was synthesised in vitro and labeled with [³⁵S]methionine/[³⁵S]cysteine (Pro-Mix; Amersham Pharmacia Biotech) by cell-free translation (Promega TNT-coupled rabbit reticulocyte system). Translated proteins were diluted by buffer (250 mM NaCl, 0.01% NP-40, 50 mM HEPES-pH7.9, 50 mM EDTA) prior to incubation with GST fusion proteins and precipitated as described (Salmeron, A., et al (1996) EMBO J. 15: 817-826).

(5.1.e) RNA Isolation and RT-PCR Analysis

Total RNA from NF-κB1-deficient or wild type 3T3 fibroblasts was isolated using Trizol reagent (Invitrogen). Semi-quantitative RT-PCR was performed utilizing the QIAGEN OneStep RT-PCR kit. Primer pairs used were as follows: (5′) primer 5′-CATTGCTGATTCATCATGC-3′ (SEQ ID) No. 6) and (3′) primer 5′-ACTGGGCTCATACACTGC-3′ (SEQ ID No. 7) for TPL-2; (5′) primer 5′-GGCGGCTTGGTGACTCTAGATA-3′ (SEQ ID No. 8) and (3′) primer 5′-GCTCGGGCCTGCTTTGAACAC-3′ (SEQ ID No. 9) for 18SrRNA. The lengths of the TPL-2 and 18SrRNA amplicons were 1169 and 560 bp, respectively. PCR products were visualized on 1.5% agarose gels and TPL-2/18SrRNA ratios determined using a densitometric imager (Kodak ID 3.5).

(5.1.J) Proteolytic Mapping

0.8 nmol of p105₄₉₈₋₉₆₉ protein was digested at RT for 60 min with trypsin (1/250 of total protein concentration; Roche Diagnostics GmbH) in the absence or presence of 1.6 nmol of a synthetic peptide corresponding to the C-terminal 70 amino acids of TPL-2 (purchased from Dr .W. Mawby, University of Bristol, UK). Samples were then resolved by 10% Bis-Tris gel electrophoresis and Western blotted onto PVDF membrane (Millipore Corp. USA). Bands were visualized by Coomassie Brilliant Blue (Novex) staining and the major differential cleavage products identified by Edman degradation (N-terminal 6 amino acids). For mass spectroscopy, p105₄₉₈₋₉₆₉ protein was digested with trypsin+/−TPL-2 C-terminal peptide for 60 min. Protein molecular weights were determined using a Platform electrospray mass spectrometer (Micromass, Manchester, UK).

(5.1. g) Surface Plasmon Resonance

Interaction between the TPL-2 C-terminus and recombinant p105 proteins was measured by surface plasmon resonance using the BIAcore system (BIAcore AB, Uppsala, Sweden; (Szabo, A., et al. (1995) Curr. Opin. Struct. biol. 5: 699-705)). The C-terminal 70 amino acids of TPL-2 (TPL-2₃₉₈₋₄₆₇) were synthesised as a biotinylated peptide (conjugated to the equivalent of Cys398 of full length TPL-2) and immobilized on a Streptavidin-coated sensor surface (Sensorchip SA, BIAcore AB). Recombinant p105 proteins were then injected at different concentrations and resonance signals monitored. Dissociation at 25° C. was measured by flushing the TPL-2/p105 complexes with HBS-P buffer (10 MM HEPES-pH7.4, 150 mM NaCl, 0.005% (v/v) polysorbate 20; BIAcore AB). Data were evaluated using BIAevaluation software, version 3.0.2.

(5.1.h) MEK Kinase Assays

Myc-TPL-2 or Myc-TPL-2ΔC were isolated by immunoprecipitation from lysates of transiently transfected 293 cells, as described above. immunoprecipitates were washed four times in Buffer A, followed by two washes in kinase buffer (50 mM Tris-pH7.5, 150 mM NaCl, 5 mM β-glycerophosphate, 2 mM dithiothreitol, 0.1 mM sodium vanadate, 10 mM MgCl₂, 1 mM EGTA, 0.01% Brij35). Beads were then resuspended in 50μl of kinase buffer plus the indicated amounts of recombinant p105 protein and incubated at 4° C. for 30 min. 1 μg kinase inactive GST-MEK1(K207A) protein (Upstate Biotechnology) was then added in 50 μl kinase buffer plus 2 mM ATP and incubated a further 30 min at RT. The supernatant was removed, mixed with an equal volume of 2×sample buffer, Western blotted after 8%-acrylamide SDS-PAGE and probed with anti-phospho-MEK-½ antibody. Immunoprecipitated Myc-TPL-2 or Myc-TPL-2ΔC was eluted with Laemlli sample buffer from the remaining anti-Myc mAb beads and quantified by Western blotting.

(5.2) TPL2 Kinase Domain Interacts with the p105 Death Domain

(5.2.a) Previous binding experiments with in vitro translated protein indicate that the C-terminus of TPL-2 is required for efficient interaction with p105 (Belich, M. P. et al (1999) Nature 397: 363-368). However, very low levels of C-terminally truncated TPL-2 (TPL-2ΔC) do co-immunoprecipitate with p105 in vitro, which are revealed only after long exposure of autoradiographs. To investigate the role of the TPL-2 C-terminus in binding to p105 in more detail, it was important to determine whether TPL-2ΔC associated with p105 in vivo. To do this, 293 cells were transiently co-transfected with expressionvectors encoding HA-p105, TPL-2 or TPL-2ΔC which lacks the C-terminal 70 amino acids (Belich, M. P. et al (1999) Nature 397: 363-368). Anti-HA immunoprecipitates and cell lysates were Western blotted for HA-p105 and TPL-2. The amounts of vector encoding TPL-2 or TPL-2ΔC were adjusted to achieve equal protein expression in lysates +/−HA-p105. TPL-2ΔC was clearly detected in anti-HA-immunoprecipitates, although at slightly lower levels than full length TPL-2 . Thus, TPL-2 can bind to p105 independently of its C-terminus in vivo.

(5.2.b) To identify which additional region(s) of TPL-2 is involved in interaction with p105, 293 cells were co-transfected with vectors encoding HA-p105 and a panel of N-terminal deletion mutants of TPL-2ΔC including M30 TPL-2ΔC, Q104 TPL-2ΔC and P131 TPL-2ΔC. M30 corresponds to the natural internal initiation site(Aoki, M., et al (1993) J. Biol. Chem. 268: 22723-22732). Immunoprecipitation and Western blotting revealed that the N-terminus of TPL-2 was not required for binding to HA-p105 in 293 cells. Taken together with previous in vitro experiments (Belich, M. P. et al (1999) Nature 397: 363-368), this suggests that both the TPL-2 kinase domain and C-terminus are involved in interaction with p105.

(5.2.c) To characterize the binding site for TPL-2ΔC on p105, a panel of C-terminal deletion mutants of HA-p105, including HA-p105 (1-892), HA-pb105 (1-802), HA-p105 (1-969, Δ803-893) and HA-p105 (1-969, L842A) were individually co-expressed with TPL-2μC in 293 cells; and association determined by immunoprecipitation and Western blotting. TPL-2ΔC interacted with HA-p105₁₋₈₉₂, which lacks the PEST region, to a similar degree to full length HA-p105. However, TPL-2AC did not interact with three mutants HA-p105₁₋₈₀₂, HA-p105_{ΔDD} or HA-p105_{L}842A, which lack a functional p105 death domain (DD). Thus, the p105 DD appears to be essential for interaction with TPL-2ΔC. TPL-2 was synthesized and labeled with [³⁵S]methionine/[³⁵S]cysteine by in vitro cell-free translation. Pull-downs were then performed with GST-p105₈₀₈₋₈₉₂ or GST control. Isolated protein was resolved by SDS-PAGE and revealed by fluorography. The GST-p150DD fusion protein also associated with in vitro translated TPL-2 suggesting a direct interaction between TPL-2 and the p150 DD. Consistent with this notion, a GAL4 binding domain-TPL-2 fusion protein associates with the isolated p105 DD (p150 residues 808-892) fused to the GAL4 activation domain in a yeast two-hybrid assay. These data suggest that the p105 death domain is the binding site for the TPL-2 kinase domain.

(5.3) Identification of the Binding Site on p105 for the C-terminus of TPL2

(5.3.a) The C-terminal 70 amino acids of TPL-2 are required for efficient interaction with the C-terminal half of p105 in vitro (Belich, M. P. et al (1999) Nature 397: 363-368). To identify the actual binding site on p105 for the TPL-2 C-terminus, recombinant protein encoding the C-terminus of p105 (residues 498-969) was digested with trypsin for 60 min, in the presence and absence of a synthetic peptide corresponding to the C-terminus of TPL-2 (residues 398-467). In the absence of TPL-2 peptide, a 16 kDa fragment accumulated in the digest, which was identified by N-terminal sequencing and mass spectrometry to correspond to amino acids 535-684 of p105. A larger fragment, with an apparent MW of 20 kDa, was generated in the presence of TPL-2 peptide, which was identified as p105 amino acids 497-683. Thus, the binding of TPL-2 peptide prevents access of the protease to target sites within the region 498-541 of p105 and suggests that this region constitutes an important component of the binding site for the C-terminus of TPL-2.

(5.3.b) In order to obtain corroborating evidence for the TPL-2 C-terminus binding site on p105, surface plasmon resonance experiments were carried out using the panel of recombinant p105 proteins including p105 1-969, p105 498-969, p105 498-806, p105 497-782 and p105 541-802. These data revealed that p105₅₄₁₋₈₀₂ did not detectably bind to the TPL-2 peptide, whereas recombinant p105 proteins that included the segment 498-541 bound to the TPL-2 peptide with nanomolar affinity constants. Taken together, the proteolytic mapping data and binding experiments strongly suggest that residues 498-541 constitute at least part of a high affinity binding site for the C-terminus of TPL-2.

(5.4) The p105 Death Domain and p105 Residues 498-541 are Both Required for Optimal TPL2 Binding

To determine the relative contribution of the two TPL-2 binding sites on p105, additional HA-p105 constructs were generated with residues 498-541 deleted alone (HA-p105_{Δ498-541}) or in combination with deletion (HA-p105_{Δ498-541; ΔDD}) or point mutation (HA-p105_{Δ498-541; L842A}) of the p105 DD. These p105 constructs and HA-p105 constructs in which the p105 DD alone was mutated (HA-p105_{ΔDD} and HA-p105_{L842A}) were individually co-expressed with wild type TPL-2 in 293 cells and immunoprecipitated from cell lysates with anti-HA antibody. The extent of co-immunoprecipitation of TPL-2 was reduced by deletion or point mutation of the DD compared with wild type HA-p105. Deletion of residues 498-541 of p105 reduced association with TPL-2 to a similar extent. However, no association of TPL-2 was detected with either of the double binding site mutants. Control experiments demonstrated that all of the HA-p105 mutants were cytoplasmic, similar to wild type, indicating that altered subcellular localization did not contribute to their differential association with TPL-2. Thus, each of the identified binding sites makes a substantial contribution to TPL-2 association with p105 in vivo and both are required for optimal complex formation. This implies that the binding affinity of TPL-2 kinase domain for the p105 DD is of a similar order to that observed between the TPL-2 C-terminus and residues 498-541 of p105 (nanomolar range). The overall affinity of TPL-2 for p105 therefore might be very high (perhaps picomolar), consistent with the finding that all of the cellular pool of TPL-2 is complexed with p105 in HeLa cells (Belich, M. P. et al (1999) Nature 397: 363-368).

(5.5) Interaction with Both Binding Sites on p105 Regulates the Metabolic Stability of TPL-2

(5.5.a) To demonstrate the role of p105 for TPL-2 stability in cells, HEK 293 cells were transiently co-transfected with a fixed quantity of vector encoding TPL-2 and increasing amounts of HA-p105 vector. Expression of HA-p105 resulted in a concentration dependent increase in the amount of TPL-2 detected in cell extracts compared with empty vector (EV) co-transfected cells. In contrast, co-expression with HA-p100, which does not associate with TPL-2, had little effect on TPL-2 protein levels. Semi-quantitative PCR demonstrated that co-expression of HA-p105 did not alter the level of Myc-TPL-2 mRNA. Thus the effect of HA-p105 on steady state levels of TPL-2 protein was not due to increased transcription from the TPL-2 expression vector, but rather suggests that HA-p105 association stabilizes TPL-2 protein. This was confirmed by pulse-chase metabolic labeling, which demonstrated that HA-p105 co-expression increased the half-life of TPL-2.

(5.5.b) The effect of each of the individual binding sites on p105 for stability of TPL-2 protein in 293 cells was also investigated. Co-expression of HA-p105 containing deletion or point mutation of the DD increased the steady-state level of TPL-2 to a lesser extent than co-expression with wild type HA-p105. Similarly, HA-p105 lacking residues 498-541 resulted in lower levels of TPL-2. Moreover, HA-p105 mutants lacking both binding sites had no effect on the levels of co-transfected TPL-2 protein. Pulse-chase metabolic labeling experiments confirmed that HA-p105_{Δ498-541; ΔDD} did not alter the half life of co-expressed TPL-2. Thus, the optimal effect of p105 on TPL-2 protein stabilization requires association via both binding sites.

(5.5.c) To determine whether p105 regulates the steady-state levels of endogenous TPL-2 protein, cell lysates were prepared from 3T3 fibroblasts derived from wild type and NF-κB1-deficient mice. TPL-2 was readily detected by Western blotting of anti-TPL-2 immunoprecipitates from wild type fibroblasts and was associated with endogenous p105. In contrast, no TPL-2 was detected in the NF-κB1 knockout fibroblasts, although semi-quantitative RT-PCR demonstrated that these cells expressed similar levels of TPL-2 mRNA to wild type cells. Thus, NF-κB1 expression appears important for the maintainance of steady-state levels of endogenous TPL-2 protein, consistent with the results of the 293 pulse-chase experiments.

(5.6) Interaction with the p105 Death Domain Inhibits TPL-2 MEK Kinase Activity

(5.6.a) To demonstrate that TPL-2 kinase activity is regulated by its association with p105, Myc-TPL-2 was purified by immunoprecipitation from transiently transfected 293 cells and its kinase activity assayed in vitro, using GST-MEK1(K207A) protein as a substrate. Only a small fraction of over-expressed Myc-TPL-2 is associated with p105, presumably due to titration of endogenous p105 protein. The effect of p105 binding on Myc-TPL-2 MEK kinase activity was investigated by incubation of TPL-2 imnmunoprecipitates with recombinant p105₄₉₈₋₉₆₉ protein prior to in vitro kinase assay. MEK phosphorylation was assayed by Western blotting with anti-phospho-MEK-½ antibody. Control experiments confirmed that p105₄₉₈₋₉₆₉ protein bound to TPL-2 in vitro. Addition of p105₄₉₈₋₉₆₉ dramatically inhibited TPL-2 MEK kinase activity in a dose-dependent fashion . 50% inhibition (IC50) was achieved at 0.09 μM p105₄₉₈₋₉₆₉, which represents a molar P105₄₉₈₋₉₆₉/GST-MEK ratio of 0.6: 1. The inhibitory effect of p105₄₉₈₋₉₆₉ was specific since the MEK kinase activity of activated Raf1 (Myc-Raf1(CAAX); (Leevers, S. J., et al (1994) Nature 369: 411-414) was unaffected by addition of p105₄₉₈₋₉₆₉ protein.

(5.6.b) Addition of p105_{498-969(ΔDD}) protein, which lacks the p105 DD, did not affect Myc-TPL-2 MEK kinase activity at the equivalent molar concentrations to p¹⁰⁵₄₉₈₋₉₆₉, suggesting that interaction of the p105 DD with the kinase domain of Myc-TPL-2 inhibited its kinase activity. This hypothesis was tested directly by assaying the effect of addition of recombinant p105₈₀₈₋₈₉₂ protein, which consists solely of the p105 DD. The p105 DD clearly inhibited Myc-TPL-2 MEK kinase activity, although less efficiently than p105₄₉₈₋₉₆₉ protein (TPL-2IC50 (p105DD)=0.55 μM). As expected, the p105 DD had no effect on Myc-Raf1(CAAX) MEK kinase activity.

(5.6.c) The inhibition of TPL-2 MEK kinase activity by p 105₄₉₈₋₉₆₉ may be more efficient than the isolated p105 DD as the former protein contains a binding site for the TPL-2 C-terminus. It was, therefore, interesting to determine the sensitivity of TPL2ΔC catalytic activity to inhibition by p105. The specific activity of Myc-TPL-2ΔC, isolated by immunoprecipitation from transfected 293 cell lysates, was modestly (approximately 2-3 fold) increased relative to wild type Myc-TPL-2. However, Myc-TPL-2ΔC MEK kinase activity was significantly less sensitive to inhibition by p105₄₉₈₋₉₆₉ protein (TPL-2ΔC IC50 (p105₄₉₈₋₉₆₉)=1 μM) than full length Myc-TPL-2 (TPL-2IC50 (p105₄₉₈₋₉₆₉₎=0.09 μM) (FIG. 7 A and B). The isolated p105 DD (p105₈₀₃₋₈₉₃), that lacks the binding site for the TPL-2 C-terminus, inhibited TPL-2ΔC MEK kinase activity with similar efficiency to p105₄₉₈₋₉₆₉ protein, in contrast to its decreased inhibitory effect on wild type Myc-TPL-2. These data suggest that efficient inhibition of TPL-2 MEK kinase activity by the p105 DD requires binding of the TPL-2 C-terminus to p105.

(5.6.d) To determine whether binding to p105 in vivo affects TPL-2 kinase activity, Myc-TPL-2 was transiently expressed in 293 cells in the presence and absence of an excess of wild type HA-p105 and isolated by immunoprecipitation with anti-Myc mAb. Myc-TPL-2 MEK kinase activity was dramatically inhibited by co-expression with HA-p105. However, co-expression with HA-p105_{ΔDD} or HA-p105_{Δ498-541} did not affect Myc-TPL-2 activity. Strikingly, although Myc-TPL-2ΔC clearly associates with HA-p105, its MEK kinase activity was completely unaffected by co-expression with HA-p105.

(5.6.e) In summary, these experiments indicate that the p105 DD inhibits TPL-2 MEK kinase activity and that this inhibitory effect is significantly increased if the TPL-2 C-terminus simultaneously interacts with p105₄₉₈₋₅₄₁. Thus, TPL-2ΔC is less sensitive to negative regulation by p105 in vitro than full length TPL-2 and insensitive to p105 negative regulation when expressed in cells. As a result, the MEK kinase activity of TPLAC is substantially increased (>40-fold) relative to wild type TPL-2 in cells expressing p105.

### EXAMPLE 6

TPL-2 and p105 Protein Expression and Purification

(6.1) TPL-2 and p105 Expression Constructs

Expression constructs for TPL-2 kinase and p105, NF-κB1, were generated by standard molecular biology methods using polymerase chain reaction (PCR) (see Salmeron et al., (2001) J. Biol. Chem. 276: 22215). TPL-2 constructs were expressed using appropriate expression vectors in human HEK 293 cells and/or insect Sf9 cells. Expression of TPL-2 constructs was assessed first by Western analysis on cell lysates and secondly by purification of recombinant TPL-2 protein from cell lysates. Specific examples of constructs generated are outlined in Table I.

TABLE I

| TPL-2 Constructs | Amino acid residues |
|---|---|
| TPL-2 (30-467) | residues 30-467 of SEQ ID 4 |
| TPL-2 (30-397) | residues 30-397 of SEQ ID 4 |
| TPL-2 (104-397) | residues 104-397 of SEQ ID 4 |
| TPL-2 (131-397) | residues 131-397 of SEQ ID 4 |
| TPL-2 (131-411) | residues 131-411 of SEQ ID 4 |
| TPL-2 (1-467) | residues 1-467 of SEQ ID 4 |
| TPL-2 (1-467, M30V) | residues 1-467 of SEQ ID 4 with |
| | substitution of Valine for Methionine at |
| | residue position 30 |
| TPL-2 (30-467, D253N) | residues 30-467 of SEQ ID 4 with |
| | substitution of Asparagine for Aspartic |
| | acid at residue position 253 |
| TPL-2 (30-467, S368A) | residues 30-467 of SEQ ID 4 with |
| | substitution of Alanine for Serine at |
| | residue position 368 |
| TPL-2 (30-467, D253N, S368A) | residues 30-467 of SEQ ID 4 with |
| | substitution of Asparagine for Aspartic |
| | acid at residue position 253 and with |
| | substitution of Alanine for Serine at |
| | residue position 368 |
| TPL-2 (1-467, M30V, D253N) | residues 1-467 of SEQ ID 4 with |
| | substitution of Valine for Methionine at |
| | residue position 30 and with substitution |
| | of Asparagine for Aspartic acid at |
| | residue position 253 |
| TPL-2 (1-467, M30V, D253N, | residues 1-467 of SEQ ID 4 with |
| S368A) | substitution of Valine for Methionine at |
| | residue position 30, with substitution of |
| | Asparagine for Aspartic acid at residue |
| | position 253 and with substitution of |
| | Alanine for Serine at residue position |
| | 368 |
| p105 Constructs | Amino acid residues |
| p105 (369-969) | residues 369-969 of SEQ ID 5 |
| p105 (369-896) | residues 369-869 of SEQ ID 5 |
| p105 (400-896) | residues 400-869 of SEQ ID 5 |
| p105 (444-896) | residues 444-869 of SEQ ID 5 |
| p105 (472-896) | residues 472-869 of SEQ ID 5 |
| p105 (472-896, S894A) | residues 472-896 of SEQ ID 5 and with |
| | substitution of Alanine for Serine at |
| | residue position 894 |
| p105 (1-969, Δ803-893) | residues 1-802 and 894-969 of SEQ |
| | ID 5 with an internal deletion of |
| | residues 803-893 |
| p105 (1-969, Δ498-539) | residues 1-497 and 540-969 of SEQ |
| | ID 5 with an internal deletion of |
| | residues 498-539 |
| p105 (1-969, L842A) | residues 1-969 of SEQ ID 5 with |
| | substitution of Alanine for Leucine at |
| | residue position 842 |
| p105 (498-806), | residues 498-806 of SEQ ID 5 |
| p105 (498-782), | residues 498-782 of SEQ ID 5 |
| p105 (809-893) | residues 809-893 of SEQ ID 5 |

(6.2) TPL-2 and p105 Expression Vectors Containing Epitope Tags

TPL-2 expression vectors containing sequences listed in Table 1 were generated with an N-terminal Flag epitope tag. In addition, TPL-2 expression vectors were also generated with other epitope tags including Myc, GST, His6, and MBP (maltose binding protein). P105 expression vectors containing sequences listed in Table 1 were generated with an N-terminal HA tag. In addition, p105 expression vectors were also generated with other epitope tags including GST or His6. Additionally, TPL-2 and p105 expression vectors were generated with cleavable tags. A protease cleavage site, such as the Tev cleavage site, was introduced between the epitope tag and TPL-2 or epitope tag and p105 sequence respectively. TPL-2 expression vectors were generated with Flag-Tev cleavable tag, and p105 expression vectors were generated with HA-Tev or His6-Tev cleavable tags.

(6.3) Construction of Expression Vectors

(6.3.a) For co-expression in Sf9 cells, TPL-2 and p105 constructs were generated using the baculoviral expression vector, pFastBacdual (Invitrogen). TPL-2 constructs were cloned downstream of the polyhedrin promoter in pFastBacdual, and p105 constructs were cloned downstream of the p10 promoter in pFastBacdual. TPL-2 constructs were also generated using pFastBac1 (Invitrogen) and expressed in Sf9 cells. TPL-2 constructs were cloned downstream of the polyhedrin promoter in pFastBac1.

(6.3.b) For expression in HEK293 cells, TPL-2 and p105 constructs were generated using the expression vector pcDNA3 (Invitrogen). TPL-2 and p105 constructs were co-transfected into HEK293 cells. Transfection into HEK 293 cells was achieved using standard methods such as lipofectamine, calcium phosphate, DEAE dextran, or electroporation (Salmeron et al., (2001) J. Biol. Chem. 276: 22215).

(6.3.c) Examples of Expression Vectors Generated for TPL-2

Examples of preferred TPL-2 expression vectors include but are not limited to the following constructs: COT(30-467) containing an N-terininal Flag epitope tag, COT(30-397) containing an N-terminal Flag epitope tag, COT(104-397) containing an N-terminal Myc epitope tag, COT(131-397) containing an N-terminal Myc epitope tag, COT(131-411) containing an N-terminal Flag epitope tag, COT(1-467) containing an N-terminal Flag epitope tag, COT(1-467, M30V) with an amino acid substitution of valine (V) for methionine (M) at residue 30 and containing an N-terminal Flag epitope tag, COT(30-467, D253N) with an amino acid substitution of asparagine (N) for aspartic acid (D) at residue position 253. COT(30-467, D253N) and containing an N-terminal Flag epitope tag, COT(30-467, S368A) with an amino acid substitution of alanine (A) for serine (D) at residue 368 and containing an N-terminal Flag epitope, COT(30-467, D253N, S368A) with amino acid substitutions of asparagine (N) for aspartic acid (D) at residue 253 and alanine (A) for serine (D) at residue 368 and containing an N-terminal Flag epitope tag, COT(1-467, M30V, D253N) with amino acid substitutions of valine (V) for methionine (M) at residue 30 and asparagine (N) for aspartic acid (D) at residue 253 and containing an N-terminal Flag epitope tag, and COT(1-467, M30V, D253N, S368A) with amino acid substitutions of valine (V) for methionine (M) at residue 30, asparagine (N) for aspartic acid (D) at residue 253 and alanine (A) for serine (D) at residue 368 and containing an N-terminal Flag epitope tag.

(6.3.d) Examples of Expression Vectors Generatedfor p105

Examples of preferred p105 expression vectors include but are not limited to the following constructs: p105(369-969) containing an N-terminal HA epitope tag, p105(369-896) containing an N-terminal HA epitope tag, p105(400-896) containing an N-terminal HA epitope tag, p105(444-896) containing an N-terminal HA epitope tag, p105(472-896) containing an N-terminal HA epitope tag, p105(472-896, S894A) with an amino acid substitution of alanine (A) for serine (S) at residue 894 and containing an N-terminal HA epitope tag, p105(1-969, Δ803-893) with an internal deletion of the death domain of p105 which corresponds to residues 803 to 893 and containing an N-terminal

HA epitope tag, p105(1-969, Δ498-539) with an internal deletion of a stretch of 44 amino acids immediately upstream of the ankyrin repeat region in p105 which corresponds to residues 498 to 541 and containing an N-terminal HA epitope tag, and p105(1-969, L842A) with an amino acid substitution of leucine (L) for alanine (A) at residue 842 and containing an N-terminal HA epitope tag.

(6.4) Baculovirus Generation for TPL2 and TPL2/p105 Expression Vectors

(6.4.a) Transfection of SJ9 Cells with Recombinant Bacmid DNA

5×10⁵ Sf9 cells were seeded per 35 mm well, in 6-well plate, in 2 ml of Sf-900 II SFM. Cells used for transfection were from suspension culture in mid-log phase with a viability of >97%. Cells were allowed to attach at 27° C. for 1 hour. For each transfection, the following solutions were prepared. Solution A: 5 μl of mini-prep bacmid DNA was diluted into 100 μl Sf-900 II SFM. Solution B: 6 μl of CellFECTIN (Invitrogen) reagent was diluted into 100 μl Sf-900 II SFM. Solutions A and B were combined and mixed gently, and incubated for 15 min at room temperature. After incubation, 0.8 ml of Sf-900 II SFM were added to each CellFECTIN-DNA complex and mixed gently. 1 ml of the diluted CellFECTIN-DNA complex was added to the cells in each well without medium. After 5 hour incubation at 27° C., the transfection mixtures were removed and 2 ml of Sf-900 II SFM were added. Cells were incubated at 27° C. for 5 days.

(6.4.b) Harvest of Recombinant Baculovirus and Cell Lysates

Supernatant (2 ml) was collected to a sterile, capped tube and saved at 4° C. 0.5 ml of lysis buffer (50 mM Tris pH 8.0, 137 mM NaCl, 10% Glycerol and 1% Triton×100) was added to each well and incubated for 10 min at room temperature. Lysate was transferred to a 1.5 ml tube and cell debris was removed by spinning the tube for 5 min at 1000 g at room temperature. 10 μl supernatant was separated by 10% SDS-PAGE electrophoresis and protein expression was detected by western blotting with specific antibodies.

(6.4.c) Amplification of Recombinant Baculovirus

2×10⁷ Sf9 cells were seeded in a T150 flask, after attachment, medium was removed and 20 ml of fresh Grace's insect medium plus 10% FBS and 100 μl virus were added to the flask. After incubation for 7 days at 27° C., the supernatant was collected and clarified by centrifugation for 5 min at 1000 g. The amplified virus was kept at 4° C. and protected from light. For long term storage, an aliquot of the viral stock was saved at −80° C.

(6.4.d) Baculovirus Fermentation

Sf-9 insect cells were grown in serum free media (5 L of SF-900 II SFM, Invitrogen) in spinner flasks at 27° C. to a concentration of about 2×10⁶ cells/ml. Cells were infected with baculovirus at MOI of 1-10, then harvested by centrifugation at 48-72 hours.

(6.5) Expression of TPL-2 Kinase in Vitro

(6.5.a) Yield and purity of COT(30-397) and COT(30-467) proteins after expression in Sf9 cells in the presence and absence of p105(369-969) are outlined in Table II.

| | COT | COT(30-397) + | COT | COT(30-467) + |
|---|---|---|---|---|
| | (30-397) | p105(369-969) | (30-467) | p105(369-969) |
| Yield | 0.1 | 0.5 | 0.01 | 1.0 |
| (mg/L) | | | | |
| Purity | 10 | 30 | 1 | 90 |
| (%) | | | | |

Flag-tagged COT(30-397) and COT(30-467) proteins were purified from Sf9 cell lysates using anti-Flag agarose. Purity was assessed by SDS-PAGE and Coomassie blue staining. Total protein recovered was determined from absorbance at 280 nm, then yield of COT(30-397) and COT(30-467) calculated based on purity.

(6.5.b) COT(30-467) containing an N-terminal Flag epitope tag was expressed and purified from Sf9 cells at very low levels, see Table II. COT(30-397) containing an N-terminal Flag epitope tag was also expressed and purified from Sf9 cells at low levels, see Table II.

(6.5.c) COT(104-397) containing an N-terminal Myc epitope tag was expressed in HEK 293 cells at moderate levels. COT(131-397) containing an N-terminal Myc epitope tag was also expressed in HEK 293 cells at moderate levels. COT(131-411) containing an N-terminal Flag epitope tag was expressed in HEK 293 cells at moderate levels. COT(1-467) containing an N-terminal Flag epitope tag was expressed in HEK 293 cells at low levels. COT(30-467, D253N) containing an N-terminal Flag epitope tag was also expressed in HEK 293 cells at low levels. In addition, it was demonstrated that the D253N mutation substantially (≧90%) reduced COT kinase activity as compared to control COT(30-467).

(6.6) Co-expression of COT and p105

(6.6.a) COT and p105 constructs were co-expressed using appropriate expression vectors in human HEK 293 cells and/or insect Sf9 cells. Co-expression of COT and p105 constructs was assessed first by Western analysis on cell lysates, secondly by purification of the COT/p105 protein complex from cell lysates.

(6.6.b) COT(30-467)/p105(369-969) contained COT construct 30-467 and p105 construct 369-969. These COT and p105 constructs were co-expressed at high levels in Sf9 cells, and the COT/p105 complex was purified with good yield and high purity from Sf9 cell lysates, see Table II. Therefore, it was demonstrated that COT(30-467) and p105(369-969) were able to form a stable complex when co-expressed in cells. In addition, the yield of COT(30-467) in complex with p105(369-969) was very substantially higher (≧100-fold) as compared to that obtained for COT(30-467) protein expressed alone in Sf9 cells, see Table II.

(6.6.c) COT(30-397)/p105(369-969) contained COT construct 30-397 and p105 construct 369-969. These COT and p105 constructs were co-expressed at high levels in Sf9 cells, and the COT(30-397)/p105(369-969) complex was purified with good yield and moderate purity from Sf9 cell lysates, see Table II. Therefore, it was demonstrated that COT(30-397) and p105(369-969) were able to form a complex when co-expressed in cells. In addition, the yield of COT(30-397) in complex with p105(369-969) was substantially higher (≧5-fold) as compared to that obtained for COT(30-397) protein expressed alone in Sf9 cells, see Table II.

(6.6.d) COT(30-467, D253N)/p105(369-896) contained COT construct 30-467, D253N and p105 construct 369-896. These COT and p105 constructs were co-expressed at high levels in Sf9 cells, and the COT/p105 complex was purified with good yield and high purity from Sf9 cell lysates.

(6.6.e) COT(30-467, D253N)/p105(472-896) contained COT construct 30-467, D253N and p105 construct 472-896. These COT and p105 constructs were co-expressed at high levels in Sf9 cells, and the COT/p105 complex was purified with good yield and high purity from Sf9 cell lysates.

(6.6.f) COT(30-467, D253N, S368A)/p105(472-896, S894A) contains COT construct 30-467, D253N, S368A and p105 construct 472-896, S894A. These COT and p105 constructs are co-expressed in Sf9 cells, and the COT/p105 complex is purified from Sf9 cell lysates.

(6.6.g) COT(30-467)/p105(400-896) contained COT construct 30-467 and p105 construct 400-896. These COT and p105 constructs were co-expressed at moderate levels in HEK293 cells, and the COT/p105 complex was isolated from HEK293 cell lysates.

(6.6.h) COT(30-467)/p105(444-896) contained COT construct 30-467 and p105 construct 444-896. These COT and p105 constructs were co-expressed at moderate levels in HEK293 cells, and the COT/p105 complex was isolated from HEK293 cell lysates.

(6.6.i) COT(131-41 1)/p105(369-969) contained COT construct 131-411 and p105 construct 369-969. These COT and p105 constructs were co-expressed at moderate levels in HEK293 cells, and the COT/p105 complex was isolated from HEK293 cell lysates.

(6.6.j) COT(1-467)/p105(369-969) contained COT construct 1-467 and p105 construct 369-969. These COT and p105 constructs were co-expressed at moderate levels in Sf9 cells, and the COT/p105 complex was purified with moderate yield and low purity from Sf9 cell lysates.

(6.6.k) COT(1-467, M30V, D253N)/p105(369-896) contained COT construct 1-467, M30 V, D253N and p105 construct 369-896. These COT and p105 constructs were co-expressed at moderate levels in Sf9 cells, and the COT/p105 complex was purified with moderate yield and low purity from Sf9 cell lysates.

(6.7) COT and COT/p105 Protein Purification

(6.7.a) COT(30-467)/p105(369-969)

Cell pellet from 1 L fermentation of Sf9 cells expressing Flag-COT(30-467) and HA-p105(369-969), by baculovirus infection was lysed in 50 ml of Triton lysis buffer (1% Triton X-100, 20 mM Tris, pH 8.0, 150 mM NaCl, 10% glycerol). Lysate was centrifuged at 17,000 rpm for 30 min at 4° C. in Sorvall SS-34 rotor. Supernatant was applied at a flow rate of 0.2 ml/min to a 1 ml column of anti-Flag m2 agarose (Sigma) which was equilibrated in 50 mM HEPES, pH 7.5, 0.2 M NaCl, 2 mM DTT. Protein was eluted with 0.3 mg/ml Flag peptide dissolved in the equilibration buffer. Column fractions were analyzed by SDS-PAGE and Coomassie blue staining. A total of 1.0 mg of COT(30-467)/p105(369-969) protein was recovered with an approximate purity of 90% (Table II).

The purified COT(30-467)/p105(369-969) was then chromatographed on a Superose 6 column equilibrated in 50 mM HEPES pH 7.5, 0.2 M NaCl, 2 mM DTT. This column is capable of separating proteins by their size and is useful in analyzing protein complexes and aggregates. Chromatography of COT(30-467)/p105(369-969) resulted in two peaks as detected by A₂₈₀, that when run on SDS-PAGE showed that both COT and p105 eluted to together on this column in two pools. Approximately 25% of the complex was in the excluded volume (Vo) indicating a MW of greater than 4×10⁴ kDa. The second peak, containing 75% of the COT(30-467)/p105(369-969), eluted with an apparent MW of 600 kDa. Most (>80%) of the COT kinase activity as measured by a COT cascade HTRF assay was in the second peak.

(6.7.b) COT(30-467)

Cell pellet from 1 L fermentation of Sf9 cells expressing Flag-COT(30-467) was purified following the protocol used above for COT-467/p105ΔN. The amount of recovered COT(30-467) protein was too low to accurately quantitate (≦10 ug/L, Table II). Western blot analysis was necessary to confirm that a minor band detected following SDS-PAGE and Coomassie Blue staining was COT(30-467) protein.

(6.7.c) COT(30-397)

Cell pellet from 1 L fermentation of Sf9 cells expressing Flag-COT(30-397) by baculovirus infection was lysed in 50 ml of Triton lysis buffer (N% Triton X-100, 20 mM Tris pH 8.0, 150 mM NaCl, 10% glycerol). Lysate was centrifuged at 17,000 rpm for 30 min. at 4° C. in Sorvall SS-34 rotor. Supernatant was applied at a flow rate of 0.2 ml/min. to a 1 ml column of anti-Flag m2 agarose (Sigma) which was equilibrated in 50 mM HEPES, pH 7.5, 0.2 M NaCl, 2 mM DTT. Protein was eluted with 0.3 mg/ml Flag peptide dissolved in the equilibration buffer. Column fractions were analyzed by SDS-PAGE and Coomassie blue staining. The result obtained was multiple protein bands, only one of which at approximately 45 kDa was confirmed by Western blotting to be COT(30-397). The COT(30-397) protein was about 10% pure and approximately 0.1 mg was recovered from 1 L of Sf9 cells (Table II).

When COT(30-397) was chromatographed on a Superose 6 column there were two peaks as detected by A₂₈₀. The excluded column volume consisted of about 90% of the COT(30-397) protein along with non-COT impurities. The second peak at about 45 kDa was mainly COT(30-397) protein. All of the COT kinase activity as measured by a COT cascade HTRF assay was in the 45 kDa peak.

(6.7.d) COT(30-397)/p105(369-969)

Cell pellet from 1 L fermentation of Sf9 cells expressing COT(30-397)/p105(369-969), by baculovirus infection was lysed in 50 ml of Triton lysis buffer (1% Triton X-100, 20 mM Tris, pH 8.0, 150 mM NaCl, 10% glycerol). Lysate was centrifuged at 17,000 rpm for 30 min. at 4° C. in Sorvall SS-34 rotor. Supernatant was applied at a flow rate of 0.2 ml/min to a 1 ml column of anti-Flag m2 agarose (Sigma) which was equilibrated in 50 mM HEPES pH 7.5, 0.2 M NaCl, 2 mM DTT. Protein was eluted with 0.3 mg/ml Flag peptide dissolved in the equilibration buffer. Column fractions were analyzed by SDS-PAGE and Coomassie blue staining and at least three major bands were detected. Two of the prominent protein bands with apparent MW's of about 45 KDa and about 70 kDa were confirmed to be COT(30-397) and p105(369-969) respectively, by Western blotting and MS analysis. A total of 0.5 mg of COT(30-397)/p105(369-969) protein was recovered with an approximate purity of 30% (Table II).

The purified COT(30-397)/p105(369-969) was then chromatographed on a Superose 6 column equilibrated in 50 mM HEPES pH 7.5, 0.2 M NaCl, 2 mM DTT. This resulted in two major peaks as detected by A₂₈₀, one in the excluded volume and the second at about 45 kDa. Analysis of column fractions on SDS-PAGE showed that the first peak (Vo) contained both COT(30-397) and p105(369-969), whereas the peak at 45 kDa contained COT(30-397) protein without any associated p105(369-969). This indicates that the COT(30-397)/ p105(369-969) complex is dissociating during chromatography on the Superose 6 column. Most (>80%) of the COT kinase activity as measured by a COT cascade HTRF assay was in the 45 kDa peak.

(6.8) Dissociation of COT(30-467)/p105(369-969) Complex Using 8 M Urea

Urea was added to a sample of COT(30-467)/p105(369-969), purified as in (6.7.a), to a final concentration of 8 M. Sample was chromatographed on a Superose 6 column equilibrated in 8 M urea, 50 mM HEPES pH 7.5, 0.2 M NaCl. The resulting two peaks detected by A₂₈₀ were then analyzed by SDS-PAGE and Coommassie blue staining. The first peak contained one protein band with MW about 70 kDa, corresponding to p105(369-969), while the second peak contained predominantly the 50 kDa, corresponding to COT(30-467). The identity of the 70 kDA and 50 kDa proteins was confirmed by Western blotting. The same experiment using 2 M or 4 M urea failed to separate the COT(30-467)/p105(369-969) complex. In addition, in other experiments using increasing salt concentrations up to and including 1 M NaCl failed to separate the COT(30-467)/p105(369-969) complex.

(6.9) Phosphorylation Site Mapping on COT(30-467, D253N)/p105(369-896)

COT(30-467, D253N)/p105(369-896) protein complex was purified from Sf9 cell pellets as described in (6.7.a). The purified COT/p105 complex was digested with trypsin by standard methods. Tryptic peptides derived from COT(30-467, D253N) and p105(369-896) were separated by HPLC and phosphorylation state was analyzed by mass spectroscopy. This method identified one major phosphorylation site in COT and one major site in p105. The phosphorylation site found in COT(30-467, D253N) was the serine residue 368 (S368), see sequence ID 4. The phosphorylation site found in p105(369-969) was the serine residue 894 (S894), see sequence ID 5.

Equivalents

Those skilled in the art will recognize, or be able to ascertain using no more than routine experimentation, many equivalents to the specific embodiments of the invention described herein. Such equivalents are intended to be encompassed by the following claims.

What is claimed:

1. A method for modulating NFκB activity comprising,
contacting a TPL-2 molecule with a component of NFκB regulation such that modulation of NFκB activity occurs.

2. The method according to claim 1, wherein the TPL-2 molecule is wild-type TPL-2.

3. The method according to claim 1, wherein the TPL-2 molecule retains the p105-phosphorylating activity of wild-type TPL-2.

4. The method according to claim 1, wherein the TPL-2 molecule is a dominant negative TPL-2 mutant.

5. The method according to claim 1, wherein the TPL-2 molecule retains the C-terminus of wild-type TPL-2.

6. A method for identifying a compound or compounds capable, directly or indirectly, of modulating the activity of p105, comprising the steps of:
(a) incubating a TPL-2 molecule with the compound or compounds to be assessed; and
(b) identifying those compounds which influence the activity of the TPL-2 molecule.

7. A method according to claim 6, wherein the compound or compounds bind to the TPL-2 molecule.

8. A method according to claim 6 or claim 7, further comprising
(c) assessing the compounds which influence the activity of TPL-2 for the ability to modulate NFκB activation in a cell-based assay.

9. A method for identifying a lead compound for a pharmaceutical useful in the treatment of disease involving or using an inflammatory response, comprising:
incubating a compound or compounds to be tested with a TPL-2 molecule and p105, under conditions in which, but for the presence of the compound or compounds to be tested, TPL-2 associates with p105 with a reference affinity;
determining the binding affinity of TPL-2 for p105 in the presence of the compound or compounds to be tested; and
selecting those compounds which modulate the binding affinity of TPL-2 for p105 with respect to the reference binding affinity.

10. A method for identifying a lead compound for a pharmaceutical useful in the treatment of disease involving or using an inflammatory response, comprising:
incubating a compound or compounds to be tested with a TPL-2 molecule and p105, under conditions in which, but for the presence of the compound or compounds to be tested, TPL-2 associates with p105 with a reference affinity;
determining the binding affinity of TPL-2 for p105 in the presence of the compound or compounds to be tested; and
selecting those compounds which modulate the binding affinity of TPL-2 for NFκB with respect to the reference binding affinity.

11. A method for identifying a lead compound for a pharmaceutical, comprising:
incubating a compound or compounds to be tested with a TPL-2 molecule and tumour necrosis factor (TNF), under conditions in which, but for the presence of the compound or compounds to be tested, the interaction of TNF and TPL-2 induces a measurable chemical or biological effect;
determining the ability of TNF to interact, directly or indirectly, with TPL-2 to induce the measurable chemical or biological effect in the presence of the compound or compounds to be tested; and
selecting those compounds which modulate the interaction of TNF and TPL-2.

12. A method according to claim 11, which is carried out in vivo in a cell.

13. A method for identifying a lead compound for a pharmaceutical, comprising the steps of:
providing a purified TPL-2 molecule;
incubating the TPL-2 molecule with a substrate known to be phosphorylated by TPL-2 and a test compound or compounds; and
identifying the test compound or compounds capable of modulating the phosphorylation of the substrate.

14. A method according to claim 13, wherein the substrate is MEK.

15. A compound identifiable by the method of any one of claims 6 to 14, capable of modulating the direct or indirect interaction of TPL-2 with p105.

16. A compound according to claim 15, which is an antibody.

17. An antibody according to claim 16, which is specific for TPL-2.

18. A compound according to claim 15, which is a polypeptide.

19. A polypeptide according to claim 18, which is a TPL-2 molecule.

20. A polypeptide according to claim 19, which is a constitutively active mutant or a dominant negative mutant of TPL-2.

21. A method for modulating the activity of p105 in a cell, comprising administering to the cell a compound according to any one of claims 15 to 20.

22. A pharmaceutical composition comprising, as active ingredient, a therapeutically effective amount of a compound according to any one of claims 15 to 20.

23. Use of a compound according to any one of claims 15 to 20 for the treatment of a condition associated with NFκB induction or repression.

24. A method for treating a condition associated with NFκB induction or repression, comprising administering to a subject a therapeutically effective amount of a compound according to any one of claims 15 to 20.

25. A method for identifying a compound which regulates an inflammatory response mediated by TPL-2 comprising,
contacting a reaction mixture that comprises a TPL-2 polypeptide, or fragment thereof, with a test compound; and
determining the effect of the test compound on an indicator of NFκB activity to thereby identify a compound that regulates NFκB activity mediated by TPL-2.

26. A method for identifying a compound which regulates NFκB activity mediated by TPL-2 comprising,
contacting a reaction mixture that comprises a TPL-2 polypeptide, or fragment thereof, with a test compound; and
determining the effect of the test compound on an indicator of NFκB activity to thereby identify a compound that regulates NFκB activity mediated by TPL-2.

27. A method for identifying a compound which regulates signal transduction by TPL-2 comprising,
contacting a reaction mixture that comprises a TPL-2 polypeptide, or a fragment thereof, with a test compound, and
determining the effect of the test compound on an indicator of signal transduction by the TPL-2 polypeptide in the reaction mixture to thereby identify a compound which regulates signal transduction by TPL-2.

28. A method for identifying a compound which modulates the interaction of a TPL-2 polypeptide with a target component of TPL-2 modulation comprising,
contacting a reaction mixture that comprises a TPL-2 polypeptide or fragment thereof, with a target component of said TPL-2 modulation, and
a test compound, under conditions whereby, but for the presence of said test compound, said TPL-2 polypeptide, or fragment thereof, specifically interacts with said target component at a reference level and determining a change in the level of interaction in the presence of the test compound, wherein a difference indicates that said test compound modulates the interaction of a TPL-2 polypeptide, or fragment thereof, with a target component of TPL-2 modulation.

29. The method according to any one of claims 25, 26, 27, and 28, wherein the TPL-2 polypeptide comprises an amino acid sequence having at least 75% identity with a polypeptide selected from the group consisting of SEQ ID NO:2 and 4.

30. The method according to any one of claims 25, 26, 27, and 28, wherein the TPL-2 polypeptide is encoded by a nucleic acid molecule which hybridizes under highly stringent conditions with a nucleic acid molecule selected from the group consisting of SEQ ID NO:1 and 3.

31. The method according to any one of claims 25, 26, 27, and 28, wherein the reaction mixture is a cell-free mixture.

32. The method according to any one of claims 25, 26, 27, and 28, wherein the reaction mixture is a cell-based mixture.

33. The method according to claim 32, wherein the reaction mixture is a recombinant cell.

34. The method according to claim 33, wherein said recombinant cell comprises a heterologous nucleic acid encoding a TPL-2 polypeptide.

35. The method according to any one of claims 25, 26, 27, and 28, wherein said determining comprises measuring a TPL-2 activity selected from the group consisting of, kinase activity, binding activity, and signaling activity.

36. The method according to claim 35, wherein said TPL-2 activity is kinase activity

37. The method according to any one of claims 25, 26, 27, and 28, wherein the recombinant cell includes a reporter gene construct comprising a reporter gene in operable linkage with a transcriptional regulatory sequence sensitive to intracellular signals transduced by TPL-2 or NFκB.

38. The method according to claim 37, wherein said transcriptional regulatory sequence comprises a TNF transcriptional regulatory sequence.

39. The method according to claim 28, wherein said target component is selected from the group consisting of, p105, IκB-α, IκB-β, MEK-1, SEK-1, and NFκB.

40. The method according to any one of claims 25, 26, 27, and 28, wherein said TPL-2 molecule is a recombinant polypeptide.

41. The method according to claim 40, wherein said TPL-2 polypeptide comprises an amino acid sequence selected from the group consisting of SEQ If) NO: 2 and 4.

42. The method according to claim 35, wherein said signaling comprises TNF expression.

43. The method according to claim 37, wherein said recombinant cell comprises a reporter gene sensitive to TPL-2 signal transduction.

44. The method according to any one of claims 25, 26, 27, and 28, wherein said determining comprises measuring apoptosis of a cell.

45. The method according to any one of claims 25, 26, 27, and 28, wherein said determining comprises measuring cell proliferation.

46. The method according to any one of claims 25, 26, 27, and 28, wherein said determining comprises measuring an immune response.

47. The method according to any one of claims 25, 26, 27, and 28, wherein the TPL-2 polypeptide is a purified TPL-2 polypeptide.

48. The method according to claim 28, wherein said target component is provided as a purified polypeptide.

49. The method according to claim 28, wherein said target component is a polypeptide, or fragment thereof, selected from the list comprising p105, IκB-α, IκB-β, MEK-1, SEK-1, and NFκB.

50. The method according to claim 49, wherein said target component is IκB-α.

51. The method according to claim 49, wherein said target component is p105.

52. The method according to any one of claims 25, 26, 27, and 28, wherein said test compound is selected from the group consisting of protein based, carbohydrate based, lipid based, nucleic acid based, natural organic based, synthetically derived organic based, and antibody based compounds.

53. A compound identified according to the method of any one of claims 25, 26, 27, and 28.

54. A compound identified according to the method of any one of claims 25, 26, 27, and 28, wherein said compound is suitable for treating a condition selected from the group consisting of rheumatoid arthritis, multiple sclerosis (MS), inflammatory bowel disease (IBD), insulin-dependent diabetes mellitus (IDDM), sepsis, psoriasis, misregulated TNF expression, and graft rejection.

55. A compound identified according to the method of any one of claims 25, 26, 27, and 28, wherein said compound is suitable for treating rheumatoid arthritis.

56. A compound identified according to the method of any one of claims 25, 26, 27, and 28, wherein said compound is suitable for treating misregulated TNF expression.

57. A method for treating an immune system condition in a subject in need thereof by modulating TPL-2 activity comprising,
administration of a pharmaceutical composition able to modulate TPL-2, said administration in an amount sufficient to modulate the immune system response in said patient.

58. A method for treating a TPL-2-mediated condition in a subject comprising,
administering composition capable of modulating TPL-2 in a therapeutically effective amount sufficient to modulate said TPL-2-mediated condition in said subject.

59. A method for modulating TPL-2-mediated NFκB regulation in a subject in need thereof comprising,
administering a therapeutically-effective amount of a pharmaceutical composition to the human such that modulation occurs.

60. A method for modulating TPL-2-mediated NFκB regulation within a cell comprising,
administering to a cell a composition capable of modulating TPL-2 in an amount sufficient such that a change in TPL-2-mediated NFκB regulation is achieved.

61. The method of according to any one of claims 57 and 58, wherein said condition is elected from the group consisting of rheumatoid arthritis, multiple sclerosis (MS), inflammatory bowel disease (IBD), insulin-dependent diabetes mellitus (IDDM), sepsis, psoriasis, misregulated TNF expression, and graft rejection.

62. The method of claim 61, wherein said condition is rheumatoid arthritis.

63. The method of claim 61, wherein said condition misregulated TNF expression.

64. The method according to any one of claim 57-59, wherein said composition is selected from the group consisting of N-(6-phenoxy-4-quinolyl)-N-[4-(phenylsulfanyl)phenyl]amine], ethyl 5-oxo-4-[4-(phenylsulfanyl)anilino]-5,6,7,8-tetrahydro-3-quinolinecarboxylate, 3-(4-pyridyl)-4,5-dihydro-2H-benzo[g]indazole methanesulfonate, and sodium 2-chlorobenzo[1][1,9]phenanthroline-7-carboxylate.

65. A method for treating TNF misregulation comprising,
administering to a subject at risk for TNF nisregulation a therapeutically effective amount of a TPL-2 modulator such that treatment occurs.

66. The method of claim 65, wherein said TPL-2 modulator is selected from the group consisting of N-(6-phenoxy-4-quinolyl)-N-[4-(phenylsulfanyl)phenyl]amine], ethyl 5-oxo-4-[4-(phenylsulfanyl)anilino]-5,6,7,8-tetrahydro-3-quinolinecarboxylate, 3-(4-pyridyl)-4,5-dihydro-2H-benzo[g]indazole methanesulfonate, and sodium 2-chlorobenzo[1][1,9]phenanthroline-7-carboxylate.

67. A method for treating rheumatoid arthritis comprising,
administering to a subject at risk for rheumatoid arthritis a therapeutically effective amount of a TPL-2 modulator such that treatment occurs.

68. The method of claim 67, wherein said TPL-2 modulator is selected from the group consisting of N-(6-phenoxy-4-quinolyl)-N-[4-(phenylsulfanyl)phenyl]amine], ethyl 5-oxo-4-[4-(phenylsulfanyl)anilino]-5,6,7,8-tetrahydro-3-quinolinecarboxylate, , 3-(4-pyridyl)-4,5-dihydro-2H-benzo[g]indazole methanesulfonate, and sodium 2-chlorobenzo[1][1,9]phenanthroline-7-carboxylate.

69. A method for identifying a compound capable of modulating the interaction of a TPL-2 polypeptide or functional fragment thereof with a target component of TPL-2 modulation, wherein said TPL-2 interacts with said target component at a reference level of interaction, the method comprising:
(a) providing a test compound;
(b) contacting a TPL-2 polypeptide or functional fragment thereof, and a target component of TPL-2 modulation with said test compound, in a reaction mixture under sufficient conditions and for a sufficient time to permit interaction of said TPL-2 polypeptide or fragment thereof with said target component; and
(c) detecting a change in the level of interaction between said TPL-2 or functional fragment thereof and said target component compared to said reference level of interaction.

70. The method according to claim 69, wherein said target component is selected from the group consisting of; p105, IκB-α, IκB-β, MEK-1, SEK-1, and NFκB, or a functional fragment thereof.

71. The method of claim 70 wherein measurement of said level of interaction is determined by a method selected from the group consisting of: measuring TPL-2 kinase activity, measuring TPL-2 binding activity, measuring and TPL-2 signal transduction activity.

72. The method of claim 70 wherein measurement of said level of interaction is determined by a method selected from the group consisting of: measuring the binding affinity of TPL-2 for p105, measuring p105 phosphorylation, measuring p105 degradation, measuring IκB-α activity, measuring IκB-β activity, measuring MEK-1 phosphorylation, measuring MEK-1 activity, measuring SEK-1 activity, measuring NFκB activity, and measuring TNF activity.

73. The method of claim 70, wherein said reaction mixture is a cell-free mixture.

74. The method of claim 70, wherein said reaction mixture is a cell-based mixture.

75. The method according to claim 74, wherein the reaction mixture is a recombinant cell.

76. The method according to claim 75, wherein said recombinant cell comprises a heterologous nucleic acid encoding a TPL-2 polypeptide or functional fragment thereof.

77. The method of claim 70 wherein measurement of said level of interaction is determined by a method selected from the group consisting of; measuring TNF expression, measuring apoptosis of cells, measuring cell proliferation, measuring an immune response, and measuring an inflammatory response.

78. The method according to claim 69, wherein said test compound is selected from the group consisting of; protein based, carbohydrate based, lipid based, nucleic acid based, natural organic based, synthetically derived organic based, and antibody based compounds.

79. The method according to claim 69, wherein said TPL-2 polypeptide or a functional fragment thereof is selected from the group consisting of; wild-type TPL-2 or functional fragment thereof, a purified TPL-2 polypeptide or functional fragment thereof, a recombinant TPL-2 polypeptide or functional fragment thereof, a dominant negative TPL-2 mutant or functional fragment thereof, a polypeptide comprising an amino acid sequence having at least the C-terminus of TPL-2 (residues 398-467 of SEQ ID NO:4), a polypeptide comprising an amino acid sequence having at least 75% identity to SEQ ID NO:2, a polypeptide comprising an amino acid having at least 75% identity to sequence of SEQ ID NO:4, TPL-2 (30-467), TPL-2 (30-397), TPL-2 (104-397), TPL-2 (131-397), TPL-2 (131-411), TPL-2 (1-467), TPL-2 (1-467, M30V), TPL-2 (30- 467, D253N), TPL-2 (30-467, S368A), TPL-2 (30-467, D253N, S368A), TPL-2 (1-467, M30V, D253N), and TPL-2 (1-467, M30V, D253N, S368A).

80. The method according to claim 69, wherein said target component is p105 or a functional fragment thereof.

81. The method according to claim 69, wherein said target component is a polypeptide selected from the group consisting of; a polypeptide comprising an amino acid sequence having at least at least 75% identity to the C-terminus of p105 (residues 498-969 of SEQ ID NO: 5), a polypeptide comprising an amino acid sequence having at least 75% identity to residues 498-541 of SEQ ID NO:5), and a polypeptide comprising an amino acid sequence having at least 75% identity to residues 803-893 of SEQ ID NO:5, p105 (369-969), p105 (369-896), p105 (400-896), p105 (444-896), p105 (472-896), p105 (472-896, S894A), p105 (1-969, Δ803-893), p105 (1-969, Δ498-541), p105 (1-969, L842A), p105(498-806), p105(498-782), and p105(809-893).

82. A compound identified according to the method of any one of claims 69-81.

83. The compound of claim 82, wherein said compound is a polypeptide.

84. The compound of claim 83, wherein said compound is selected from the group consisting of; wild-type TPL-2 or functional fragment thereof, a purified TPL-2 polypeptide or functional fragment thereof, a recombinant TPL-2 polypeptide or functional fragment thereof, a dominant negative TPL-2 mutant or functional fragment thereof, a polypeptide comprising an amino acid sequence having at least the C-terminus of TPL-2 (residues 398-467 of SEQ ID NO:4), a polypeptide comprising an amino acid sequence having at least 75% identity to SEQ ID NO:2, a polypeptide comprising an amino acid having at least 75% identity to sequence of SEQ ID NO:4, TPL-2 (30-467), TPL-2 (30-397), TPL-2 (104-397), TPL-2 (131-397), TPL-2 (131-411), TPL-2 (1-467), TPL-2 (1-467, M30V), TPL-2 (30-467, D253N), TPL-2 (30-467, S368A), TPL-2 (30-467, D253N, S368A), TPL-2 (1-467, M30V, D253N), and TPL-2 (1-467, M30V, D253N, S368A).

85. The compound of claim 83, wherein said compound is a p105 molecule or functional fragment thereof.

86. The compound of claim 83, wherein said compound is a polypeptide selected from the group consisting of; a polypeptide comprising an amino acid sequence having at least at least 75% identity to the C-terminus of p105 (residues 498-969 of SEQ ID NO: 5), a polypeptide comprising an amino acid sequence having at least 75% identity to residues 498-541 of SEQ ID NO:5), and a polypeptide comprising an amino acid sequence having at least 75% identity to residues 803-893 of SEQ ID NO:5, p105 (369-969), p105 (369-896), p105 (400-896), p105 (444-896), p105 (472-896), p105 (472-896, S894A), p105 (1-969, Δ803-893), p105 (1-969, Δ498-541), p105 (1-969, L842A), p105(498-806), p105(498-782), and p105(809-893).

87. A pharmaceutical composition comprising a therapeutically effective amount of a compound according to any one of claims 82-86 and a pharmaceutically acceptable carrier.

88. A method for modulating the interaction between TPL-2 and a target component of TPL-2 modulation in a cell, comprising the steps of:
(a) providing a cell, wherein TPL-2 interacts with a target component of TPL-2 modulation; and
(b) administering to said cell a compound capable of modulating the interaction between TPL-2 and said target component in an amount effective to modulate the interaction between TPL-2 and said target component.

89. The method according to claim 88, wherein said target component is selected from the group consisting of; p105, IκB-α, IκB-β, MEK-1, SEK-1, and NFκB.

90. The method according to claim 89, wherein said compound capable of modulating the interaction between TPL-2 and said target component is selected from the group consisting of; wild-type TPL-2 or functional fragment thereof a purified TPL-2 polypeptide or functional fragment thereof, a recombinant TPL-2 polypeptide or functional fragment thereof, a dominant negative TPL-2 mutant or functional fragment thereof, a polypeptide comprising an amino acid sequence having at least the C-terminus of TPL-2 (residues 398-467 of SEQ ID NO:4), a polypeptide comprising an amino acid sequence having at least 75% identity to SEQ ID NO:2, a polypeptide comprising an amino acid having at least 75% identity to sequence of SEQ ID NO:4, TPL-2 (30-467), TPL-2 (30-397), TPL-2 (104-397), TPL-2 (131-397), TPL-2 (131-411), TPL-2 (1-467), TPL-2 (1-467, M30V), TPL-2 (30-467, D253N), TPL-2 (30-467, S368A), TPL-2 (30-467, D253N, S368A), TPL-2 (1-467, M30V, D253N), and TPL-2 (1-467, M30V, D253N, S368A).

91. The method according to claim 89, wherein said compound capable of modulating the interaction between TPL-2 and said target component is a polypeptide selected from the group consisting of; a polypeptide comprising an amino acid sequence having at least at least 75% identity to the C-terminus of p105 (residues 498-969 of SEQ ID NO: 5), a polypeptide comprising an amino acid sequence having at least 75% identity to residues 498-541 of SEQ ID NO:5), and a polypeptide comprising an amino acid sequence having at least 75% identity to residues 803-893 of SEQ ID NO:5, p105 (369-969), p105 (369-896), p105 (400-896), p105 (444-896), p105 (472-896), p105 (472-896, S894A), p105 (1-969, Δ803-893), p105 (1-969, Δ498-541), p105 (1-969, L842A), p105(498-806), p105(498-782), and p105(809-893).

92. The method according to claim 89, wherein said compound capable of modulating the interaction between TPL-2 and said target component is a compound according to any one of claims 82-87.

93. The method according to claim 89, wherein said compound capable of modulating the interaction between TPL-2 and said target component is selected from the group consisting of; N-(6-phenoxy-4-quinolyl)-N-[4-(phenylsulfanyl)phenyl]amine]ethyl 5-oxo-4-[4-(phenylsulfanyl)anilino]-5,6,7,8-tetrahydro-3-quinolinecarboxylate, and sodium 2-chlorobenzo[1][1,9]phenanthroline-7-carboxylate.

94. A method for treating or slowing the progression of a disease or disorder mediated by the interaction between TPL-2 and a target component of TPL-2 modulation in a cell, the method comprising;
(a) providing an individual afflicted with a disease or disorder mediated by the interaction between TPL-2 and a target component of TPL-2 modulation; and
(b) administering to said individual a therapeutically effective amount of a compound capable of modulating the interaction between TPL-2 and said target component.

95. The method according to claim 94, wherein said target component is selected from the group consisting of; p105, IκB-α, IκB-β, MEK-1, SEK-1, and NFκB, or a functional fragment thereof.

96. The method according to claim 94, wherein said disease or disorder mediated by the interaction between TPL-2 and said target component is an undesirable immune system response.

97. The method according to claim 94, wherein said disease or disorder mediated by the interaction between TPL-2 and said target component is selected from the group consisting of; rheumatoid arthritis, multiple sclerosis (MS), inflammatory bowel disease (IBD), insulin-dependent diabetes mellitus (IDDM), sepsis, psoriasis, misregulated TNF expression, cancer, and graft rejection.

98. The method according to claim 95, wherein said compound capable of modulating the interaction between TPL-2 and said target component is selected from the group consisting of; wild-type TPL-2 or functional fragment thereof, a purified TPL-2 polypeptide or functional fragment thereof, a recombinant TPL-2 polypeptide or functional fragment thereof, a dominant negative TPL-2 mutant or functional fragment thereof, a polypeptide comprising an amino acid sequence having at least the C-terminus of TPL-2 (residues 398-467 of SEQ ID NO:4), a polypeptide comprising an amino acid sequence having at least 75% identity to SEQ ID NO:2, a polypeptide comprising an amino acid having at least 75% identity to sequence of SEQ ID NO:4, TPL-2 (30-467), TPL-2 (30-397), TPL-2 (104-397), TPL-2 (131-397), TPL-2 (131-411), TPL-2 (1-467), TPL-2 (1-467, M30V), TPL-2 (30-467, D253N), TPL-2 (30-467, S368A), TPL-2 (30-467, D253N, S368A), TPL-2 (1-467, M30V, D253N), and TPL-2 (1-467, M30V, D253N,S368A).

99. The method according to claim 95, wherein said compound capable of modulating the interaction between TPL-2 and said target component is a polypeptide selected from the group consisting of; a polypeptide comprising an amino acid sequence having at least at least 75% identity to the C-terminus of p105 (residues 498-969 of SEQ ID NO: 5), a polypeptide comprising an amino acid sequence having at least 75% identity to residues 498-541 of SEQ ID NO:5), and a polypeptide comprising an amino acid sequence having at least 75% identity to residues 803-893 of SEQ ID NO:5, p105 (369-969), p105 (369-896), p105 (400-896), p105 (444-896), p105 (472-896), p105 (472-896, S894A), p105 (1-969, Δ803-893), p105 (1-969, Δ498-541), p105 (1-969, L842A), p105(498-806), p105(498-782), and p105(809-893).

100. The method according to claim 95, wherein said compound capable of modulating the interaction between TPL-2 and said target component is a compound according to any one of claims 82-87.

101. The method according to claim 95, wherein said compound capable of modulating the interaction between TPL-2 and said target component is selected from the group consisting of; N-(6-phenoxy-4-quinolyl)-N-[4-(phenylsulfanyl)phenyl]amine], ethyl 5-oxo-4-[4-(phenylsulfanyl)anilino]-5,6,7,8-tetrahydro-3-quinolinecarboxylate, and sodium 2-chlorobenzo[1][1,9]phenanthroline-7-carboxylate.

102. A composition of matter comprising:
a first nucleic acid comprising a first nucleic acid sequence encoding a first polypeptide; and
a second nucleic acid comprising a second nucleic acid sequence encoding a second polypeptide, wherein said first polypeptide comprises TPL-2 kinase, and
said second polypeptide comprises p105.

103. The composition of matter according to claim 102, further comprising:
a first expression vector; and
a second expression vector,
wherein said first expression vector comprises said first nucleic acid and said second expression vector comprises said second nucleic acid.

104. The composition of matter according to claim 102, further comprising an expression vector, wherein said expression vector comprises said first nucleic acid and said second nucleic acid.

105. The composition of matter according to claim 104, wherein said first nucleic acid is operatively linked to a first promoter and said second nucleic acid is operatively linked to a second promoter.

106. The composition of matter according to claim 104, wherein said first nucleic acid and said second nucleic acid are operably linked to one promoter.

107. The composition of matter according to claims 103 or 104, wherein said first nucleic acid and said second nucleic acid are operably linked to promoters, said promoters independently selected from the group consisting of retroviral promoters, viral promoters, bacterial promoters, baculoviral promoters, and eukaryotic promoters.

108. The composition of matter according to claim 107, wherein said baculoviral promoters are independently selected from the group consisting of a polyhedrin promoter and a p10 promoter.

109. The composition of matter according to claim 102 wherein said TPL-2 kinase is selected from the group consisting of TPL-2 (30-467), TPL-2 (30-397), TPL-2 (104-397), TPL-2 (131-397), TPL-2 (131-411), TPL-2 (1-467), TPL-2 (1-467, M30V), TPL-2 (30-467, D253N), TPL-2 (30-467, S368A), TPL-2 (30-467, D253N, S368A), TPL-2 (1-467, M30V, D253N), and TPL-2 (1-467, M30V, D253N, S368A).

110. The composition of matter according to claim 102, wherein said TPL-2 kinase comprises TPL-2(30-467).

111. The composition of matter according to claim 102, wherein said TPL-2 kinase comprises TPL-2(30-397).

112. The composition of matter according to claim 102, wherein said TPL-2 kinase comprises TPL-2(30-467, D253N).

113. The composition of matter according to claim 102, wherein said TPL-2 kinase comprises TPL-2(30-467, D253N, S368A).

114. The composition of matter according to claim 102, wherein said p105 is selected from the group consisting of p105 (369-969), p105 (369-896), p105 (400-896), p105 (444-896), p105 (472-896), p105 (472-896, S894A), p105 (1-969, Δ803-893), p105 (1-969, Δ498-541), p105 (1-969, L842A), p105(498-806), p105(498-782), and p105(809-893).

115. The composition of matter according to claim 102, wherein said p105 comprises p105 (369-969).

116. The composition of matter according to claim 102, wherein said p105 comprises p105 (472-896).

117. The composition of matter according to claim 102, wherein said p105 comprises p105 (472-896, S894A).

118. The composition of matter according to claim 102, further comprising at least one nucleic acid sequence encoding a detectable tag, wherein said nucleic acid sequence encoding a detectable tag is operatively linked to at least one of said first nucleic acid and said second nucleic acid.

119. The composition of matter according to claim 118, wherein said detectable tag is selected from the group consisting of GFP, HA, myc, FLAG, 6HIS, maltose binding protein and GST.

120. The composition of matter according to claim 119, wherein said detectable tag is selected from the group consisting of FLAG and HA.

121. The composition of matter according to claim 120, wherein said detectable tag is FLAG, and wherein said nucleic acid sequence encoding FLAG is operatively linked to said first nucleic acid.

122. The composition of matter according to claim 120, wherein said detectable tag is HA, and wherein said nucleic acid sequence encoding HA is operatively linked to said second nucleic acid.

123. The composition of matter according to claim 120, wherein a nucleic acid sequence encoding detectable tag FLAG is operatively linked to said first nucleic acid; and a nucleic acid sequence encoding detectable tag HA is operatively linked to said second nucleic acid.

124. A composition of matter comprising an expression vector, said expression vector comprising a first nucleic acid comprising a first nucleic acid sequence encoding TPL-2(30-467) operatively linked to a FLAG tag, and a second nucleic acid comprising a second nucleic acid sequence encoding p105(369-896) operatively linked to an HA tag, wherein said first nucleic acid is under the control of baculovirus polyhedrin promoter and said second nucleic acid is under the control of p10 promoter.

125. A host cell transformed with a composition of matter according to any one of claims 102-124.

126. The host cell according to claim 125, wherein said host cell is an eukaryotic cell.

127. The host cell according to claim 126, wherein said eukaryotic cell is selected from the group consisting of a mammalian cell, fungal cell, avian cell, plant cell, and insect cell.

128. The host cell according to claim 127, wherein said host cell is SF9.

129. The host cell according to claim 127, wherein said host cell is HEK 293.

130. The host cell according to claim 125, wherein said host cell is a prokaryotic cell.

131. The host cell according to claim 130, wherein said host cell is E.Coli.

132. A method for producing TPL-2 kinase comprising the steps:
(a) providing a host cell according to any one of claims 125-131; and
(b) culturing said host cell under conditions sufficient to allow expression of TPL-2 kinase.

133. The method according to claim 132, further comprising the steps:
(c) lysing said host cell; and
(d) isolating TPL-2 kinase.

134. A method for producing p105 comprising the steps:
(a) providing a host cell according to any one of claims 125-131; and
(b) culturing said host cell under conditions sufficient to allow expression of p105.

135. The method according to claim 134, further comprising the steps:
(c) lysing said host cell; and
(d) isolating p105.

136. A method for producing TPL-2/p105 complex comprising the steps:
(a) providing a host cell according to any one of claims 125-131; and
(b) culturing said host cell under conditions sufficient to allow expression of TPL-2/p105 complex.

137. The method according to claim 136, further comprising the steps:
(c) lysing said host cells; and
(d) isolating TPL-2/p105 complex.

138. A method for isolating TPL-2 kinase comprising the steps:
(a) producing TPL-2/p105 complex according to claim 136 or 137;
(b) adding 8M urea sufficient to disrupt TPL-2/p105 complex; and
(c) isolating TPL-2 kinase.

139. A method for isolating p105 comprising the steps:
(a) producing TPL-2/p105 complex according to claim 136 or 137;
(b) adding 8M urea sufficient to disrupt TPL-2/p105 complex; and
(c) isolating p105.

140. TPL-2 kinase produced according to any one of claims 132, 133, or 138.

141. p105 produced according to any one of claims 134, 135, or 139.

142. TPL-2/p105 complex produced according to any one of claims 136 or 137.


SEQUENCE LISTING (9 sequences)

SEQ ID NO: 1 - DNA, 2720 bp, Rattus norvegicus
  CDS (318)..(1742)
ggaatttcccatcgcgggggctcgggtgttctgggccagccggcaggccctttctgtttacggagagaaaggggaaatggaaaaggcggggaggacgctggcgtcggctacgccgccccggggccagttcagacgccgagagtccggggctgcagcgtaccgctcctcccgctgcggatcgcccggcctttggtcggccgccggtcgtccggacgcccgtacgtctggctcccgctggcaagccacccgctgcccaccaagcccgagctccgggcgggcacacggaacactcagactccccagcaggcaccacagtgatggagtacatgagcaccggaagcgacgagaaagaagagattgatttattaattaaccatttaaacgtgtcggaagtcctggacatcatggagaacctttatgcaagtgaagagcctgcagtgtatgagcccagtctgatgaccatgtgtccagacagcaatcaaaacaaggaacattcagagtcgctgcttcggagtggccaggaggtgccctggttgtcgtctgtcagatatgggactgtggaggatctgcttgcatttgcaaaccatatctcgaatacgacaaagcatttttacagatgtcggccccaagaatctgggattttattaaatatggtaatcagtccccagaatggtcgctaccaaatcgactcggatgttctccttgtcccgtggaagctgacgtacaggagcattggttctggtttcgttcctcggggggcctttggaaaagtgtacttagcacaagacatgaagacaaagaaaagaatggcatgtaaactgatccctgtagatcagtttaagccatcagatgtggaaatccaggcctgcttccggcacgagaacattgccgagttatacggtgcggtcctatggggcgacactgtccatctcttcatggaagccggcgagggagggtctgtcctggagaagctggagagctgtgggcccatgagagaatttgaaattatctgggtgacaaagcacgttctcaagggacttgattttctgcactccaagaaagtcatccaccacgatatcaaacctagcaacattgtattcatgtctacgaaagctgtgttggtagattttggcctgagtgttcaaatgacagaagatgtctatctccccaaggacctccggggaacagagatctacatgagccctgaggtgattctgtgcaggggccattccacaaaagcagacatctacagccttggagccacgctcattcacatgcagacaggcaccccaccctgggtgaagcgctaccctcgatcggcctatccctcctacctgtacataatccacaagcaggcacctcccctggaagatattgctggtgactgcagtccaggcatgagggagctgatagaagccgccctggagaggaaccccaaccaccgcccaaaagcagcagacctactgaaacacgaagccctgaatcccccaagagaggaccagccacggtgtcagagtctggactctgccctctttgaccggaagaggctgctgagcaggaaggagctagaacttcctgagaacattgctgattcatcatgcacaggaagcaccgaggagtctgaagtgctcaggagacagcgttccctctacattgatctcggagctctggctggctacttcaatattgttcgtggtccaccaaccctggaatatggctgatggatgactctattggcaacagtagggcggatatttctctcctggatgttggtttcacagatcctacacagcagctctggatagtgaattttacccaatttttttaggaagcagggaggaggtctctagtgacacaagaatgtcaaagccctggccccctttgtgaagctcctctggcatgttccagagcccaaggttctcatttctcaggtggtgggactggacaaaagggagtggtgagctcaggaaagaatcatttctgatgacaattctattcactttgcactttaatggacattaaaaaatagctctcacaagatagtaacctaaaatacctgtttttggttcttatataaccatgggttcttcattcaactcagaagacctgatctgtgtatatatttgtgtgtattatatggtaactctttgtaccttggttggtagagtctagtataagtttagttaatagtattttgggtggatagaacaactctaatattacagcaattcactggactagtgtctcacaaatgactgatttactcagagccattaagcagcaggccactagtgagagtttctgttatgttcctatggaaacactgtgtattgtacgtgctatgcttaaaacatttaaaacacaatgttttaaatgtggacagaactgtgtaaaccacataatttctgtacatcccaaaggatgagaaatgtgaccttcaagaaaatggaaacatttgtaaattctttgtagtgatacctttgtaattaatgaaactatttttctttaaagtgtttctatattaaaaatagcatactgtgtatgttttattccaaaattccttcatgaatctttcatatatatatgtgtatatattttaacattgtaaagtatgagtattcttatttaaagtatatttttacattatgcaaatgaacttcaacgttttagtccaatgtgactggtcaaataaaccaaataaactgagtattttgtcttaa

SEQ ID NO: 2 - protein, 467 aa, Rattus norvegicus
MEYMSTGSDEKEEIDLLINHLNVSEVLDIMENLYASEEPAVYEPSLMTMCPDSNQNKEHSESLLRSGQEVPWLSSVRYGTVEDLLAFANHISNTTKHFYRCRPQESGILLNMVISPQNGRYQIDSDVLLVPWKLTYRSIGSGFVPRGAFGKVYLAQDMKTKKRMACKLIPVDQFKPSDVEIQACFRHENIAELYGAVLWGDTVHLFMEAGEGGSVLEKLESCGPMREFEIIWVTKHVLKGLDFLHSKKVIHHDIKPSNIVFMSTKAVLVDFGLSVQMTEDVYLPKDLRGTEIYMSPEVILCRGHSTKADIYSLGATLIHMQTGTPPWVKRYPRSAYPSYLYIIHKQAPPLEDIAGDCSPGMRELIEAALERNPNHRPKAADLLKHEALNPPREDQPRCQSLDSALFDRKRLLSRKELELPENIADSSCTGSTEESEVLRRQRSLYIDLGALAGYFNIVRGPPTLEYG

SEQ ID NO: 3 - DNA, 2763 bp, Homo sapiens
  CDS (367)..(1770)
ggatcccagtggcccggcgtgctcggctcccacaggcctgcagccagcatcgcaccgaaccttcggggggccgcggctggagcgctcggccggcgtgggagcgcaaggccgcagatgcaatcttcttaccgcgaagaagccaggggaataggtagccacatcttgtttgcagataagaaaggaagctaacgcagtatctgcaaagccaggagtctgactcagtacttttctcactcatgcatacaagcagctaaaaatgacacagcttatttaccatgcccctgacactgcactgagcactttatgagcttgaactctgttaatctcacgaccacctcatgagactctccagaaagagcaacagtaatggagtacatgagcactggaagtgacaataaagaagagattgatttattaattaaacatttaaatgtgtctgatgtaatagacattatggaaaatctttatgcaagtgaagagccagcagtttatgaacccagtctaatgaccatgtgtcaagacagtaatcaaaacgatgagcgttctaagtctctgctgcttagtggccaagaggtaccatggttgtcatcagtcagatatggaactgtggaggatttgcttgcttttgcaaaccatatatccaacactgcaaagcatttttatggacaacgaccacaggaatctggaattttattaaacatggtcatcactccccaaaatggacgttaccaaatagattccgatgttctcctgatcccctggaagctgacttacaggaatattggttctgattttattcctcggggcgcctttggaaaggtatacttggctcaagatataaagacgaagaaaagaatggcgtgtaaactgatcccagtagatcaatttaagccatctgatgtggaaattcaggcttgcttccggcacgagaacatcgcagagctgtatggcgcagtcctgtggggtgaaactgtccatctctttatggaagcaggcgagggagggtctgttctggagaaactggagagctgtggaccaatgagagaatttgaaattatttgggtgacaaagcatgttctcaagggacttgattttctacactcaaagaaagtgatccatcatgatattaaacctagcaacattgttttcatgtccacaaaagctgttttggtggattttggcctaagtgttcaaatgaccgaagatgtctattttcctaaggacctccgaggaacagagatttacatgagcccagaggtcatcctgtgcaggggccattcaaccaaagcagacatctacagcctgggggccacgctcatccacatgcagacgggcaccccaccctgggtgaagcgctaccctcgctcagcctatccctcctacctgtacataatccacaagcaagcacctccactggaagacattgcagatgactgcagtccagggatgagagagctgatagaagcttccctggagagaaaccccaatcaccgcccaagagccgcagacctactaaaacatgaggccctgaacccgcccagagaggatcagccacgctgtacgagtctggactctgccctcttggagcgcaagaggctgctgagtaggaaggagctggaacttcctgagaacattgctgattcttcgtgcacaggaagcaccgaggaatctgagatgctcaagaggcaacgctctctctacatcgacctcggcgctctggctggctacttcaatcttgttcggggaccaccaacgcttgaatatggctgaaggatgccatgtttgcctctaaattaagacagcattgatctcctggaggctggttctgctgcctctacacaggggcccgttacagtgaatggtgccattttcgaaggagcagtgtgacctcctgtgacccatgaatgtgcctccaagcggccctgtgtgtttgacatgtgaagctatttgatatgcaccaggtctcaaggttctcatttctcaggtgacgtgattctaaggcaggaatttgagagttcacagaaggatcgtgtctgctgactgtttcattcactgtgcactttgctcaaaattttaaaaataccaatcacaaggataatagagtagcctaaaattactattcttggttcttatttaagtatggaatattcattttactcagaatagcctgttttgtgtatattggtgtatattatataactctttgagcctttattggtaaattctggtatacattgaattcattataatttgggtgactagaacaacttgaagattgtagcaataagctggactagtgtcctaaaaatggctaactgatgaattagaagccatctgacagacggccactagtgacagtttcttttgtgttcctatggaaacattttatactgtacatgctatgctgaagacattcaaaacgtgatgttttgaatgtggataaaactgtgtaaaccacataattttgtacatccaaggatgaggtgtgacctttaagaaaaatgaaaacttttgtaaattattgatgattttgtaattcttatgactaaattttcttttaagcatttgtatattaaaatagcatactgtgtatgttttatatcaaatgccttcatgaatctttcatacatatatatatttgtaacatgtaaagtatgtgagtagtcttatgtaaagtatgtttttacattatgcaaataaaacccaatacttttgtccaatgtggttggtcaaatcaactgaataaattcagtattttgcctt

SEQ ID NO: 4 - protein, 467 aa, Homo sapiens [sequence omitted]

SEQ ID NO: 5 - protein, 969 aa, Homo sapiens
MAEDDPYLGRPEQMFHLDPSLTHTIFNPEVFQPQMALPTADGPYLQILEQPKQRGFRFRYVCEGPSHGGLPGASSEKNKKSYPQVKICNYVGPAKVIVQLVTNGKNIHLHAHSLVGKHCEDGICTVTAGPKDMVVGFANLGILHVTKKKVFETLEARMTEACIRGYNPGLLVHPDLAYLQAEGGGDRQLGDREKELIRQAALQQTKEMDLSVVRLMFTAFLPDSTGSFTRRLEPVVSDAIYDSKAPNASNLKIVRMDRTAGCVTGGEEIYLLCDKVQKDDIQIRFYEEEENGGVWEGFGDFSPTDVHRQFAIVFKTPKYKDINITKPASVFVQLRRKSDLETSEPKPFLYYPEIKDKEEVQRKRQKLMPNFSDSFGGGSGAGAGGGGMFGSGGGGGGTGSTGPGYSFPHYGFPTYGGITFHPGTTKSNAGMKHGTMDTESKKDPEGCDKSDDKNTVNLFGKVIETTEQDQEPSEATVGNGEVTLTYATGTKEESAGVQDNLFLEKAMQLAKRHANALFDYAVTGDVKMLLAVQRHLTAVQDENGDSVLHLAIIHLHSQLVRDLLEVTSGLISDDIINMRNDLYQTPLHLAVITKQEDVVEDLLRAGADLSLLDRLGNSVLHLAAKEGHDKVLSILLKHKKAALLLDHPNGDGLNAIHLAMMSNSLPCLLLLVAAGADVNAQEQKSGRTALHLAVEHDNISLAGCLLLEGDAHVDSTTYDGTTPLHIAAGRGSTRLAALLKAAGADPLVENFEPLYDLDDSWENAGEDEGVVPGTTPLDMATSWQVFDILNGKPYEPEFTSDDLLAQGDMKQLAEDVKLQLYKLLEIPDPDKNWATLAQKLGLGILNNAFRLSPAPSKTLMDNYEVSGGTVRELVEALRQMGYTEAIEVIQAASSPVKTTSQAHSLPLSPASTRQQIDELRDSDSVCDSGVETSFRKLSFTESLTSGASLLTLNKMPHDYGQEGPLEGKI

SEQ ID NO: 6 - DNA, 18 bp, Artificial
  primer_bind: PCR primer element
actgggctcatacactgc

SEQ ID NO: 7 - DNA, 22 bp, Artificial
  primer_bind: PCR primer element
ggcggcttggtgactctagata

SEQ ID NO: 8 - DNA, 21 bp, Artificial
  primer_bind: PCR primer element
gctcgggcctgctttgaacac

SEQ ID NO: 9 - DNA, 21 bp, Artificial
  primer_bind: PCR primer element
gctcgggcctgctttgaacac